(19) United States
(12) Patent Application Publication
(10) Pub. No.: US 2025/0333487 A1
(43) Pub. Date: Oct. 30, 2025

(54) ACTIVATABLE DUAL-ANCHORED MASKED MOLECULES AND METHODS OF USE THEREOF

(71) Applicant: CytomX Therapeutics, Inc., South San Francisco, CA (US)

(72) Inventors: Ellaine Anne Mariano FOX, San Carlos, CA (US); Madan M. PAIDHUNGAT, San Francisco, CA (US); W. Michael KAVANAUGH, Orinda, CA (US); Vangipuram S. RANGAN, Pleasant Hil, CA (US); Andrew JANG, San Jose, CA (US); ANNA Faith NGUYEN, San Francisco, CA (US)

(21) Appl. No.: 18/849,503

(22) PCT Filed: Mar. 24, 2023
(86) PCT No.: PCT/US2023/064937

§ 371 Date: Sep. 22, 2024

Related U.S. Application Data
(60) Provisional application No. 63/323,718, filed on Mar. 25, 2022.

(51) Int. Cl. C07K 16/18; C07K 16/28

(52) U.S. Cl. CPC C07K 16/18; C07K 16/2827; C07K 2317/569; C07K 2319/50

(57) ABSTRACT

Provided herein are activatable target-binding proteins comprising a target-binding protein (TB) that specifically binds to a target; a masking moiety (MM) coupled to the TB, wherein the MM and the TB are tethered by a non-alpha-carbon covalent bond and the MM inhibits the binding of the TB to the target when the activatable target-binding protein; and a cleavable moiety (CM) coupled to the TB, wherein the CM is a polypeptide that functions as a substrate for a protease and positioned between the TB and the MM.

### CROSS-REFERENCE TO RELATED APPLICATION

This application is a 35 U.S.C. 371 National Phase Entry Application of PCT/US2023/064937, filed Mar. 24, 2023, which claims priority benefit of U.S. Provisional Application No. 63/323,718, filed Mar. 25, 2022, which is incorporated herein by reference in its entirety.

### SEQUENCE LISTING

The Sequence Listing filed with this application by EFS, which is entitled “4862-121PCT.xml,” was created on Mar. 22, 2023 and is 500,152 bytes in size, is hereby incorporated by reference in its entirety.

### TECHNICAL FIELD

The present disclosure relates to the field of biotechnology, and more specifically, to activatable molecules.

### BACKGROUND

Antibody-based therapies have provided proven effective treatments for various diseases. However, in some cases, toxicities due to broad target expression have limited their therapeutic effectiveness. In addition, antibody-based therapeutics have exhibited other limitations such as rapid clearance from the circulation following administration.

Activatable antibodies comprising mask peptides binding to the antibodies are therapeutic agents with lower toxicities compared to regular antibodies. The masks can inhibit the antibodies' activity by interrupting the binding of the antibodies with their target molecules. In an activating environment (e.g., when the activatable antibodies are delivered to a tumor), the masks are removed so the antibodies can bind to their target molecules to resume their functions. However, toxicities may arise if a portion of the activatable antibodies are in an unmasked state allowing for target binding outside the activating environment.

Accordingly, there is an ongoing need for activatable molecules that minimize target binding outside the activating environment.

### SUMMARY OF THE INVENTION

The present disclosure provides dual-anchored activatable target-binding proteins and related compositions and methods.

In one aspect, the present disclosure provides a dual-anchored activatable target-binding protein comprising: a target-binding protein (TB) that specifically binds to a target; a masking moiety (MM) coupled to the TB, wherein the MM inhibits binding of the TB to the target; and a cleavable moiety (CM) coupled to the TB and positioned between the TB and the MM, wherein the CM is a polypeptide that functions as a substrate for a protease, and further comprising a non-alpha-carbon covalent bond tethering the MM and the TB.

In some embodiments, the TB is an antigen-binding protein (AB). In some embodiments, the activatable target-binding protein has a lower target-binding activity compared to a single-anchored activatable target-binding protein lacking the non-alpha-carbon covalent bond. In some embodiments, the non-alpha-carbon covalent bond is an isopeptide bond. In some embodiments, the isopeptide bond is between a lysine and a glutamate or aspartate residue. In some embodiments, the non-alpha-carbon covalent bond is between functional groups substituted into an alpha-carbon in the MM and the AB. In some embodiments, the isopeptide bond is between the gamma-carboxyamide group of glutamine and epsilon-amino group of lysine sidechains. In some embodiments, the non-alpha-carbon covalent bond is an ester bond between threonine and glutamine. In some embodiments, the non-alpha-carbon covalent bond is a thioester bond between cysteine and glutamine. In some embodiments, the non-alpha-carbon covalent bond is a thioether bond between cysteine and tyrosine. In some embodiments, the non-alpha-carbon covalent bond is formed by crosslinking between histidine and tyrosine (e.g., this type of histidine-tyrosine crosslinking is known to exist in cytochrome e oxidase enzymes). In some embodiments, the non-alpha-carbon covalent bond is a nitrogen-oxygen-sulfur (NOS) bond formed between lysine and cysteine. In some embodiments, the non-alpha-carbon covalent bond is a disulfide bond. In some embodiments, the disulfide bond is formed between a first cysteine and a second cysteine, wherein the first cysteine is within the MM and the second cysteine is within the TB, the first cysteine is within a peptide coupled to the MM and the second cysteine is within the TB, or the first cysteine is within the MM and the second cysteine is within a peptide coupled to the TB.

In some embodiments, the activatable target-binding protein further comprises a second CM, wherein the second CM is positioned between the MM and the non-alpha-carbon covalent bond, the second CM is within the MM and up to 5 amino acids away from a cysteine forming the non-alpha-carbon covalent bond, or the second CM is within the TB and at up to 5 amino acids away from a cysteine forming the non-alpha-carbon covalent bond. In some embodiments, the first and the second CMs are substrates of different proteases. In some embodiments, the first and the second CMs are substrates of the same protease.

In some embodiments, the protease is produced by a tumor in a subject. In some embodiments, the AB is an antibody, a Fab fragment, a F(ab′)₂ fragment, an scFv, an scAb, a dAb, or a single domain antibody. In some embodiments, the AB is a single domain antibody. In some embodiments, the AB is an Fc-tagged single domain antibody. In some embodiments, the AB is a bispecific antibody. In some embodiments, the bispecific antibody is a bispecific T Cell engager (BiTE) or a dual-affinity retargeting antibody (DART). In some embodiments, the AB is a multispecific antibody. In some embodiments, the non-alpha-carbon covalent bond is between the MM and the single domain antibody. The present disclosure includes a dual-anchored activatable macromolecule comprising a bispecific or multispecific AB, wherein each AB in the bispecific or multispecific AB has a dual anchored MM. The present disclosure also includes a dual-anchored activatable macromolecule comprising a bispecific or multispecific AB, wherein at least one AB in the bispecific or multispecific AB has a dual anchored MM and at least one AB in the bispecific or multispecific AB has a single anchored MM. The present disclosure also includes a dual-anchored activatable macromolecule comprising a bispecific or multispecific AB, wherein at least one AB in the bispecific or multispecific AB has a dual anchored MM and at least one AB in the bispecific or multispecific AB does not have a MM.

In some embodiments, the non-alpha-carbon covalent bond is between the MM and a fragment crystallizable region (Fc) region or domain coupled to the TB. In some embodiments, the MM may comprise an epitope of the TB. In some embodiments, the MM does not comprise a subsequence of four or more consecutive amino acid residues of a native TB. In some embodiments, the MM does not comprise a subsequence of four or more consecutive amino acid residues of the target bound by the TB. In some embodiments, the MM may comprise a subsequence of less than four consecutive amino acid residues of a native TB. In some embodiments, the MM does not comprise an epitope of the TB. In some embodiments, the MM has a dissociation constant for binding to the TB that is greater than a dissociation constant of the TB for binding to the target. In some embodiments, the MM is a polypeptide of from 2 to 40 amino acids in length.

In some embodiments, the activatable target-binding protein comprises a linker between the MM and the CM. In some embodiments, the activatable target-binding protein comprises a linker between CM and the TB. In some embodiments, the activatable target-binding protein comprises a first linker between the MM and the CM and a second linker between the CM and the TB.

In another aspect, the present disclosure provides a composition comprising the activatable target-binding protein herein. In some embodiments, the composition is a pharmaceutical composition.

In another aspect, the present disclosure provides a container, vial, syringe, injector pen, or kit comprising at least one dose of the composition herein.

In another aspect, the present disclosure provides a nucleic acid comprising a sequence encoding the activatable target-binding protein herein.

In another aspect, the present disclosure provides a vector comprising the nucleic acid herein.

In another aspect, the present disclosure provides a cell comprising the nucleic acid or the vector herein.

In another aspect, the present disclosure provides a conjugated activatable target-binding protein comprising the activatable target-binding protein herein conjugated to an agent. In some embodiments, the agent is a therapeutic agent, a targeting moiety, or a detectable moiety.

In another aspect, the present disclosure provides a method of treating a subject in need thereof comprising administering to the subject a therapeutically effective amount of the activatable target-binding protein, the composition, or the conjugated activatable target-binding protein herein. In some embodiments, the subject has been identified or diagnosed as having a cancer.

In another aspect, the present disclosure provides a method of producing an activatable target-binding protein, comprising: culturing the cell in a culture medium under a condition sufficient to produce the activatable target-binding protein; and recovering the activatable target-binding protein from the cell or the culture medium.

In some embodiments, the method further comprises isolating the activatable target-binding protein recovered from the cell or the culture medium. In some embodiments, isolating the activatable target-binding protein is performed using a protein purification tag and/or size exclusion chromatography. In some embodiments, the method further comprises formulating the activatable target-binding protein into a pharmaceutical composition.

In another aspect, the present disclosure provides a method of producing a dual-anchored activatable protein comprising: engineering a cysteine residue at a disulfide bonding site in a masking moiety (MM) of the dual-anchored activatable protein; engineering a cysteine residue at a disulfide bonding site in a target-binding protein (TB) of the dual-anchored activatable protein, wherein the MM and the TB are coupled and a cleavable moiety (CM) is positioned between the MM and the TB; expressing the dual-anchored activatable protein; and recovering the dual-anchored activatable protein, wherein the MM and the TB are tethered at their disulfide bonding sites in the recovered dual-anchored activatable protein. In this disclosure the phrases dual-anchored activatable protein and dual-anchored activatable macromolecule are used interchangeably.

In another aspect, the present disclosure provides a method of producing a dual-anchored activatable macromolecule comprising: engineering an arginine or lysine residue at an isopeptide bonding site in a masking moiety (MM) of the dual-anchored activatable macromolecule and/or engineering an aspartate or glutamate residue at an isopeptide bonding site in a target-binding protein (TB) of the dual-anchored activatable macromolecule, wherein the MM and the TB are coupled and a cleavable moiety (CM) is positioned between the MM and the TB; expressing the dual-anchored activatable macromolecule; and recovering the dual-anchored activatable macromolecule, wherein the MM and the TB are tethered at their isopeptide bonding sites in the recovered dual-anchored activatable macromolecule. In another aspect, the present disclosure provides a method of producing a dual-anchored activatable macromolecule comprising: engineering the isopeptide bonding sites such that a gamma-carboxyamide group of glutamine is available and configured to form an isopeptide bond with an epsilon-amino group of a lysine sidechain.

In another aspect, the present disclosure provides a method of producing a dual-anchored activatable macromolecule comprising: engineering an aspartate or glutamate residue at an isopeptide bonding site in a masking moiety (MM) of the dual-anchored activatable macromolecule and/or engineering an arginine or lysine residue at an isopeptide bonding site in a target-binding protein (TB) of the dual-anchored activatable macromolecule, wherein the MM and the TB are coupled and a cleavable moiety (CM) is positioned between the MM and the TB; expressing the dual-anchored activatable macromolecule; and recovering the dual-anchored activatable macromolecule, wherein the MM and the TB are tethered at their isopeptide bonding sites in the recovered dual-anchored activatable macromolecule.

In another aspect, the present disclosure provides a method of making a dual-anchored activatable macromolecule comprising providing a MM comprising a non-alpha-carbon covalent bond-forming amino acid configured to form a non-alpha-carbon covalent bond with a non-alpha-carbon covalent bond-forming amino acid in a TB that is coupled to the MM.

In another aspect, the present disclosure provides a method of making a dual-anchored activatable macromolecule comprising providing a MM comprising a cysteine configured to form a non-alpha-carbon covalent bond with a non-alpha-carbon covalent bond-forming amino acid in a TB that is coupled to the MM.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A schematically shows the dynamic equilibrium between a fully mask-bound conformational state (left), an intermediate conformational state in which one mask is dynamically dissociated from the binding site of the target-binding protein (or antibody) (middle), and a conformational state in which two masks are dynamically dissociated from the binding site of the target-binding protein (or antibody) (right).

FIG. 1B shows the arrangements of components in example activatable antibodies. The lines connecting the MM and the AB indicate non-alpha-carbon covalent bonds. While an AB is exemplified, the present disclosure includes use of any desired protein and is not limited to antibodies and can include any target-binding protein (TB).

FIGS. 2A-2D show schematics of example activatable molecules.

FIGS. 3A-3C show three types of example activatable molecules.

FIG. 4A is a schematic of an illustrative dual-anchored BC2T-Nb with the N-terminus of the MM covalently linked to the Nb with engineered non-alpha-carbon covalent bonds, e.g., disulfide bonds and the C-terminus of the MM covalently linked to the Nb with a CM. FIG. 4B is a schematic of single-anchored BC2T-Nb. FIG. 4C depicts the cleavage reaction of the dual-anchored BC2T-Nb resulting in the MM being single-anchored by the engineered non-alpha-carbon covalent bond, e.g., disulfide bond. The BC2T-Nb molecule is exemplified as a non-limiting proof-of-concept example and a person skilled in the art will understand that the structure depicted and described in this disclosure extends to use of any type of masking moiety and any desired protein without undue experimentation.

FIG. 5A is a schematic of an illustrative dimeric Fc-tagged version of a dual-anchored BC2T-Nb with the N-terminus of the MM covalently linked to the Nb with engineered non-alpha-carbon covalent bond, e.g., disulfide bonds and the C-terminus of the MM covalently linked to the Nb with a CM. FIG. 5B is a schematic of a dimeric Fc-tagged control molecule (i.e., the MM and Nb are not tethered by a non-alpha-carbon covalent bond, e.g., disulfide bond) to show the difference in binding between the dual-anchored BC2T-Nb and the single-anchored BC2T-Nb. FIG. 5C depicts that the cleavage reaction of the Fc-tagged dual-anchored BC2T-Nb results in the MM being single-anchored by the engineered non-alpha-carbon covalent bond, e.g., disulfide bond. The BC2T-Nb molecule is exemplified as a non-limiting proof-of-concept example and a person skilled in the art will understand that the structure depicted and described in this disclosure extends to use of any type of masking moiety and any desired protein without undue experimentation.

FIG. 6 is an image of an SDS-PAGE gel run under non-reducing conditions. The gel was loaded as follows. (1) single-domain antibody with no MM attached (ProC649; SEQ ID NO: 1); (2) product of ProC649 and MMP14 (ProC649+MMP14); (3) product of ProC649 and MMP9 (ProC649+MMP9); (4) single-domain antibody with MM attached with a CM 1490DNI (ProC653; SEQ ID NO: 2); (5) product of ProC653 and MMP14 (ProC653+MMP14); (6) single-domain antibody with MM attached with a CM PLGLAG (SEQ ID NO: 17) (ProC654; SEQ ID NO: 3); (7) product of ProC654 and MMP9 (ProC654+MMP9); (8) MMP14; and (9) MMP9.

FIG. 7 shows sensorgram trace binding of intact and activated ProC653 and ProC654 along with the ProC649 control. ProC649 which does not have an MM attached bound the biotinylated BC2 tag peptide on the biosensor tip. Intact ProC653 and ProC654 show no binding to the biotinylated BC2 tag peptide, but binding was recovered upon activation with either MMP14 or MMP9, respectively.

FIG. 8 is an image of an SDS-PAGE gel run under non-reducing (top) and reducing conditions (bottom). The gel was loaded as follows: (1) single-domain antibody with no MM attached (ProC649); (2) single-domain antibody with MM attached with a CM 1490DNI (ProC653); (3) product of ProC653 and uPA (ProC653+uPA); (4) single-domain antibody with MM dually anchored with engineered cysteines Q3C and Q157C and a CM 1490DNI (ProC994; SEQ ID NO: 4); (5) product of ProC994 and uPA (ProC994+uPA); (6) single-domain antibody with MM dually anchored with engineered cysteines Q3C and W155C and a CM 1490DNI (ProC995; SEQ ID NO: 5); (7) product of ProC995 and uPA (ProC995+uPA); (8) single-domain antibody with MM dually anchored with engineered cysteines D3C and F154C and a CM 1490DNI (ProC996; SEQ ID NO: 6); (9) product of ProC996 and uPA (ProC996+uPA).

FIG. 9 is an image of an SDS-PAGE gel run under non-reducing (top) and reducing conditions (bottom). The gel was loaded as follows: (1) Fc-tagged single-domain antibody with no MM attached (ProC1283; SEQ ID NO: 7); (2) product of ProC1283 and uPA (ProC1283+uPA), (3) Fc-tagged single-domain antibody with MM attached with a CM 1490DNI (ProC1284; SEQ ID NO: 8); (4) product of ProC1284 and uPA (ProC1284+uPA); (5) Fc-tagged single-domain antibody with MM dually anchored with engineered cysteines Q3C and Q157C and a CM 1490DNI (ProC1285; SEQ ID NO: 9); (6) product of ProC1285 and uPA (ProC1285+uPA): (7) Fc-tagged single-domain antibody with MM dually anchored with engineered cysteines D3C and F154C and a CM 1490DNI (ProC1287; SEQ ID NO: 11); (8) product of ProC1287 and uPA (ProC1287+uPA); (9) uPA.

FIG. 10 provides the results of an ELISA binding assay to determine the shift in ability of the molecules to bind free masking peptide bound to the plate: Fc-tagged single-domain antibody with no MM attached (ProC1283), product of ProC1283 and uPA (ProC1283+uPA), Fc-tagged single-domain antibody with MM attached with a CM 1490DNI (ProC1284), product of ProC1284 and uPA (ProC1284+uPA), Fc-tagged single-domain antibody with MM dually anchored with engineered cysteines Q3C and Q157C and a CM 1490DNI (ProC1285); product of ProC1285 and uPA (ProC1285+uPA), Fc-tagged single-domain antibody with MM dually anchored with engineered cysteines D3C and F154C and a CM 1490DNI (ProC1287), and product of ProC1287 and uPA (ProC1287+uPA). The results show that the single domain antibodies with the dual-anchored masks were unable to bind the peptide on the plate. However, treatment of the dual-anchored molecules with protease restored the binding of the molecules similar to the single-anchored molecule (ProC1283).

FIGS. 11A-11I depict exemplary dual-anchored multispecific activatable antibodies. Oval shapes indicate components of ABs, which may be heavy chain variable regions (V_{H}), light chain variable regions (V_{L}), heavy chain constant regions (C_{H}), light chain constant regions (C_{L}), single variable domain on a heavy chain (VHH), or single chain variable fragments (scFvs). Triangles indicate MMs. The MMs are coupled with the AB components via CMs (with optional linkers) and tethered with the ABs by disulfide bonds (or non-alpha-carbon covalent bonds) (with optional linker(s) and/or CM(s) between the MM and the residue that forms the non-alpha-carbon covalent bond with the AB).

FIGS. 12A-12B depict a three-dimensional structure of an activatable target binding protein (activatable anti-PDL 1 antibody; SEQ ID NOS: 562-563) obtained using BIOVIA Discovery Studios from Dessault Systemes software. FIG. 12A shows the magnified region of the structure with solvent accessible residues within 2-5 angstroms of residues in the header region or N-terminus of the mask moiety, with a second view of the structure rotated 90 degrees. Residues identified by mutagenesis to interact with the mask are indicated with an asterisk. FIG. 12B shows a three-dimensional structure of the Fab and prodomain (including the labeled mask moiety). The Fab domain is rendered in space-filling form with the prodomain rendered in the Ca backbone form.

FIGS. 13A-13D depict homology-based three-dimensional models of antibody structures corresponding to J43v2/anti-mouse PD1 Fab (FIG. 13A; SEQ ID NOs: 568-569), anti-CD166 (FIG. 13B; SEQ ID NOs: 572-573), and anti-PD1 (FIGS. 13C-13D; SEQ ID NOs: 570-571). FIGS. 13A-13C were modeled using BIOVIA Discovery Studio and FIG. 13D was modeled using AlphaFold2 and rendered in BIOVIA Discovery Studio. CDRs are shown in dark grey in each figure.

The figures herein are for illustrative purposes only and are not necessarily drawn to scale.

### DETAILED DESCRIPTION

### Overview

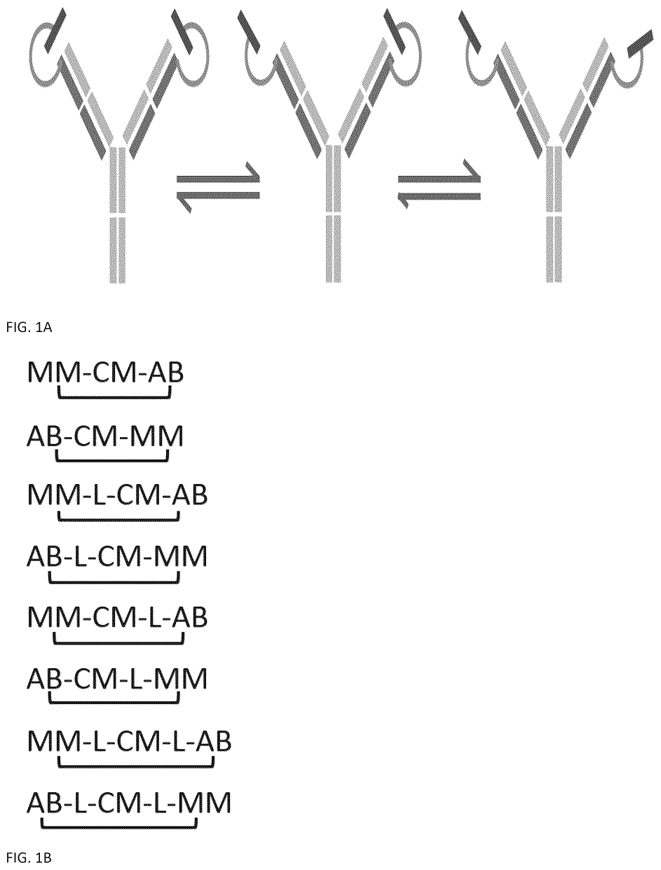

Provided herein are activatable molecules comprising at least one mask anchored at two points either directly or indirectly to at least one polypeptide of an otherwise active binding moiety (“activatable dual-anchored masked target-binding protein” or “activatable target-binding protein”). In some aspects as detailed and depicted in this disclosure, the activatable target-binding protein comprises a complex of more than one polypeptide. In some aspects as detailed and depicted in this disclosure, the mask is covalently bonded to one polypeptide of the activatable target-binding protein in two anchoring positions. In some aspects as detailed and depicted in this disclosure, the mask is covalently bonded to two polypeptides of the activatable target-binding protein, e.g., anchored at one anchoring position to a first polypeptide and anchored at a second anchoring position to a second polypeptide. In solution, masked activatable target-binding protein molecules lacking the dual-anchored structure described herein exist in a dynamic equilibrium state between conformational states in which one or more of the masks are actively bound to the binding site of a target-binding protein and conformational states in which one or more of the masks are not actively bound to the binding site of a target-binding protein as shown in FIG. 1A. This dynamic equilibrium may be referred to herein as “breathing” of a masked activatable target-binding protein molecule where the binding surface or binding surfaces of one or more target-binding proteins in the masked activatable target-binding protein molecule become available for binding to targets or other epitopes (including target binding outside the activating environment). Breathing occurs when the antigen binding site is exposed for a short period of time in some fraction of intact molecules due to equilibrium binding of the tethered mask, as depicted in FIG. 1A. The dual-anchored mask structure described herein reduces or inhibits dynamic dissociation between the mask and the target-binding protein, i.e., “breathing” of the activatable target-binding protein molecule. The dual-anchored mask appears to mask the otherwise target-binding protein with enhanced masking efficiency and/or reduced molecule populations in which one or more masks are dynamically dissociated from their corresponding target-binding proteins.

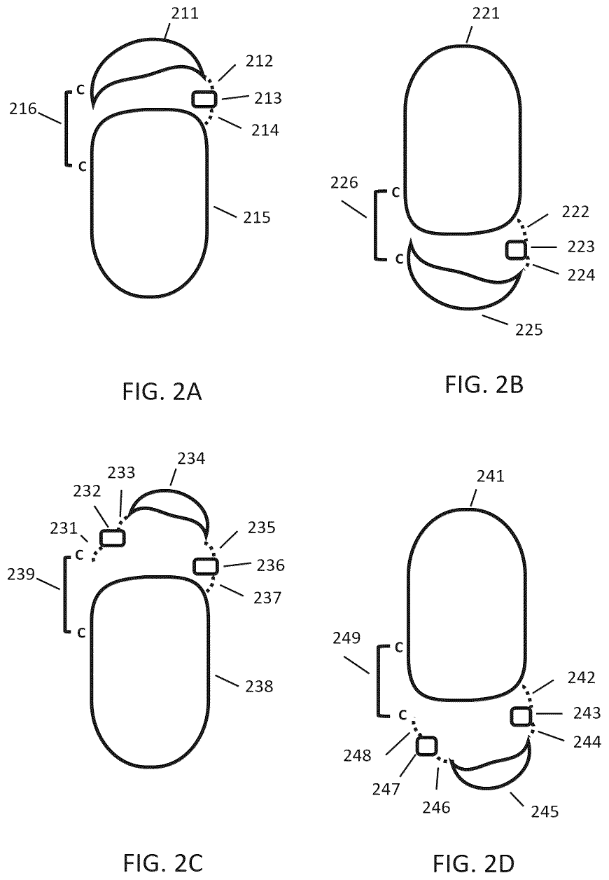

In one aspect, the activatable molecules may be activatable therapeutic macromolecules (“activatable target-binding protein”). In some aspects, the activatable therapeutic macromolecules may be activatable antibodies or any other desired protein, e.g., a therapeutic protein. The activatable molecules may comprise a target-binding protein (TB), a masking moiety (MM), and a cleavable moiety (CM) positioned between the MM and TB. In some aspects, the activatable molecules may comprise more than one CM, e.g., as shown in FIGS. 2C and 2D. For example, the activatable molecules may comprise a first cleavable moiety (CM1) and a second cleavable moiety (CM2). In some aspects, the activatable molecules may have a structure including CM1 between the MM and TB and a CM2 between the MM and the residue that forms the non-alpha-carbon covalent bond with the activatable molecule. Thus, in some aspects, the present disclosure includes a TB-CM1-MM-CM2 construct, where cleavage of CM1 and CM2 fully cleaves the MM from the activatable molecule at both anchorage sites. In some aspects, cleavage of both CM1 and CM2 results in full activation of the activatable target-binding protein. In some aspects, cleavage of both CM1 and CM2 is necessary for full activation of the activatable target-binding protein. In some aspects, cleavage of one of CM1 and CM2 is sufficient for activation of the activatable target-binding protein.

In some aspects, the activatable antibodies used in the context of the activatable dual-anchored masked antibodies of the present disclosure may comprise an antigen-binding protein (AB), a masking moiety (MM), and one or more cleavable moieties (CMs) positioned between the MM and AB. In general, the activatable molecules herein may be dual-anchored, i.e., the MM and the TB (e.g., AB) are coupled via a CM (or a CM1 and a CM2) and also tethered by one or more non-alpha-carbon covalent bonds. Such activatable molecules may have a lower target-binding activity compared to a counterpart activatable molecule without the non-alpha-carbon covalent bond (i.e., “single-anchored activatable molecules” or a “counterpart activatable target-binding protein”). Compared to single-anchored activatable molecules without the non-alpha-carbon covalent bond (e.g., disulfide bond) tethering their MM and TB, the enhanced masking efficiency of the MM in the dual-anchored activatable molecules describe herein may result in improved safety profiles, e.g., reduced toxicity and reduced target binding outside the activating environment.

Also provided herein are related compositions, kits, nucleic acids, vectors, and recombinant cells, as well as related methods, including methods of using and methods of producing any of the activatable molecules (e.g., activatable macromolecules, e.g., antibodies and other proteins) described herein.

### Definitions

Unless otherwise defined, all technical and scientific terms used herein have the same meaning as commonly understood by one of ordinary skill in the art to which this invention belongs. Methods and materials are described herein for use in the present disclosure; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art. For example ±20%, ±10%, or ±5%, may be within the intended meaning of the recited value, where appropriate.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are computed on the basis of weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition terms not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Furthermore, certain molecules, constructs, compositions, elements, moieties, excipients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of constructs, molecules, method steps, kits, or compositions described with respect to a construct, composition, or method is intended to and does find direct support for embodiments related to constructs, compositions, formulations, and methods described in any other part of this disclosure, even if those method steps, active agents, kits, or compositions are not re-listed in the context or section of that embodiment or aspect.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

### Activatable Target-binding Proteins

In one aspect, the present disclosure provides activatable target-binding proteins (TBs), for example, activatable dual-anchored masked antibodies (“activatable antibodies”) or another protein that specifically binds to a target. In some embodiments, the activatable antibody comprises a TB or an antigen-binding protein (AB) that specifically binds to a target; a cleavable moiety (CM) directly or indirectly covalently linked (also referred to as “coupled” or “fused”) to the TB (e.g., AB), wherein the CM is a polypeptide that functions as a substrate for a protease and positioned between the TB and a masking moiety (MM), wherein the MM and the TB are tethered by a non-alpha-carbon covalent bond and the MM inhibits the binding of the TB to the target. The term “fused” and grammatical variants thereof as used herein refer to a covalent linkage of the alpha-carbon backbone of the same polypeptide, e.g., by recombinant fusion. The terms “tether” and grammatical variants thereof as used herein refers to the linkage of two moieties (e.g., a MM and an AB in an activatable antibody) by a non-alpha-carbon covalent bond (e.g., a disulfide bond, and amide/isopeptide bond, between functional groups substituted into an alpha-carbon in the MM and the AB, or other bond that does not involve an alpha-carbon backbone bond). As used herein, the term “anchor” and grammatical variants thereof may include a moiety that is directly or indirectly covalently linked to another moiety (e.g., a TB and a CM or a MM and a CM). In some aspects, an isopeptide bond may be between a lysine residue and an aspartate or glutamate residue, or between a gamma-carboxyamide group of glutamine and epsilon-amino group of a lysine sidechain.

In some embodiments, activatable antibodies provide for reduced toxicity and/or side effects that could otherwise result from binding of the TB (e.g., AB) at non-treatment sites if the TB were not masked or otherwise inhibited from binding to the target. In the activatable TB, the MM may interfere with the binding of the TB to its target molecule. In single-anchored activatable antibodies (e.g., single-anchored activatable antibodies in which the MM and AB is not tethered by the non-alpha-carbon covalent bond), the MM's masking effect on the target-binding surface of the AB may be dynamic. Thus, in a given formulation comprising single-anchored activatable TB molecules, a portion of the activatable TB molecules in the formulation may be unmasked, albeit for a short period of time, due to breathing. At a sufficiently high concentration of the single-anchored activatable TBs, binding of such unmasked activatable TB with the target molecule may be significant and cause undesired toxicities or effects caused by target binding outside the activating environment.

As used herein, the term “activatable antibodies” or “activatable target-binding proteins” refer to an activatable antibody or an activatable target-binding protein, respectively, in its inactive (uncleaved or native) form. It will be apparent to the ordinarily skilled artisan that following modification of the CM of the activatable antibody or the activatable target-binding protein by at least one protease may result in a cleaved protein in which the MM is not interrupting the binding between the TB or the AB and its target. In some embodiments, cleavage of the CM by protease may result in release of the MM. The term “uncleaved” or “inactive” refers to the activatable TBs in the absence of cleavage of the CM by a protease, i.e., the activatable TBs in native form. As used throughout this disclosure, descriptions relating to activatable antibodies should be construed to also be applicable to activatable target-binding proteins. Thus, in one aspect, the dual-anchored activatable target-binding protein of the present disclosure may include an MM that inhibits binding of the AB to the target when the activatable target-binding protein is in an inactive state.

As used herein, following modification of the CM by at least one protease, the activatable TBs have been “cleaved” or “activated.” The term “activatable” when used in connection with the term “protein,” refers to a protein that exhibits diminished binding to a target relative to the corresponding “activated” protein that is generated by exposing the activatable protein to a cleaving agent (e.g., a protease).

As used herein, the terms “masking moiety” and “MM” are used interchangeably to refer to a peptide or protein that, when positioned proximal to a TB (e.g., an AB), interferes with binding of the TB to its target.

The terms “cleavable moiety” and “CM” are used interchangeably herein to refer to a peptide, the amino acid sequence of which comprises a substrate for a sequence-specific protease. In an activatable protein, the CM is positioned relative to the MM and TB, such that cleavage results in a molecule that is capable of binding to the biological target of the TB. Thus, the activatable protein exhibits a reduction in binding to the biological target as compared to the activated protein.

In some embodiments, an activatable TB may be designed by selecting a TB of interest and constructing the remainder of the activatable TB so that, when conformationally constrained, the MM provides for masking of the TB or reduction of binding of the TB to its target. Structural design criteria can be taken into account to provide for this functional feature.

Activatable antibodies may be provided in a variety of structural configurations. Exemplary formulae for activatable antibodies are provided below. It is contemplated that the N- to C-terminal order of the AB, MM and CM may be reversed within an activatable antibody. It is also contemplated that the CM and MM may overlap in amino acid sequence, e.g., such that the CM sequence recognized by the sequence specific-protease is at least partially contained within the MM. For example, activatable antibodies can be represented by the formula (in order from an amino (N) terminal region to carboxyl (C) terminal region) in FIG. 1B. The activatable antibodies may further comprise one or more linkers (Ls) between the MM and CM and/or between the CM and AB. The lines connecting the MM and the AB indicate non-alpha-carbon covalent bonds.

Exemplary configurations of the activatable antibodies are shown in FIGS. 2A-2D. FIG. 2A shows an example activatable antibody 210 comprising, in order from an amino (N) terminal region to carboxyl (C) terminal region, an MM 211, an optional linker 212, a CM 213, an optional linker 214 with the same or different sequences than 212, and an AB 215. The MM 211 and AB 215 are tethered by a non-alpha-carbon covalent bond 216. FIG. 2B shows an example activatable antibody 220 comprising, in order from an amino (N) terminal region to carboxyl (C) terminal region, an AB 221, an optional linker 222, a CM 223, an optional linker 224 with the same or different sequences than 222, and an MM 225. The AB 221 and MM 225 are tethered by a non-alpha-carbon covalent bond 226. FIG. 2C shows an example activatable antibody 230 comprising, in order from an amino (N) terminal region to carboxyl (C) terminal region, an optional linker 231, a CM 232, an optional linker 233, an MM 234, an optional linker 235, a CM 236, an optional linker 237, and an AB 238. The MM 234 and AB 238 are tethered by a non-alpha-carbon covalent bond 239. FIG. 2D shows an example activatable antibody 241 comprising, in order from an amino (N) terminal region to carboxyl (C) terminal region, an AB 241, an optional linker 242, a CM 243, an optional linker 244, an MM 245, an optional linker 246, a CM 247, and an optional linker 248. The AB 241 and MM 255 are tethered by a non-alpha-carbon covalent bond 249.

In some examples, the AB (e.g., the ABs in FIGS. 2A-2D) may comprise only one polypeptide. In such cases, the MM may be coupled to the polypeptide via the CM and tethered to the polypeptide. In some aspects, the first polypeptide comprises an MM, a CM, and at least one antibody variable domain selected from the group selected from an light chain variable domain (“LVD” or “VL”) and a heavy chain variable domain (“HVD” or “VH”). In some examples, the AB (e.g., the ABs in FIGS. 2A-2D) may comprise multiple polypeptides (e.g., the AB is a complex formed by multiple polypeptides) In some aspects, the AB comprises at least two polypeptides, at least three polypeptides, at least four polypeptides, or more. In such cases, the MM may be coupled to a polypeptide of the AB via the CM and tethered to the same polypeptide via the non-alpha-carbon covalent bond. Alternatively, the MM may be coupled to a first polypeptide of the AB via the CM and tethered to a second polypeptide of the AB via the non-alpha-carbon covalent bond. The activatable antibody can have one or more polypeptides in the arrangement MM-CM-HVD or MM-CM-LVD or MM-CM-scFv, MM-CM-ScFv-Fab, MM-CM-HVD-scFv, MM-CM-LVD-scFv, MM-CM-scFv-HVD, MM-CM-scFv-LVD, HVD-CM-MM, LVD-CM-MM, scFv-CM-MM, HVD-scFv-CM-MM, LVD-scFv-CM-MM, scFv-HVD-CM-MM, MM-CM-VHH, VHH-CM-MM, or scFv-LVD-CM-MM. As used herein and unless otherwise stated, each dash (-) between the ACC components represents either a direct linkage or linkage via one or more linkers.

In some embodiments, the activatable antibody may have two polypeptides. In some examples, the activatable antibody may comprise a first polypeptide comprising a HVD and a second polypeptide comprising any one of, from an N- to C-terminal direction: MM-CM-LVD, MM-CM-scFv, MM-CM-LVD-scFv, MM-CM-scFv-LVD, LVD-CM-MM, scFv-CM-MM, LVD-scFv-CM-MM, MM-CM-VHH, VHH-CM-MM, or scFv-LVD-CM-MM. In some examples, the activatable antibody may comprise a first polypeptide comprising a LVD and a second polypeptide comprising any one of, from an N- to C-terminal direction: MM-CM-HVD, MM-CM-scFv, MM-CM-HVD-scFv, MM-CM-scFv-HVD, HVD-CM-MM, scFv-CM-MM, HVD-scFv-CM-MM, MM-CM-VHH, or scFv-HVD-CM-MM. In such cases, the MM may be tethered to the second polypeptide (i.e., the polypeptide comprising the MM) via the non-alpha-carbon covalent bond. Alternatively or additionally, the MM may be tethered to the first polypeptide (i.e., the polypeptide not comprising the MM) via the non-alpha-carbon covalent bond.

In some embodiments, the activatable antibody may have more than two polypeptides Such activatable antibody may comprise any combination of the polypeptides described above. In some embodiments, the activatable antibody may comprise four polypeptides. In some examples, in two of the polypeptides, each may comprise a HVD; in the other two polypeptides, each may comprise, from an N- to C-terminal direction: MM-CM-LVD, MM-CM-scFv, MM-CM-LVD-scFv, MM-CM-scFv-LVD, LVD-CM-MM, scFv-CM-MM, LVD-scFv-CM-MM, MM-CM-VHH, VHH-CM-MM, or scFv-LVD-CM-MM. In some examples, in two of the polypeptides, each may comprise a LVD; in the other two polypeptides, each may comprise from an N- to C-terminal direction: MM-CM-HVD, MM-CM-scFv, MM-CM-HVD-scFv, MM-CM-scFv-HVD, HVD-CM-MM, scFv-CM-MM, HVD-scFv-CM-MM, scFv-HVD-CM-MM. In these cases, the MM may be tethered to the polypeptide comprising the MM via the non-alpha-carbon covalent bond. Alternatively or additionally, the MM may be tethered to the polypeptide not comprising the MM via the non-alpha-carbon covalent bond.

In the examples described herein, the HVD and LVD may be comprised in an antibody or a fragment thereof (e.g., Fab). The activatable antibody may further comprise one or more additional components of an antibody, e.g., such as a heavy chain constant region (CH), light chain constant region (CL), hinge, Fc domain, or a combination thereof.

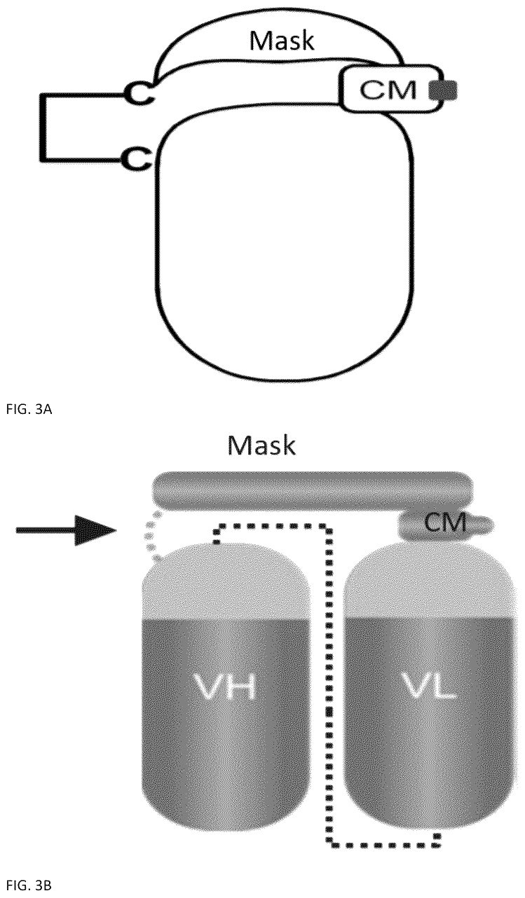

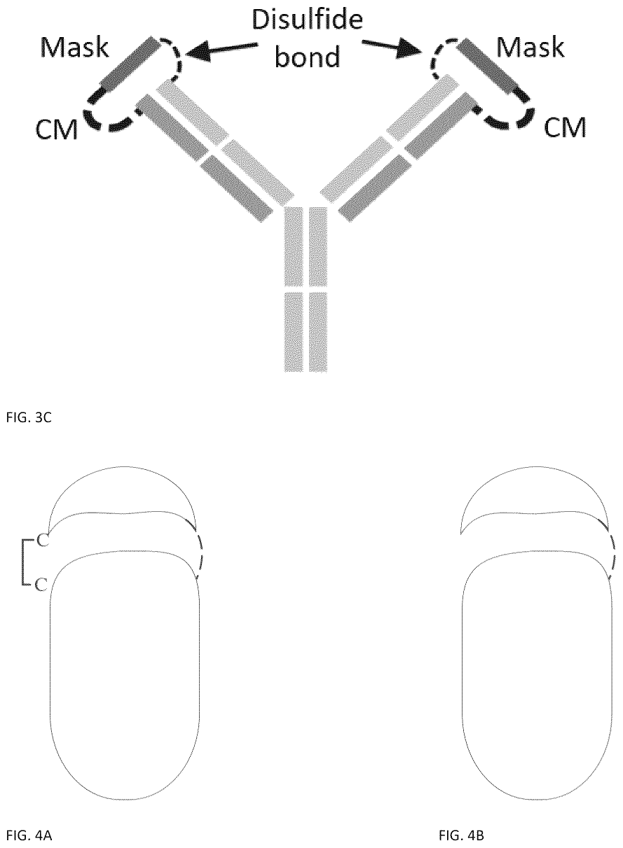

FIGS. 3A-3C show three example configurations of the activatable antibodies. FIG. 3A shows an example activatable antibody comprising a nanobody (i.e., a single domain antibody as an exemplary antibody or TB) and a MM coupled thereto via a CM. The nanobody and the MM are tethered by a non-alpha-carbon covalent bond between the mask and the nanobody.

FIG. 3B shows an activatable single chain fragment variable (scFv) comprising a scFv comprising a heavy chain variable region (V_{H}) and light chain variable region (V_{L}), and an MM coupled with the V_{L} via a CM. The MM and the scFv are also tethered by a non-alpha-carbon covalent bond between the MM and the V_{H}. Examples of activatable antibodies also include several alternative configurations of activatable scFv in FIG. 3B. In one example, the MM is coupled to the V_{L} of the scFv via a CM. The MM and the scFv are also tethered by a non-alpha-carbon covalent bond between the mask and the V_{L}. In another example, the MM is coupled to the V_{H} of the scFv via a CM. The MM and the scFv are also tethered by a non-alpha-carbon covalent bond between the MM and the V_{H}. In another example, the MM is coupled to the V_{H} of the scFv via a CM. The MM and the scFv are also tethered by a non-alpha-carbon covalent bond between the MM and the V_{L}.

FIG. 3C shows an activatable full-length antibody comprising a dimer, each monomer of the dimer comprising a heavy chain, a light chain, an MM coupled with the light chain via a CM. The MM and the activatable full-length antibody are tethered by a non-alpha-carbon covalent bond between the MM and the heavy chain. Examples of activatable antibodies also include several alternative configurations of activatable full-length antibody in FIG. 3C. In one example, the MM is coupled with the light chain via a CM. The MM and the full-length antibody are tethered by a non-alpha-carbon covalent bond between the MM and the light chain. In another example, the MM is coupled with the heavy chain via a CM. The MM and the full-length antibody are tethered by a non-alpha-carbon covalent bond between the MM and the light chain. In another example, the MM is coupled with the heavy chain via a CM. The MM and the full-length antibody are tethered by a non-alpha-carbon covalent bond between the MM and the heavy chain.

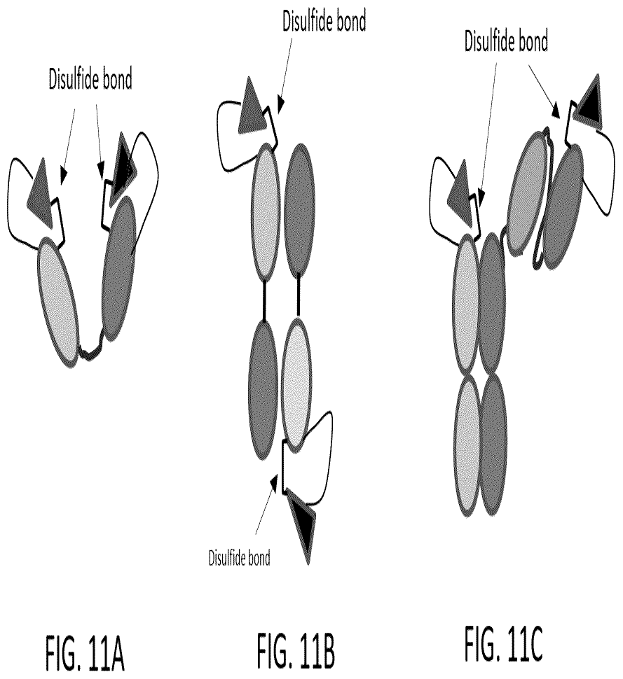

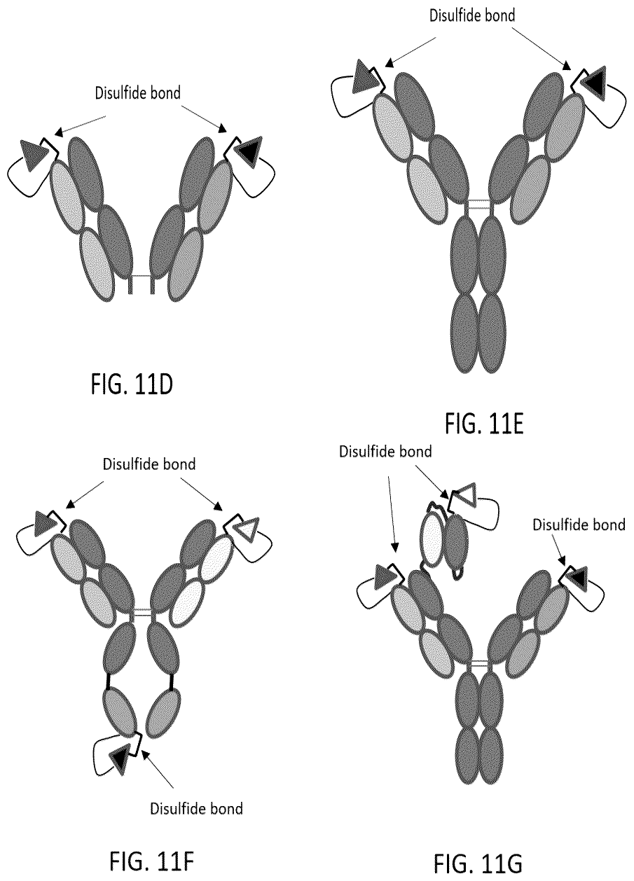

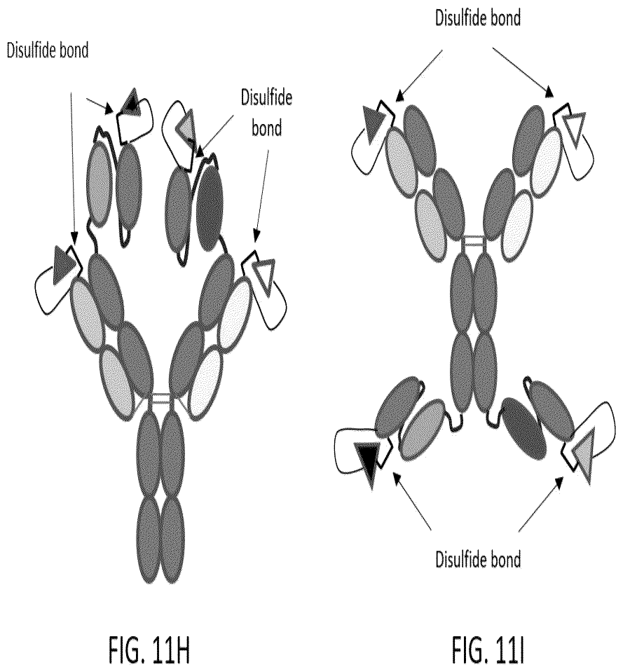

The schematics in FIGS. 2A-2D and 3A-3B are exemplified as a non-limiting proof-of-concept examples. The activatable antibodies in which an MM is dual-anchored (e.g., via a CM as well as a non-alpha-carbon covalent bond) with an AB broadly includes any type of activatable antibodies, including activatable full-length antibodies, activatable multispecific antibodies (e.g., bispecific and trispecific antibodies, including multispecific antibodies capable of crosslinking two cells such as bispecific T cell engagers (BiTEs)), and activatable antibody fragments (e.g., scFv, diabody, nanobody, Fab, etc.). Examples of dual-anchored multispecific activatable antibodies are shown in FIGS. 11A-11I. While FIGS. 11A-11I depict dual anchoring at all mask positions, the present disclosure also includes constructs comprising one or more masks that are dual-anchored and one or more other masks that are not dual-anchored (i.e., only anchored to the TB via a CM (single anchored)) The present disclosure also includes constructs comprising one or more masks that are dual-anchored and one or more TBs that are not masked. FIG. 11A shows an exemplary bispecific nanobody tandem. FIG. 11B shows an exemplary diabody comprising two scFvs. In some examples, the two scFvs may be coupled by a peptide linker. In alternative examples, the two scFvs are not coupled by any linker. FIG. 11C shows an exemplary bispecific antibody comprising a scFv and a Fab. In some examples, the bispecific antibody may be a bispecific T cell engager (BITE). The antibody in FIG. 11C may further comprise one or more Fc domains. FIG. 11D shows an exemplary bispecific F(ab′)₂. FIG. 11D shows an exemplary bispecific antibody. FIGS. 11E-11I show further examples of dual-anchored trispecific and other multispecific activatable antibodies. In dual-anchored multispecific activatable antibodies, an MM may be coupled with an AB via a CM. The MM may be tethered by one or more non-alpha-carbon covalent bonds with the AB coupled with the MM, or with another component of the activatable antibody. In some examples of multispecific activatable antibodies, all of the MMs are dual-anchored. In certain examples of multispecific activatable, only some, but not all, of the MMs are dual-anchored. The TBs in FIGS. 2A-2D may be any antigen-binding proteins, including antibodies and those described in the Target-binding proteins section below.

In some embodiments, the activatable target-binding protein (e.g., an activatable antibody) may be characterized by a reduction in its target-binding activity as compared to a control level of the target-binding activity of the AB without the MM coupled to it. For example, the activatable TB is characterized by at least a 1, 2, 4, 6, 8, 10, 20, 30, 40, 50, 60, 70, 80, 90, 100, 200, 300, 400, 500, 600, 700, 800, 900, 1000, 2000, 5000, or 10000 fold reduction in targeting binding activity as compared to the control level of the target-binding activity of the TB without the MM coupled to it.

In some embodiments, the activatable TB (e.g., activatable antibody) may be characterized by a reduction in its target-binding activity as compared to a control level of the target-binding activity of the TB with the MM coupled to it but the TB and the MM are not tethered by a non-alpha-carbon covalent bond (i.e., a single-anchored activatable antibody). For example, in some embodiments, the activatable TB is characterized by at least a 2-, 4-, 6-, 8-, 10-, 20-, 30-, 40-, 50-, 60-, 70-, 80-, 90-, 100-, 200-, 300-, 400-, 500-, 600-, 700-, 800-, 900-, 1000-, 2000-, 5000-, 10000-, 15000-, 20000-, 30000-, 40000-, or 50000-fold reduction in targeting binding activity as compared to the control level of the target-binding activity of the TB with the MM coupled to it, but the TB and the MM are not tethered by a non-alpha-carbon covalent bond. In some embodiments, the activatable TB is characterized by at least a 2-fold reduction in the targeting binding activity. In some embodiments, the activatable TB is characterized by at least a 4-fold reduction in the targeting binding activity. In some embodiments, the activatable TB is characterized by at least a 10-fold reduction in the targeting binding activity. In some embodiments, the activatable TB is characterized by at least a 500-fold reduction in the targeting binding activity. In some embodiments, the activatable TB is characterized by at least a 100-fold reduction in the targeting binding activity. In some embodiments, the activatable TB is characterized by at least a 200-fold reduction in the targeting binding activity. In some embodiments, the activatable TB is characterized by at least a 300-fold reduction in the targeting binding activity. In some embodiments, the activatable TB is characterized by at least a 500-fold reduction in the targeting binding activity. In some embodiments, the activatable TB is characterized by at least a 1000-fold reduction in the targeting binding activity. In some embodiments, the activatable TB is characterized by at least a 5000-fold reduction in the targeting binding activity. In some embodiments, the activatable TB is characterized by at least a 10000-fold reduction in the targeting binding activity. In some embodiments, the activatable TB is characterized by at least a 15000-fold reduction in the targeting binding activity. In some embodiments, the activatable TB is characterized by at least a 20000-fold reduction in the targeting binding activity. In some embodiments, the activatable TB is characterized by at least a 30000-fold reduction in the targeting binding activity

### Target-Binding Proteins

An activatable target-binding protein (e.g., activatable antibody) disclosed herein may comprise one or more target-binding proteins, i.e. proteins capable of binding to a target molecule. In some embodiments, the target-binding proteins (TBs) may be a cytokine, hormone, growth factor, or an agonist. In some embodiments, the target-binding proteins (TBs) may be antigen-binding proteins (ABs). In some embodiments, the AB may be an antibody or a fragment thereof, e.g., a monoclonal antibody, single chain antibody, Fab fragment, F(ab′)₂ fragment, single-chain variable fragment (scFv), diabody (a noncovalent dimer of scFv), single chain antibody (scab), a VHH, a domain antibody (dAb) or single domain antibody (SDA) (nanobody, e.g., single domain heavy chain antibody, single domain light chain antibody). In some embodiments, the AB may be a full-length antibody. In some embodiments, the AB may be an immunologically active fragment. In some embodiments, the AB may be an antigen-binding fragment (“Fab”). In some embodiments, the AB may be a mouse, other rodent, chimeric, humanized or fully human monoclonal antibody. The present disclosure includes structures having combinations of one or more polypeptides comprising any of the domains listed above, e.g., one or more of SDA, Fv, ScFv, Fab, scFab, VHH, and dAb, with one or more selected from SDA, Fv, scFv, Fab, VHH, scFab, and dAb.

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, bispecific, and multi-specific antibodies. One example of an antigen-binding domain is an antigen-binding domain formed by a VH-VL dimer. Additional examples of an antibody are described herein. Additional examples of an antibody are known in the art.

In some examples, the AB may be a single domain antibody (also referred to as nanobody). A single domain antibody may be an antibody fragment that is a single monomeric variable antibody domain. A single domain antibody may have similar affinity to antigens as a corresponding full-length antibody. A single domain antibody may be a Fc-tagged single domain antibody, which comprises an Fc dimer, each Fc monomer coupled with a single domain antibody.

In some embodiments, the AB may be monospecific, e.g. capable of binding to only one antigen. In some embodiments, the AB may be multispecific (e.g., bispecific or trispecific), e.g., capable of binding to multiple antigens. In some embodiments, the activatable antibody may be formulated as part of a pro-Bispecific T Cell Engager (pro-BITE) molecule or a dual-affinity retargeting antibody (DART). In some embodiments, the activatable antibody may be formulated as part of a pro-Chimeric Antigen Receptor (pro-CAR) modified T cell or other engineered receptor or other immune effector cell, such as a CAR modified NK cell. In some embodiments, the activatable antibody may be formulated as part of a pro-Chimeric Antigen Receptor (CAR) modified T cell. In some embodiments, the activatable antibody may be formulated as part of a pro-Chimeric Antigen Receptor (CAR) modified NK cell. In some embodiments, the activatable antibody may be formulated as part of a T cell bispecific antibody (TCB).

A “light chain” consists of one variable domain (VL) and one constant domain (CL). There are two different light chain types or classes termed kappa or lambda.

A “heavy chain” consists of one variable domain (VH) and three constant region domains (CH1, CH2, CH3). There are five main heavy-chain classes or isotypes, some of which have several subtypes, and these determine the functional activity of an antibody molecule. The five major classes of immunoglobulin are immunoglobulin M (IgM), immunoglobulin D (IgD), immunoglobulin G (IgG), immunoglobulin A (IgA), and immunoglobulin E (IgE). IgG is by far the most abundant immunoglobulin and has several subclasses (IgG1, 2, 3, and 4 in humans).

A “fragment antigen binding” (Fab) contains a complete light chain paired with the VH domain and the CH1 domain of a heavy chain.

A F(ab′)₂ fragment is formed when an antibody is cleaved by pepsin below the hinge region, in which case the two fragment antigen-binding domains (Fabs) of the antibody molecule remain linked. A F(ab′)₂ fragment contains two complete light chains paired with the two VH and CH1 domains of the heavy chains joined together by the hinge region.

A “fragment crystallizable” (Fc) fragment (also referred to herein as F_{C} domain) corresponds to the paired CH2 and CH3 domains and is the part of the antibody molecule that interacts with effector molecules and cells. The functional differences between heavy-chain isotypes lie mainly in the Fc fragment.

A “single chain Fv” (scFv) contains only the variable domain of a light chain (VL) linked by a stretch of synthetic peptide to a variable domain of a heavy chain (VH). The name single-chain Fv is derived from Fragment variable.

A “hinge region” or “interdomain” is flexible amino acid stretch that joins or links the Fab fragment to the Fc domain.

A “synthetic hinge region” is an amino acid sequence that joins or links a Fab fragment to an Fc domain.

“Prodomain” refers to a polypeptide that has a portion that inhibits antigen binding referred to as a masking peptide (MM) and a portion containing a protease cleavable substrate referred to as a cleavable peptide (CM) that when linked to a target-binding protein (TB), an antibody, antigen binding fragment thereof, or antigen-binding domain (AB) functions to inhibit antigen binding by the TB or AB. The prodomain may include a linker peptide (L1) between the MM and the CM. The prodomain may also include a linker peptide (L2) at the prodomain's carboxyl terminus to facilitate the linkage of the prodomain to the antibody. In certain embodiments, a prodomain comprises one of the following formulae (wherein the formula below represents an amino acid sequence in an N- to C-terminal direction): (MM)-(CM), (MM)-L1-(CM), (MM)-(CM)-L2, or (MM)-L1-(CM)-L2.

The TB (e.g., an AB) specifically binds to a target. As used herein, the terms “specific binding,” “immunological binding,” and “immunological binding properties” refer to the noncovalent interactions of the type that occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides may be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). A TB of the present disclosure may specifically bind to the target with a binding constant (K_{d}) of ≤1 μM. In some embodiments, TB of the present disclosure may specifically bind to the target with a binding constant (K_{d}) of ≤100 nM. In some embodiments, TB of the present disclosure may specifically bind to the target with a binding constant (K_{d}) of ≤10 nM. In some embodiments, TB of the present disclosure may specifically bind to the target with a binding constant (K_{d}) of ≤100 pM to about 1 pM, as measured by assays such as radioligand binding assays or similar assays known to those skilled in the art.

The target of the TB (e.g., AB) may be a protein or other types of molecules. Example classes of targets of a TB include cell surface receptors and secreted binding proteins (e.g., growth factors), soluble enzymes, structural proteins (e.g. collagen, fibronectin) and the like. In some examples, the target of a TB may be a protein associated with a disease (e.g., cancer) in a subject.

In some embodiments, the activatable target-binding protein or activatable antibody may comprise a serum half-life extending moiety (e.g., polypeptides that bind serum proteins, such as immunoglobulin (e.g., IgG) or serum albumin (e.g., human serum albumin (HSA)). The half-life extending moiety may be coupled with the TB.

In some examples, the half-life extending moiety may be a fragment crystallizable region (Fc) region of an antibody. Other examples of half-life extending moieties include hexa-hat GST (glutathione S-transferase) glutathione affinity, Calmodulin-binding peptide (CBP), Strep-tag, Cellulose Binding Domain, Maltose Binding Protein, S-Peptide Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, and VSV Epitope.

In some embodiments, the serum half-life of the activatable target-binding protein or activatable antibody may be longer than that of the corresponding protein (e.g., an activatable antibody does not have the half-life extending moiety), e.g., the pK of the activatable antibody is longer than that of the corresponding antibody. In some embodiments, the serum half-life of the activatable target-binding protein or activatable antibody is similar to that of the corresponding antibody. In some embodiments, the serum half-life of the activatable target-binding protein (e.g., an activatable antibody) is at least 15 days, 12 days, 11 days, 10 days, 9 days, 8 days, 7 days, 6 days, 5 days, 4 days, 3 days, 2 days, 1 day, 20, 18 hours, 16 hours, 14 hours, 12 hours, 10 hours, 8 hours, 6 hours, 4 hours, 3 hours, 2 hours, or 1 hour when administered to an organism.

### Masking Moieties (MMs)

A masking moiety in an activatable macromolecule “masks” or reduces or otherwise inhibits the binding of the activatable macromolecule to its target and/or epitope. In some embodiments, the coupling or modifying of a target-binding protein (TB) (e.g., an AB or other therapeutic or diagnostic protein) with a MM can inhibit the ability of the TB to specifically bind its target and or epitope by means of inhibition known in the art (e.g., without limitation, structural change and competition for antigen-binding domain). In some embodiments, the coupling or modifying of a TB with a MM can effect a structural change that reduces or inhibits the ability of the TB to specifically bind its target and or epitope. In some embodiments, the coupling or modifying of a protein comprising an antigen-binding domain with a MM sterically blocks, reduces or inhibits the ability of the antigen-binding domain to specifically bind its target and or epitope.

The activatable target-binding proteins (for example activatable antibodies) herein may comprise one or more mask moieties (MMs), which is capable of interfering with the binding of the target-binding protein (e.g., AB) to the target. In general, a MM may be coupled to a target-binding protein (e.g., AB) by a CM and optionally one or more linkers described herein. Further, a MM may additionally be tethered to the activatable target-binding protein (or antibody) as described herein to form an activatable dual-anchored masked target-binding protein. In some embodiments, the MM prevents the activtable TB from target binding; but when the molecule is activated (when the CM is cleaved by a protease), the MM does not substantially or significantly interfere with the target binding protein's binding to the target.

In some embodiments, an MM may interact with the AB (or other desired protein), thus reducing or inhibiting the interaction between the target-binding protein (e.g., AB) and its binding partner. In some embodiments, the MM may comprise at least a partial or complete amino acid sequence of a naturally occurring binding partner of the target-binding protein (e.g., AB). For example, the MM may be a fragment of a naturally occurring binding partner. The fragment may retain no more than 95%, 90%, 80%, 75%, 70%, 60%, 50%, 40%, 30%, 25%, or 20% nucleic acid or amino acid sequence homology to the naturally occurring binding partner. In some embodiments, the MM may be a cognate peptide of the target-binding protein (e.g., AB). For example, the MM may comprise a sequence of the target-binding protein's (e.g., AB's) epitope or a fragment thereof. The term “naturally occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally occurring.

In some embodiments, the MM may comprise an amino acid sequence that is not naturally occurring or does not contain the amino acid sequence of a naturally occurring binding partner or target protein. In certain embodiments, the MM is not a natural binding partner of the target-binding protein (e.g., AB). The MM may be a modified binding partner for the target-binding protein (e.g., AB) which contains amino acid changes that decrease affinity and/or avidity of binding to the target-binding protein (e.g., AB). In some embodiments the MM may contain no or substantially no nucleic acid or amino acid homology to the AB's natural binding partner. In other embodiments the MM is no more than 5%, 10%, 15%, 20%, 25%, 30%, 35%, 40%, 45%, 50%, 55%, 60%, 65%, 70%, 75%, or 80% similar to the natural binding partner of the target-binding protein (e.g., AB).

In some embodiments, the MM may not specifically bind to the AB (or other activatable protein), but interfere with target-binding protein's (e.g., AB's) binding to its binding partner through non-specific interactions such as steric hindrance. For example, the MM may be positioned in the activatable target-binding protein such that the tertiary or quaternary structure of the activatable target-binding protein allows the MM to mask the target-binding protein through charge-based interaction, thereby holding the MM in place to interfere with binding partner access to the target-binding protein.

In some embodiments, the MM may have a dissociation constant for binding to the target-binding protein (e.g., AB) that is no more than the dissociation constant of the target-binding protein to the target. In some embodiments, the MM may not interfere or compete with the target-binding protein for binding to the target after cleavage of the CM.

The structural properties of the MMs may be selected according to factors such as the minimum amino acid sequence required for interference with protein binding to target, the target protein-protein binding pair of interest, the size of the target-binding protein, the presence or absence of linkers, and the like.

In some embodiments, the MM may be unique for the coupled target-binding protein. Examples of MMs include MMs that were specifically screened to bind a binding domain of the target-binding protein or fragment thereof (e.g., affinity masks). Methods for screening MMs to obtain MMs unique for the target-binding protein and those that specifically and/or selectively bind a binding domain of a binding partner/target are provided herein and can include protein display methods.

As used herein, the term “masking efficiency” or “ME” refers to the activity (e.g., EC50) of the activatable target-binding protein (e.g., activatable AB) divided by the activity of a control target-binding protein (e.g., antibody), wherein the control target-binding protein (e.g., control antibody) may be either cleavage product of the activatable target-binding protein (e.g., activatable antibody) or the target-binding protein (e.g., antibody) or fragment thereof used as the target-binding protein of the activatable target-binding protein. An activatable target-binding protein having a reduced level of a targeting binding (or antibody) activity may have a masking efficiency that is greater than 10. In some embodiments, the activatable target-binding proteins (e.g. activatable antibodies) described herein may have a masking efficiency that is greater than 10, 100, 1000, 5000, 10,000, or 15,000.

In some embodiments, the MM may be a polypeptide of about 2 to 50 amino acids in length. For example, the MM may be a polypeptide of from 2 to 40, from 2 to 30, from 2 to 20, from 2 to 10, from 5 to 15, from 10 to 20, from 15 to 25, from 20 to 30, from 25 to 35, from 30 to 40, from 35 to 45, from 40 to 50 amino acids in length. For example, the MM may be a polypeptide with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some examples, the MM may be a polypeptide of more than 50 amino acids in length, e.g., 100, 200, 300, 400, 500, 600, 700, 800, or more amino acids.

In some embodiments, when an MM is tethered to the target-binding protein (e.g., AB) to which it is coupled, in the presence of the target of the target-binding protein, there may be no binding or substantially no binding of the target-binding protein to the target, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the target-binding protein to its target, as compared to the binding of the target-binding protein coupled to an MM but not tethered with the MM (i.e., not dual-anchored), for at least 0.1, 0.5, 1, 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or greater when measured in vivo or in a masking efficiency assay, or in an in vitro immunoabsorbant assay, e.g., as described in US20200308243A1.

The binding affinity of the TB (e.g., AB) towards the target or binding partner when the TB (e.g., AB) is tethered to an MM coupled with the TB (e.g., AB) may be at least 5, 10, 25, 50, 100, 250, 500, 1,000, 2,500, 5,000, 10,000, 50,000, 100,000, 500,000, 1,000,000, 5,000,000, 10,000,000, or 50,000,000 times lower than the binding affinity of the TB (e.g., AB) towards its binding partner when the TB (e.g., AB) is not coupled to a MM, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the TB (e.g., AB) towards its binding partner when not coupled to an MM.

The binding affinity of the TB (e.g., AB) towards the target or binding partner when the TB (e.g., AB) is tethered to an MM coupled with the TB (e.g., AB) may be at least 5, 10, 25, 50, 100, 250, 500, 1,000, 2,500, 5,000, 10,000, 50,000, 100,000, 500,000, 1,000,000, 5,000,000, 10,000,000, or 50,000,000 times lower than the binding affinity of the TB (e.g., AB) towards its binding partner when the TB (e.g., AB) is cleaved from the MM by a protease.

The binding affinity of the TB (e.g., AB) towards the target or binding partner when the TB (e.g., AB) is tethered to an MM coupled with the TB (e.g., AB) may be at least 5, 10, 25, 50, 100, 250, 500, 1,000, 2,500, 5,000, 10,000, 50,000, 100,000, 500,000, 1,000,000, 5,000,000, 10,000,000, or 50,000,000 times lower than the binding affinity of the TB (e.g., AB) towards its binding partner when the TB (e.g., AB) is not coupled and not tethered to a MM, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the TB (e.g., AB) towards its binding partner when not coupled and not tethered to an MM.

The dissociation constant (K_{d}) of the MM towards the TB (e.g., AB) to which it is coupled and tethered (dual-anchored), may be greater than the K_{d} of the TB (e.g., AB) towards the target. The K_{d} of the MM towards the TB (e.g., AB) may be at least 5, 10, 25, 50, 100, 250, 500, 1,000, 2,500, 5,000, 10,000, 100,000, 1,000,000 or even 10,000,000 times greater than the K_{d} of the TB (e.g., AB) towards the target. Conversely, the binding affinity of the MM towards the TB (e.g., AB) may be lower than the binding affinity of the TB (e.g., AB) towards the target. The binding affinity of MM towards the TB (e.g., AB) may be at least 5, 10, 25, 50, 100, 250, 500, 1,000, 2,500, 5,000, 10,000, 100,000, 1,000,000 or even 10,000,000 times lower than the binding affinity of the TB (e.g., AB) towards the target.

In some embodiments, the MMs may contain genetically encoded or genetically non-encoded amino acids. Examples of genetically non-encoded amino acids are but not limited to D-amino acids, β-amino acids, and γ-amino acids. In specific embodiments, the MMs contain no more than 50%, 40%, 30%, 20%, 15%, 10%, 5% or 1% of genetically non-encoded amino acids.

In some embodiments, once uncoupled from the TB and in a free state, the MM may have a biological activity or a therapeutic effect, such as binding capability. For example, the free peptide may bind with the same or a different binding partner. In certain embodiments the free MM (e.g., MM not coupled or tethered with the TB) may exert a therapeutic effect, providing a secondary function to the compositions disclosed herein. In some embodiments, once uncoupled from the TB and in a free state, the MM may advantageously not exhibit biological activity. For example, in some embodiments, the MM in a free state does not elicit an immune response in the subject.

The TB and the MM may comprise one or more cysteine residues capable of forming non-alpha-carbon covalent bond(s) between the TB and the MM. In some embodiments, the one or more non-alpha-carbon covalent bonds may be formed between sulfur atoms of cysteine or other amino acid residues containing a sulfur atom. Such residues may occur naturally in the activatable TB (e.g., in the TB and the MM) or may be incorporated into the activatable TB by site-directed mutagenesis, chemical conversion, or mis-incorporation of non-natural amino acids.

In some embodiments, the cysteine residues may be at a position that provides for a conformationally constrained activatable TB, but that, following CM cleavage, the MM does not substantially or significantly interfere with target binding of the activated TB.

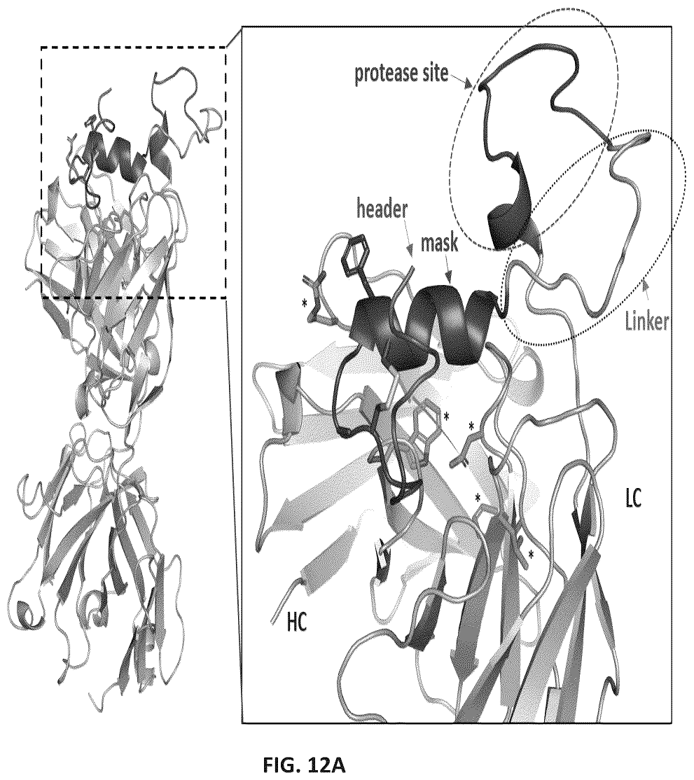

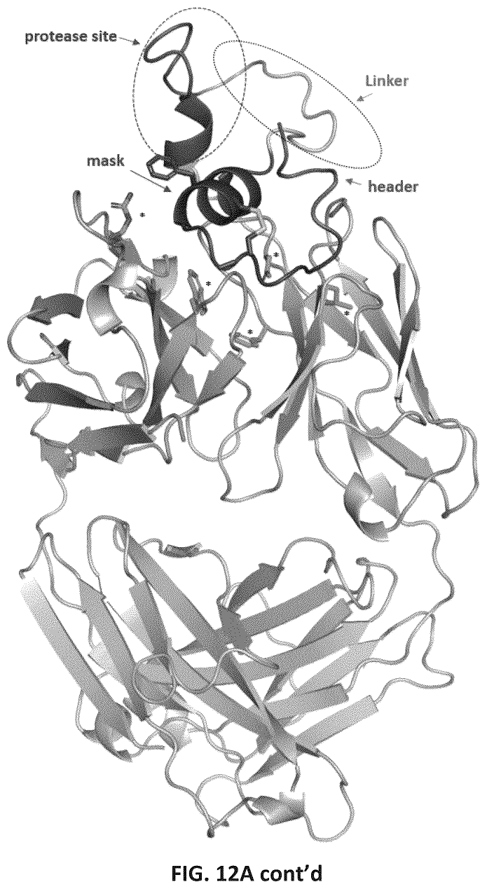

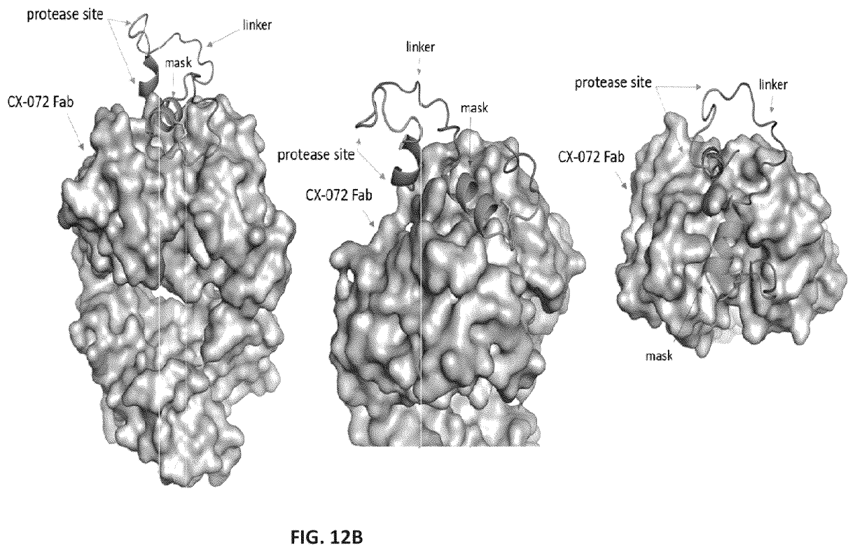

The positions of the cysteine residues in the activatable target-binding proteins may be determined based on the structure (e.g., the crystal structure, or other structure models based on other techniques such as NMR, spectroscopic, or computational methods) of the activatable target-binding proteins or components thereof. Any of a variety of homology-based computation protein models may be used to generate a three-dimensional structure of a target-binding protein, either with or without an MM, including, for example, Rosetta modeling software (rosettacommons.org), Discovery Studio (Dassault Systemes BIOVIA), BioLuminate, PIPER, Prime (Schrodinger, Inc.), AlphaFold Colab (Google, Inc.), SWISSMODELER, and the like. An example of a three-dimensional structure obtained using Discovery Studio software, is that of an activatable target binding protein (activatable anti-PDL1 antibody) depicted in FIGS. 12A-12B. For example, the region where the MM interacts with the TB may be determined and cysteine residues (naturally occurring or introduced) in the region may be used to form the non-alpha-carbon covalent bond tethering the TB and the MM. Generally, the Cα atoms of disulfide bonded cysteine residues are in the 3.0-7.5 Å range. Thus, as a first approximation, MM and TB residues with Cα distances in that range would be good candidates for cysteine mutation. Any of a variety of disulfide prediction programs, including, for example, MODIP (Dani, Ramakrishnan, Varadarajan 2003), Disulfide by Design (Craig & Dombkowski, 2013), and SSbondPre (Gao, Dong, Li, Liu & Liu, 2020) may be used to further identify MM and TB residues with a high likelihood to form disulfide bonds.

In some embodiments, the non-alpha-carbon covalent bond, e.g., a disulfide bond tethering the MM and the TB (e.g., AB) may be formed by a first cysteine and a second cysteine. In one example, the first cysteine is within the MM and the second cysteine is within the TB. In another example, the first cysteine is within a peptide coupled to the MM and the second cysteine is within the TB. In another example, the first cysteine is within the MM and the second cysteine is within a peptide coupled to the TB. In such examples, the peptide coupled to the MM or the TB may be a linker or peptide. In one example, the peptide coupled to the MM or the TB may be a leader peptide, which is a peptide position adjacent to a terminus (e.g., the N-terminus or C terminus) of the MM or TB. The leader peptide may be positioned between a signal peptide and the MM or TB.

In some embodiments, one or more cysteine(s) forming the disulfide bond with the MM may be naturally occurring cysteine(s) in the activatable target binding proteins. In some embodiments, one or more of the cysteines forming the disulfide bond may be engineered into the activatable TBs (e.g., ABs).

Suitable MMs may be identified and/or further optimized through a screening procedure from a library of candidate activatable TBs having variable MMs. For example, a TB and a CM may be selected to provide for a desired enzyme/target combination, and the amino acid sequence of the MM can be identified by the screening procedure described below to identify an MM that provides for a activatable phenotype. For example, a random peptide library (e.g., of peptides comprising 2 to 40 amino acids or more) may be used in the screening methods disclosed herein to identify a suitable MM.

In some embodiments, MMs with specific binding affinity for a TB (e.g., an AB) may be identified through a screening procedure that includes providing a library of peptide scaffolds consisting of candidate MMs wherein each scaffold is made up of a transmembrane protein and the candidate MM. The library may then be contacted with an entire or portion of a protein such as a full length protein, a naturally occurring protein fragment, or a non-naturally occurring fragment containing a protein (also capable of binding the binding partner of interest), and identifying one or more candidate MMs having detectably bound protein. The screening may be performed by one more rounds of magnetic-activated sorting (MACS) or fluorescence-activated sorting (FACS), as well as determination of the binding affinity of MM towards the AB and subsequent determination of the masking efficiency, e.g., as described in WO2009025846 and US20200308243A1, which are incorporated herein by reference in their entireties. In some embodiments, an MM may be selected for use with a specific antibody or antibody fragment. Additional suitable MMs are disclosed in WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, WO2016014974, and WO2016118629.

### Cleavable Moieties (CMs)

The activatable target-binding protein may comprise one or more cleavable moieties (CMs) as defined above.

In some embodiments, the activatable TB may comprise a CM between the TB (e.g., AB) and the MM. The CM and the TB of the activatable target-binding proteins (e.g., ABs) may be selected so that the TB comprises a binding moiety for a given target, and the CM comprises a substrate for one or more proteases, where the one or more proteases is/are co-localized with the target in a tissue (e.g., at a treatment site or diagnostic site in a subject). In some embodiments, the activatable TBs may find particular use where, for example, one or more proteases capable of cleaving a site in the CM, is present at relatively higher levels in target-containing tissue of a treatment site or diagnostic site than in tissue of non-treatment sites (for example in healthy tissue).

In some embodiments, the CMs herein may comprise substrates for proteases that have been reported in a cancer, or in a number of cancers. See, e.g., La Roca et al., British J. Cancer 90(7):1414-1421, 2004. Substrates suitable for use in the CM components employed herein include those which are more prevalently found in cancerous cells and tissue. Thus, in certain embodiments, the CM may comprise a substrate for a protease that is more prevalently found in diseased tissue associated with a cancer. Examples of the cancers include gastric cancer, breast cancer, osteosarcoma, esophageal cancer, breast cancer, a HER2-positive cancer, Kaposi sarcoma, hairy cell leukemia, chronic myeloid leukemia (CML), follicular lymphoma, renal cell cancer (RCC), melanoma, neuroblastoma, basal cell carcinoma, cutaneous T-cell lymphoma, nasopharyngeal adenocarcinoma, ovarian cancer, bladder cancer, BCG-resistant non-muscle invasive bladder cancer (NMIBC), endometrial cancer, pancreatic cancer, non-small cell lung cancer (NSCLC), colorectal cancer, esophageal cancer, gallbladder cancer, glioma, head and neck carcinoma, uterine cancer, cervical cancer, or testicular cancer, and the like. In some embodiments, the CM components comprise substrates for protease(s) that is/are more prevalent in tumor tissue. For example, the protease(s) may be produced by a tumor in a subject.

In some embodiments, the activatable TB may comprise a first CM (CM1) between the MM and the TB (e.g., AB) and a second CM (CM2) so that the MM can be completely dissociated from the TB by cleavage of the CMs. In some examples, the first and the second CMs may comprise the substrates of the same protease. In some examples, the first and the second CMs may comprise the substrates of different proteases. In some examples, the first and the second CMs may have the same sequence. In some examples, the first and the second CMs may have different sequences.

The second CM (CM2) may be at a position in the activatable TB where its cleavage facilitates dissociation of the MM from the TB. In some embodiments, the CM2 may be positioned between the MM and the non-alpha-carbon covalent bond. In some embodiments, the second CM may be within the MM and up to 10 amino acids away from the non-alpha-carbon covalent bond. In some embodiments, the second CM may be within the TB and up to 10 amino acids away from the non-alpha-carbon covalent bond. The second CM may be positioned adjacent to a cysteine forming the non-alpha-carbon covalent bond (e.g., 0 amino acid between the second CM and the cysteine). Alternatively, the second CM may be positioned up to 10, 9, 8, 7, 6, 5, 4, 3, 2, or 1 amino acid(s) away from a cysteine forming the non-alpha-carbon covalent bond, e.g., disulfide bond. The activatable target-binding protein may have enhanced masking efficiency due to the tethering between the TB and MM to minimize the toxicities and effects caused by target binding outside the activating environment, and can be sufficiently activated by a protease to provide desired activity (e.g., therapeutic effects, target detection, etc.).

Suitable CMs for use in the activatable TB herein include any of the protease substrates that are known the art. In some examples, the CM may comprise a substrate of a serine protease (e.g., u-type plasminogen activator (uPA, also referred to as urokinase), a matriptase (also referred to herein as MT-SP1 or MTSP1). In some examples, the CM may comprise a substrate of a matrix metalloprotease (MMP). In some examples, the CM may comprise a substrate of cysteine protease (CP) (e.g., legumain).

In some embodiments, the CM may comprise a substrate for a disintegrin and a metalloproteinase (ADAM) or a disintegrin and a metalloproteinase with a thrombospondin motifs (ADAMTS) (e.g., ADAM8, ADAM9, ADAM10, ADAM12, ADAM15, ADAM17/TACE, ADEMDEC1, ADAMTS1, ADAMTS4, ADAMTS5), an aspartate protease (e.g. BACE, Renin), an aspartic cathepsin (e.g., Cathepsin D, Cathepsin E), Caspase (e.g., Caspase 1, Caspase 2, Caspase 3, Caspase 4, Caspase 5, Caspase 6, Caspase 7, Caspase 8, Caspase 9, Caspase 10, Caspase 14), cysteine cathepsin (e.g., Cathepsin A, Cathepsin B, Cathepsin C, Cathepsin G, Cathepsin K, Cathepsin L, Cathepsin S, Cathepsin V/L2, Cathepsin X/Z/P), a cysteine proteinase (e.g., Cruzipain, Legumain, Otubain-2), a Chymase, DESC1, DPP-4, FAP, an Elastase, FVIIa, FiXA, FXa, FXIa, FXIIa, Granzyme B, Guanidinobenzoatase, Hepsin, HtrA1, Human a Neutrophil Elastase, a KLK (e.g., KLK4, KLK5, KLK6, KLK7, KLK8, KLK10, KLK11, KLK13, KLK14), a metallo proteinase (e.g., Meprin, Neprilysin, PSMA, BMP-1), Lactoferrin, Marapsin, Matriptase-2, MT-SP1/Matriptase, NS3/4A, PACE4, Plasmin, PSA, an MMP (e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27), TMPRSS2, TMPRSS3, TMPRSS4, tPA, Thrombin, Tryptase, and uPA.

In some embodiments, the protease substrate in the CM may comprise a polypeptide sequence that is not substantially identical (e.g., no more than 90%, 80%, 70%, 60%, or 50% identical) to any polypeptide sequence that is naturally cleaved by the same protease.

In some embodiments, CM may be or comprise a sequence of LSGRSDNH (SEQ ID NO. 214) or PLGLAG (SEQ ID NO. 17). In some embodiments, the CM may be or comprise a sequence of encompassed by the consensus of sequence of any one of SEQ ID NOs: 317-327, 329-335, 340-347, 352-363, 371-378, 394-401, 410-419, 425-433, 436-449, 453-456, 458-469, 473, 475-482, 485-495 disclosed in WO2015048329, which is incorporated by reference herein in its entirety, and SEQ ID NOs: 1-162, 268-306 disclosed in WO2015116933, which is incorporated by reference herein in its entirety.

In some embodiments, the CM may be or comprise a sequence of any one of SEQ ID NOs. 14-52, 126-154, 159, 315-316, 328, 336-339, 348-351, 364-370, 379-393, 402-409, 420-424, 434-435, 450-452, 457, 470-472, 474, 483, 484 disclosed in WO2015048329, SEQ ID NOs: 163-267, 307-384, 402-445, 665-683 disclosed in WO2015116933, SEQ ID NOs: 20-21, 411, 480-482, 351-369, 18, 71, 370-380, 412-415, 468, 547-554, 319-346 disclosed in WO2016118629, which is incorporated by reference herein in its entirety, and SEQ ID NOs: 1-16, 50-56, 60-63, 20, 70-76, 78-115, 120-128, 130-132, 135-140, 141, 152, 21-23, 17-19, 25-43 disclosed in WO2020118109, which is incorporated by reference herein in its entirety. In some examples, the CM of a cysteine protease may be or comprise the sequence of AAN, SAN, or GPTN (SEQ ID NO: 301). Examples of CMs also include those described in WO 2010/081173, WO2021207669, WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, WO2016014974, which are incorporated herein by reference in their entireties.

In some embodiments, the CM may be or comprise a sequence or encompassed by the consensus of sequence of any one of the sequences in the table below.

| | SEQ | | SEQ |
|---|---|---|---|
| | ID | | ID |
| Sequences | NO | Sequences | NO |
| AANALAHGLF | 32 | HMMQYARH | 33 |
| AANL | 34 | HTGRSGAL | 35 |
| AANLGSGGSS | 36 | HVPRQ | 37 |
| AAPRS | 38 | HVPRQV | 39 |
| AAPRSF | 40 | HVPRQVAPRSF | 41 |
| AARGPAIH | 42 | HVPRQVLSGRS | 43 |
| AAYHLVSQ | 44 | HVPRQVLSGRSAN | 45 |
| AFPDMRSVRS | 46 | HWHLGPPT | 47 |
| AFQALRM | 48 | IANLLSMV | 49 |
| AFRHLR | 50 | IDGR | 51 |
| AGLGISST | 52 | IEGR | 53 |
| AGLGVVER | 54 | ILNLLSMV | 55 |
| AGPR | 56 | ILPRSPAF | 57 |
| AHGL | 58 | IPFSWSRF | 59 |
| AHGLF | 60 | IQNLLSMV | 61 |
| AHQALRM | 62 | ISSGL | 63 |
| AIPRVRLFDV | 64 | ISSGLL | 65 |
| ALAHG | 66 | ISSGLLS | 67 |
| ALAHGL | 68 | ISSGLLSGRSANI | 69 |
| ALAHGLF | 70 | ISSGLLSGRSANP | 71 |
| ALAHGLFAPRSF | 72 | ISSGLLSGRSANPRG | 73 |
| ALAHGLFSGRSAN | 74 | ISSGLLSGRSDDH | 75 |
| ALAHGLPTFVHL | 76 | ISSGLLSGRSDIH | 77 |
| ALGLLRLP | 78 | ISSGLLSGRSDNH | 79 |
| ALPSVKMVSE | 80 | ISSGLLSGRSDNI | 16 |
| ALRAP | 81 | ISSGLLSGRSDNP | 82 |
| ANQALRM | 83 | ISSGLLSGRSDQH | 84 |
| ANQALRMA | 85 | ISSGLLSGRSDTH | 86 |
| APPLVKSMVV | 87 | ISSGLLSGRSDYH | 88 |
| APPSFKLVNA | 89 | ISSGLLSGRSGNH | 90 |
| APRS | 91 | ISSGLLSGRSNI | 92 |
| APRSALAHGLF | 93 | ISSGLLSGRSNIG | 94 |
| APRSF | 95 | ISSGLLSGRSNIGS | 96 |
| AQFVLTEG | 97 | ISSGLLSS | 98 |
| AQNLLGMV | 99 | ISSGLLSSGGSGGSLSGRS | 100 |
| | | DNH | |
| ARGP | 101 | ISSGLLSSGGSGGSLSGRS | 102 |
| | | GNH | |
| ARGPS | 103 | ISSGLSS | 104 |
| ARGPSF | 105 | IVSRSA | 106 |
| ARGPSFK | 107 | KGLTGRSDRHQA | 108 |
| ASGLLRFP | 109 | KGPKVKVVTL | 110 |
| ASPTMKTVGL | 111 | KNLYGRSENNGN | 112 |
| AVGLLAPP | 113 | KRMPVQFL | 114 |
| AVGLLAPPGGLSGRSANI | 115 | LAAPLGLL | 116 |
| AVGLLAPPGGLSGRSANP | 117 | LAHG | 118 |
| AVGLLAPPGGLSGRSDDH | 119 | LAHGL | 120 |
| AVGLLAPPGGLSGRSDIH | 121 | LAHGLF | 122 |
| AVGLLAPPGGLSGRSDNH | 123 | LAPLGLQRR | 124 |
| AVGLLAPPGGLSGRSDNI | 125 | LARAG | 126 |
| AVGLLAPPGGLSGRSDNP | 127 | LARAGI | 128 |
| AVGLLAPPGGLSGRSDQH | 129 | LARAGL | 130 |
| AVGLLAPPGGLSGRSDTH | 131 | LKAAPRWA | 132 |
| AVGLLAPPGGLSGRSDYH | 133 | LKAAPRWF | 134 |
| AVGLLAPPGGLSGRSNI | 135 | LKAAPVWA | 136 |
| AVGLLAPPGGLSGRSNIG | 137 | LKAAPVWF | 138 |
| AVGLLAPPGGLSGRSNIG | 139 | LKGRSYYY | 140 |
| S | | | |
| AVGLLAPPGGTSTSGRSA | 141 | LLAPSHRA | 142 |
| NPRG | | | |
| AVGLLAPPSGRSANPRG | 143 | LLEALRAL | 144 |
| AVGLLAPPTSGRSANPRG | 145 | LLESLRAL | 146 |
| AVPKVRVVPE | 147 | LLLPAHGG | 148 |
| CGPPLGR | 149 | LLLPLLGS | 150 |
| CSPPLGR | 151 | LLNALRAL | 152 |
| DEVDGSGGSS | 153 | LLNSLRAL | 154 |
| DEXXXC(A/S) | | LLQALRAL | 156 |
| DISHWRRS | 157 | LLQSLRAL | 158 |
| DLAHPLL | 159 | LLSALRAL | 160 |
| DLPLVKSLPS | 161 | LLSSLRAL | 162 |
| DLXXT(A/S) | | LNGRSDNH | 164 |
| DRLSGRSANHKK | 165 | LPAGLLL | 166 |
| DRLSGRSDNHKK | 167 | LPAGLLLR | 168 |
| DRPEMKSLSG | 169 | LPAHLVLL | 170 |
| DRPKVKTMDF | 171 | LPAHLVLV | 172 |
| DVAQFVLT | 173 | LPGGLSPW | 174 |
| DVPPMKTLRP | 175 | LPSHLVLL | 176 |
| DWLYWMGI | 177 | LPSHLVLV | 178 |
| DWLYWMSI | 179 | LPTFV | 180 |
| DWLYWPGI | 181 | LPTFVH | 182 |
| DWLYWPSI | 183 | LPTFVHL | 184 |
| EAPKVKALPK | 185 | LRSGW | 186 |
| EHPRVKVVSE | 187 | LSGR | 188 |
| EKPRMKLFQG | 189 | LSGRS | 190 |
| EPQALAMS | 191 | LSGRSA | 192 |
| EQPEVKMVKG | 193 | LSGRSALAHGLF | 194 |
| ERPGVKSLVL | 195 | LSGRSAN | 196 |
| ESLPVVAV | 197 | LSGRSANI | 198 |
| ESPVMKSMAL | 199 | LSGRSANP | 200 |
| ESRRW | 201 | LSGRSD | 202 |
| ESRRWM | 203 | LSGRSDD | 204 |
| ESRRWMP | 205 | LSGRSDDH | 206 |
| ETPSVKTMGR | 207 | LSGRSDI | 208 |
| ETPSVKTMGRSS | 209 | LSGRSDIH | 210 |
| FPRPLGITGL | 211 | LSGRSDN | 212 |
| FRLLDWQW | 213 | LSGRSDNH | 214 |
| GCGPPLGR | 215 | | |
| GCSPPLGR | 217 | LSGRSDNHGGAVGLLAPP | 218 |
| GFPHMKTFQH | 219 | LSGRSDNHGGSGGSISSGL | 220 |
| | | LSS | |
| GFPHMKTFQHSS | 22 | LSGRSDNHGGSGGSQNQ | 222 |
| | | ALRMA | |
| GGGPPLGR | 223 | LSGRSDNHGGVHMPLGF | 224 |
| | | LGP | |
| GGPPLGR | 225 | LSGRSDNI | 226 |
| GGQPSGMWGW | 227 | LSGRSDNP | 228 |
| GGSIDGR | 229 | LSGRSDQ | 230 |
| GGSPPLGR | 23 | LSGRSDQH | 232 |
| GGWHTGRN | 233 | LSGRSDT | 234 |
| GIAGQ | 235 | LSGRSDTH | 236 |
| GLGTPRGLFA | 237 | LSGRSDY | 238 |
| GLPTFV | 239 | LSGRSDYH | 240 |
| GLPTFVH | 241 | LSGRSENH | 242 |
| GLPTFVHL | 243 | LSGRSG | 244 |
| GLPTFVHLPRQV | 245 | LSGRSGN | 246 |
| GLSGRSDNHGSS | 247 | LSGRSGNH | 248 |
| GPEGLRVG | 249 | LSGRSGNHGGSGGSISSGL | 250 |
| | | LSS | |
| GPLGIAGI | 251 | LSGRSGNHGGSGGSQNQ | 252 |
| | | ALRMA | |
| GPLNGRSDNHKA | 253 | LSGRSGNP | 254 |
| GPLNGRSDNHKK | 255 | LSGRSVTQ | 256 |
| GPLNGRSDNHKR | 257 | LSQARWRK | 258 |
| GPLNGRSDNHQA | 259 | LTFPTYIF | 260 |
| GPLNGRSDNHQK | 261 | LTFPTYWF | 262 |
| GPLNGRSDNHQR | 263 | LTGRSDRH | 264 |
| GPLNGRSDNHRA | 265 | LTGRSGA | 266 |
| GPLNGRSDNHRK | 267 | LTGRSGA | 268 |
| GPLNGRSDNHRR | 269 | LYAAPRWA | 270 |
| GPLSGRSDNHKA | 27 | LYAAPRWF | 272 |
| GPLSGRSDNHKK | 273 | LYAAPVWA | 274 |
| GPLSGRSDNHKR | 275 | LYAAPVWF | 276 |
| GPLSGRSDNHQA | 277 | LYGRSENN | 278 |
| GPLSGRSDNHQK | 279 | MDAFLESS | 280 |
| GPLSGRSDNHQR | 281 | MGLFSEAG | 282 |
| GPLSGRSDNHRA | 283 | MGPWFM | 284 |
| GPLSGRSDNHRK | 285 | MIAPVAYR | 286 |
| GPLSGRSDNHRR | 287 | MLRSGW | 288 |
| GPPLGR | 289 | MLRSGWR | 290 |
| GPQGIAGQ | 291 | MLRSGWRG | 292 |
| GPQGLLGA | 293 | MLRSGWRL | 294 |
| GPRSFG | 295 | MLRSGWRS | 296 |
| GPRSFGL | 297 | MTFPTYIF | 298 |
| GPSHLVLT | 299 | MTFPTYWF | 300 |
| GPTN | 301 | NHRIGRSDNHRR | 302 |
| GPTNALAHGLF | 303 | NMPSFKLVTG | 304 |
| GRSML | 305 | NTLSGRSENHSG | 306 |
| GRSMLL | 307 | NTLSGRSGNHGS | 308 |
| GRSMLLG | 309 | NZPRVRLVLP | 310 |
| GRSMLLGG | 311 | PAGLWLDP | 312 |
| GRSMLLGP | 313 | PAGRR | 314 |
| GRSMLLGS | 315 | PAGRRS | 316 |
| GRSMLLP | 317 | PAGRRSL | 318 |
| GRSMLLPG | 319 | PASLWYTQ | 320 |
| GRSMLLPP | 321 | PESRRWMP | 322 |
| GRSMLLPS | 323 | PFHLSR | 324 |
| GRSMLLS | 325 | PHGFFQ | 326 |
| GRSMLLSG | 327 | PLARAGI | 328 |
| GRSMLLSP | 329 | PLARAGL | 330 |
| GRSMLLSS | 331 | PLGL | 332 |
| GRSMLM | 333 | PLGLAG | 17 |
| GRSMLMG | 334 | PLGLWA | 335 |
| GRSMLMGG | 336 | PLGVRGK | 337 |
| GRSMLMGP | 338 | PLTGRSGG | 339 |
| GRSMLMGS | 340 | PLTGRSGGGGSS | 341 |
| GRSMLMP | 342 | PPLGR | 343 |
| GRSMLMPG | 344 | PPPDMKLFPG | 345 |
| GRSMLMPP | 346 | PPPEVRSFSV | 347 |
| GRSMLMPS | 348 | PPPVLKLLEW | 349 |
| GRSMLMS | 350 | PPSIARSDNLAN | 351 |
| GRSMLMSG | 352 | PQHRIVSF | 353 |
| GRSMLMSP | 354 | PRFKIIGG | 355 |
| GRSMLMSS | 356 | PRFRIIGG | 357 |
| GSGPPLGR | 358 | PRPFVKSVDQ | 359 |
| GSPPLGR | 360 | PRQV | 361 |
| GSSPPLGR | 362 | PRSF | 363 |
| GTGRGPSWVGSS | 364 | PSPPVKMMPE | 365 |
| PTNL | 366 | PTNGGSGGSS | 367 |
| PTNLGSGGSS | 368 | RVPKVKVMLD | 369 |
| PVGYTSSL | 370 | SAGFSLPA | 371 |
| PVPRLKLIKD | 372 | SAPAVESE | 373 |
| PVQPIGPQ | 374 | SAPYFRMMDM | 375 |
| QALAMSAI | 376 | SARGPSRW | 377 |
| QFQALRM | 378 | SCGPPLGR | 379 |
| QGPMFKSLWD | 380 | SCSPPLGR | 381 |
| QGRAITFI | 382 | SGGPLGVR | 383 |
| QHQALRM | 384 | SGGPPLGR | 385 |
| QNQALRIA | 386 | SGPPLGR | 387 |
| QNQALRM | 388 | SGRS | 389 |
| QNQALRMA | 390 | SGRSA | 391 |
| QNQALRMAGGSGGSLSG | 392 | SGRSAN | 393 |
| RSDNH | | | |
| QNQALRMAGGSGGSLSG | 394 | SGRSANI | 395 |
| RSGNH | | | |
| QSRRVP | 396 | SGRSANP | 397 |
| QSRRVPL | 398 | SGRSANPRG | 399 |
| QSRRVPV | 400 | SGRSD | 401 |
| QTRRVP | 402 | SGRSDD | 403 |
| QTRRVPL | 404 | SGRSDDH | 405 |
| QTRRVPV | 406 | SGRSDI | 407 |
| QYIVSRSA | 408 | SGRSDIH | 409 |
| RALRAP | 410 | SGRSDN | 411 |
| REPFMKSLPW | 412 | SGRSDNI | 413 |
| RFPLKV | 414 | SGRSDNP | 415 |
| RFPSLKSFPL | 416 | SGRSDQ | 417 |
| RFPYGVW | 418 | SGRSDQH | 419 |
| RFYRNQFF | 420 | SGRSDT | 421 |
| RGPA | 422 | SGRSDTH | 423 |
| RGPAFNPM | 424 | SGRSDY | 425 |
| RGPATPIM | 426 | SGRSDYH | 427 |
| RGPKLYW | 428 | SGRSG | 429 |
| RHLAKL | 430 | SGRSGN | 431 |
| RIGRSDNH | 432 | SGRSGNH | 433 |
| RKMPNITV | 434 | SGSPPLGR | 435 |
| RKSSHIRMRDVVL | 436 | SIARSDNL | 437 |
| RKTVQHWW | 438 | SISSGLLSGRSDNI | 439 |
| RLGRSDNN | 440 | SNPFKY | 441 |
| RMHLRSLG | 442 | SPLPLRVP | 443 |
| RPLARAGI | 444 | SPLPLRVP | 445 |
| RPLARAGL | 446 | SPLTGRSG | 447 |
| RPLNGRSDNHKA | 448 | SPPLGR | 449 |
| RPLNGRSDNHKK | 450 | SRRVP | 451 |
| RPLNGRSDNHKR | 452 | SRRVPL | 453 |
| RPLNGRSDNHQA | 454 | SRRVPV | 455 |
| RPLNGRSDNHQK | 456 | SSGPPLGR | 457 |
| RPLNGRSDNHQR | 458 | SSPPLGR | 459 |
| RPLNGRSDNHRA | 460 | SSRGPAYL | 461 |
| RPLNGRSDNHRK | 462 | SSRHRRALD | 463 |
| RPLNGRSDNHRR | 464 | SSSFDKGKYKKGDDA | 465 |
| RPLSGRSDNHKA | 466 | SSSFDKGKYKRGDDA | 467 |
| RPLSGRSDNHKK | 468 | SSSPPLGR | 469 |
| RPLSGRSDNHKR | 470 | STFPFGMF | 471 |
| RPLSGRSDNHQA | 472 | STVFHM | 473 |
| RPLSGRSDNHQK | 474 | SVHHLI | 475 |
| RPLSGRSDNHQR | 476 | SVSGLLSH | 477 |
| RPLSGRSDNHRA | 478 | SVSGLLSS | 479 |
| RPLSGRSDNHRK | 480 | SVSGLRSH | 481 |
| RPLSGRSDNHRR | 482 | SVSGLRSS | 483 |
| RPSPMWAY | 484 | TARG | 485 |
| RRHDGLRA | 486 | TARGP | 487 |
| RRHDGLRS | 488 | TARGPALAHGLF | 489 |
| RSLVFAPI | 490 | TARGPS | 491 |
| RSPSRLKC | 492 | TARGPSF | 493 |
| VAGRSMRP | 494 | TARGPSFK | 495 |
| VAPQLKSLVP | 496 | TARGPSW | 497 |
| VAQFVLTE | 498 | TARGPSW | 499 |
| VHMPLGFLGP | 500 | TARGPVPRQV | 501 |
| VHMPLGFLGPGGLSGRSD | 502 | TFVH | 503 |
| NH | | | |
| VHMPLGFLGPGGTSTSGR | 504 | TGLSGRSVTQTS | 505 |
| SANPRG | | | |
| VLPELRSVFS | 506 | TGRGPSWV | 507 |
| VLSKQMSF | 508 | TLRLGRSDNNKN | 509 |
| VPAGRRS | 510 | TLSGLRSP | 511 |
| VPAGRRSL | 512 | TSGRSANP | 513 |
| VPRQ | 514 | TSGRSGNP | 515 |
| VPRQV | 516 | TSLSGRSANPRG | 517 |
| VSRSA | 518 | TSLSGRSGNPRG | 519 |
| VVPEGRRS | 520 | TSSGLRSP | 521 |
| WATPRPMR | 522 | TSTSGRSANPRG | 523 |
| WDHPISLL | 524 | TSTSGRSANPRGGGAVGL | 525 |
| | | LAPP | |
| QAR(A/V) | | TSTSGRSANPRGGGVHMP | 527 |
| | | LGFLGP | |
| YDPZVKVVLA | 528 | TSTSGRSGNPRG | 529 |
| YGAGLGVV | 530 | TVSGLRSP | 531 |
| YIVSRSA | 532 | | |
| YKKFVGSL | 533 | | |
| YVPRVKALEM | 534 | | |

In some embodiments, the CM may be or comprise a combination, a C-terminal truncation variant, or an N-terminal truncation variant of the example sequences discussed above. Truncation variants of the aforementioned amino acid sequences that are suitable for use in a CM may be any that retain the recognition site for the corresponding protease. These include C-terminal and/or N-terminal truncation variants comprising at least 3 contiguous amino acids of the above-described amino acid sequences, or at least 4, 5, 6, 7, 8, 9, or 10 amino acids of the foregoing amino acid sequences that retain a recognition site for a protease. In certain embodiments, the truncation variant of the above-described amino acid sequences may be an amino acid sequence corresponding to any of the above, but that is C- and/or N-terminally truncated by 1 to 10 amino acids, 1 to 9 amino acids, 1 to 8 amino acids, 1 to 7 amino acids, 1 to 6 amino acids, 1 to 5 amino acids, 1 to 4 amino acids, or 1 to 3 amino acids, and which: (1) has at least three amino acid residues; and (2) retains a recognition site for a protease. In some of the foregoing embodiments, the truncated CM is an N-terminally truncated CM. In some embodiments, the truncated CM is a C-terminally truncated CM. In some embodiments, the truncated C is a C- and an N-terminally truncated CM.

In some embodiments, the CM may comprise a total of 3 amino acids to 25 amino acids. In some embodiments, the CM may comprise a total of 3 to 25, 3 to 20, 3 to 15, 3 to 10, 3 to 5, 5 to 25, 5 to 20, 5 to 15, 5 to 10, 10 to 25, 10 to 20, 10 to 15, 15 to 25, 15 to 20, or 20 to 25 amino acids.

In some embodiments, the CM is specifically cleaved by at least a protease at a rate of about 0.001-1500×10⁴ M⁻¹ S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹ S⁻¹. The rate may be measured as substrate cleavage kinetics (k_{cat}/Kₘ) as disclosed in WO2016118629.

### Linkers (Ls)

The activatable TB (e.g., an activatable antibody or other therapeutic protein) may comprise one or more linkers (Ls). The linkers may comprise a stretch of amino acid sequence that link two components in the activatable TB. The linkers may be non-cleavable by any protease. In some embodiments, one or more linkers (e.g., flexible linkers) may be introduced into the activatable TB to provide flexibility at one or more of the junctions between domains, between moieties, between moieties and domains, or at any other junctions where a linker would be beneficial. In some embodiments, where the activatable TB is provided as a conformationally constrained construct, a flexible linker may be inserted to facilitate formation and maintenance of a structure in the uncleaved activatable TB. Any of the linkers described herein may provide the desired flexibility to facilitate the inhibition of the binding of a target, or to facilitate cleavage of a CM by a protease. In some embodiments, linkers included in the activatable TB may be all or partially flexible, such that the linker can include a flexible linker as well as one or more portions that confer less flexible structure to provide for a desired activatable TB. Some linkers may include cysteine residues, which may form non-alpha-carbon covalent bonds and reduce flexibility of the construct.

In most instances, linker length may be determined by counting, in a N- to C-direction, the number of amino acids from the N-terminus of the linker adjacent to the C-terminal amino acid of the preceding component, to the C-terminus of the linker adjacent to the N-terminal amino acid of the following component (i.e., where the linker length does not include either the C-terminal amino acid of the preceding component or the N-terminal amino acid of the following component).

In some embodiments, a linker may include a total of 1 to 50, 1 to 40, 1 to 30, 1 to 25 (e.g., 1 to 24, 1 to 22, 1 to 20, 1 to 18, 1 to 16, 1 to 15, 1 to 14, 1 to 12, 1 to 10, 1 to 8, 1 to 6, 1 to 5, 1 to 4, 1 to 3, 1 to 2, 2 to 25, 2 to 24, 2 to 22, 2 to 20, 2 to 18, 2 to 16, 2 to 15, 2 to 14, 2 to 12, 2 to 10, 2 to 8, 2 to 6, 2 to 5, 2 to 4, 2 to 3, 4 to 25, 4 to 24, 4 to 22, 4 to 20, 4 to 18, 4 to 16, 4 to 15, 4 to 14, 4 to 12, 4 to 10, 4 to 8, 4 to 6, 4 to 5, 5 to 25, 5 to 24, 5 to 22, 5 to 20, 5 to 18, 5 to 16, 5 to 15, 5 to 14, 5 to 12, 5 to 10, 5 to 8, 5 to 6, 6 to 25, 6 to 24, 6 to 22, 6 to 20, 6 to 18, 6 to 16, 6 to 15, 6 to 14, 6 to 12, 6 to 10, 6 to 8, 8 to 25, 8 to 24, 8 to 22, 8 to 20, 8 to 18, 8 to 16, 8 to 15, 8 to 14, 8 to 12, 8 to 10, 10 to 25, 10 to 24, 10 to 22, 10 to 20, 10 to 18, 10 to 16, 10 to 15, 10 to 14, 10 to 12, 12 to 25, 12 to 24, 12 to 22, 12 to 20, 12 to 18, 12 to 16, 12 to 15, 12 to 14, 14 to 25, 14 to 24, 14 to 22, 14 to 20, 14 to 18, 14 to 16, 14 to 15, 15 to 25, 15 to 24, 15 to 22, 15 to 20, 15 to 18, 15 to 16, 16 to 25, 16 to 24, 16 to 22, 16 to 20, 16 to 18, 18 to 25, 18 to 24, 18 to 22, 18 to 20, 20 to 25, 20 to 24, 20 to 22, 22 to 25, 22 to 24, or 24 to 25 amino acids). In some embodiments, the linker may include a total of 1, 2, 3, 4, 5, 6, 7, 8, 9, 10, 11, 12, 13, 14, 15, 16, 17, 18, 19, 20, 21, 22, 23, 24, or 25 amino acids.

In some embodiments, a linker may be rich in glycine (Gly or G) residues. In some embodiments, the linker may be rich in serine (Ser or S) residues. In some embodiments, the linker may be rich in glycine and serine residues. In some embodiments, the linker may have one or more glycine-serine residue pairs (GS) (e.g., 1, 2, 3, 4, 5, 6, 7, 8, 9, or 10 or more GS pairs).

In some embodiments, the linker may have one or more Gly-Gly-Gly-Ser (GGGS) (SEQ ID NO: 18) sequences (e.g., 1, 2, 3, 4, 5, 6, 7, 8, 9, or 10 or more GGGS sequences). In some embodiments, the linker may have one or more Gly-Gly-Gly-Gly-Ser (GGGGS) (SEQ ID NO: 535) sequences (e.g., 1, 2, 3, 4, 5, 6, 7, 8, 9, or 10 or more GGGGS sequences). In some embodiments, the linker may have one or more Gly-Gly-Ser-Gly (GGSG) (SEQ ID NO: 537) sequences (e.g., 1, 2, 3, 4, 5, 6, 7, 8, 9, or 10 or more GGSG (SEQ ID NO: 537) sequences). Examples of the linkers may include glycine polymers (G) n, glycine-serine polymers (including, for example, (GS) n, (GGS) n, (GSGGS) n (SEQ ID NO: 536) and (GGGS) n (SEQ ID NO: 18), where n is an integer of at least one), glycine-alanine polymers, alanine-serine polymers, and other flexible linkers known in the art. Glycine and glycine-serine polymers may be relatively unstructured, and therefore may be able to serve as a neutral link between components. Glycine accesses significantly more phi-psi space than even alanine, and is much less restricted than residues with longer side chains (see Scheraga, Rev. Computational Chem. 11173-142 (1992)). Example flexible linkers include one of or a combination of one or more of: GGSG (SEQ ID NO: 537), GGSGG (SEQ ID NO: 538), GSGSG (SEQ ID NO: 539), GSGGG (SEQ ID NO: 540), GGGSG (SEQ ID NO: 541), GSSSG (SEQ ID NO: 542), GSSGGSGGSGG (SEQ ID NO: 543), GGGS (SEQ ID NO: 18), GGGSGGGS (SEQ ID NO: 544), GGGSGGGSGGGS (SEQ ID NO: 545), GGGGSGGGGSGGGGS (SEQ ID NO: 546), GGGGSGGGGSGGGGSGGGGSGGGGS (SEQ ID NO: 547), GGGGSGGGGS (SEQ ID NO: 548), GGGGS (SEQ ID NO: 535), GS, GGGGSGS (SEQ ID NO: 549), GGGGSGGGGSGGGGSGS (SEQ ID NO: 550), GGSLDPKGGGGS (SEQ ID NO: 551), PKSCDKTHTCPPCPAPELLG (SEQ ID NO: 552), SKYGPPCPPCPAPEFLG (SEQ ID NO: 553), GKSSGSGSESKS (SEQ ID NO: 554), GSTSGSGKSSEGKG (SEQ ID NO: 555), GSTSGSGKSSEGSGSTKG (SEQ ID NO: 556), GSTSGSGKPGSGEGSTKG (SEQ ID NO: 557), GSTSGSGKPGSSEGST (SEQ ID NO: 558), GSTSGSGKPGSSEGST (SEQ ID NO: 559), GGGSSGGS (SEQ ID NO: 15), GGGGSGGGGSS (SEQ ID NO: 560), and GGGSSGGSGGSSGGS (SEQ ID NO: 561). Examples of linkers may further include a sequence that is at least 70% identical (e.g., at least 72%, at least 74%, at least 75%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, at least 99%, or 100% identical to the example linkers described herein. The ordinarily skilled artisan will recognize that design of activatable TBs can include linkers that are all or partially flexible, such that the linker can include a flexible linker as well as one or more portions that confer less flexible structure to provide for a desired activatable TB structure.

In some embodiments, an activatable TB may include one, two, three, four, five, six, seven, eight, nine, or ten linker sequence(s) (e.g., the same or different linker sequences of any of the exemplary linker sequences described herein or known in the art). In some embodiments, a linker may comprise sulfo-SIAB (sulfosuccinimidyl (4-iodoacetyl)aminobenzoate), SMPB (succinimidyl 4-(N-maleimidophenyl) butyrate), and sulfo-SMPB (sulfosuccinimidyl 4-(N-maleimidophenyl) butyrate), wherein the linkers react with primary amines sulfhydryls.

### Conjugation Agents

In some aspects, the activatable TBs (e.g., activatable ABs) may further comprise one or more additional agents, e.g., a targeting moiety to facilitate delivery to a cell or tissue of interest, a therapeutic agent (e.g., an antineoplastic agent such as chemotherapeutic or anti-neoplastic agent), a toxin, or a fragment thereof. The additional agents may be conjugated to the activatable TBs. The term “agent” is used herein to denote a chemical compound, a mixture of chemical compounds, a biological macromolecule, or an extract made from biological materials.

In some embodiments, the activatable TB may be conjugated to a cytotoxic agent, e.g., a toxin (e.g., an enzymatically active toxin of bacterial, fungal, plant, or animal origin, or fragments thereof) or a radioactive isotope.

Examples of cytotoxic agents that can be conjugated to the activatable TBs include: dolastatins and derivatives thereof (e.g., auristatin E, AFP, monomethyl auristatin D (MMAD), monomethyl auristatin F (MMAF), monomethyl auristatin E (MMAE), desmethyl auristatin E (DMAE), auristatin F, desmethyl auristatin F (DMAF), dolastatin 16 (DmJ), dolastatin 16 (Dpv), auristatin derivatives (e.g., auristatin tyramine, auristatin quinolone), maytansinoids (e.g., DM-1, DM-4), maytansinoid derivatives, duocarmycin, alpha-amanitin, turbostatin, phenstatin, hydroxyphenstatin, spongistatin 5, spongistatin 7, halistatin 1, halistatin 2, halistatin 3, halocomstatin, pyrrolobenzimidazoles (PBI), cibrostatin6, doxaliform, cemadotin analogue (CemCH2-SH), Pseudomonas toxin A (PES8) variant, Pseudomonase toxin A (ZZ-PE38) variant, ZJ-101, anthracycline, doxorubicin, daunorubicin, bryostatin, camptothecin, 7-substituted campothecin, 10, 11-difluoromethylenedioxycamptothecin, combretastatins, debromoaplysiatoxin, KahaMide-F, discodermolide, and Ecteinascidins.

Examples of enzymatically active toxins that can be conjugated to the activatable TBs include: diphtheria toxin, exotoxin A chain from Pseudomonas aeruginosa, ricin A chain, abrin A chain, modeccin A chain, alpha-sarcin, Aleuriies fordii proteins, dianfhin proteins, Phytoiaca Americana proteins (e.g., PAPI, PAPII, and PAP-8), Momordica charantia inhibitor, curcin, crotirs, Sapaonaria officinalis inhibitor, geionin, mitogeliin, restrictocin, phenomycin, neomycin, and tricothecenes.

Examples of anti-neoplastics that can be conjugated to the activatable TBs include: adriamycin, cerubidine, bleomycin, alkeran, velban, oncovin, fluorouracil, methotrexate, thiotepa, bisantrene, novantrone, thioguanine, procarabizine, and cytarabine.

Examples of antivirals that can be conjugated to the activatable TBs include: acyclovir, vira A, and symmetrel. Examples of antifungals that can be conjugated to the activatable TBs include: nystatin. Examples of detection reagents that can be conjugated to the activatable TBs include: fluorescein and derivatives thereof, fluorescein isothiocyanate (FITC). Examples of antibacterials that can be conjugated to the activatable TBs include: aminoglycosides, streptomycin, neomycin, kanamycin, amikacin, gentamicin, and tobramycin. Examples of 3beta, 16beta, 17alpha-trihydroxycholest-5-en-22-one 16-O)-(2-O-4-methoxybenzoyl-beta-D-xylopyranosyl)-(1->3)-(2-O-acetyl-alpha-L-arabinopyranoside) (OSW-1) that can be conjugated to the activatable TBs include: s-nitrobenzyloxycarbonyl derivatives of O6-benzylguanine, toposisomerase inhibitors, hemiasterlin, cephalotaxine, homoharringionine, pyrrol obenzodiazepine dimers (PBDs), functionalized pyrrolobenzodiazepenes, calcicheamicins, podophyiitoxins, taxanes, and vinca alkoids. Examples of radiopharmaceuticals that can be conjugated to the activatable TBs include: ¹²³I, ⁸⁹Zr, ¹²⁵I, ¹³¹I, ⁹⁹mTc, ²⁰¹Tl, ⁶²Cu, ¹⁸F, ⁶²Ga, ¹³N, ¹⁵O, ³⁸K, ⁸²Rb, ¹¹¹In, ¹³³Xe, ¹¹C, and ⁹⁹mTc (Technetium) Examples of heavy metals that can be conjugated to the activatable TBs include: barium, gold, and platinum. Examples of anti-mycoplasmals that can be conjugated to the activatable TBs include: tylosine, spectinomycin, streptomycin B, ampicillin, sulfanilamide, polymyxin, and chloramphenicol.

In some embodiments, the activatable TB may comprise a signal peptide. A signal peptide may be a peptide (e.g., 10-30 amino acids long) present at a terminus (e.g., the N-terminus or C-terminus) of a newly synthesized proteins that are destined toward the secretory pathway. In some embodiments, the signal peptide may be conjugated to the activatable TB via a spacer. In some embodiments, the spacer may be conjugated to the activatable TB in the absence of a signal peptide.

Those of ordinary skill in the art will recognize that a large variety of possible agents may be conjugated to any of the activatable TBs described herein. The agents may be conjugated to another component of the activatable TB by a conjugating moiety. Conjugation may include any chemical reaction that binds the two molecules so long as the activatable TB and the other moiety retain their respective activities. Conjugation may include many chemical mechanisms, e.g., covalent binding, affinity binding, intercalation, coordinate binding, and complexation. In some embodiments, the binding may be covalent binding. Covalent binding may be achieved either by direct condensation of existing side chains or by the incorporation of external bridging molecules. Many bivalent or polyvalent linking agents may be useful in conjugating any of the activatable TBs described herein. For example, conjugation may include organic compounds, such as thioesters, carbodiimides, succinimide esters, glutaraldehyde, diazobenzenes, and hexamethylene diamines. In some embodiments, the activatable TBs may include, or otherwise introduce, one or more non-natural amino acid residues to provide suitable sites for conjugation.

In some embodiments, an agent and/or conjugate may be attached by non-alpha-carbon covalent bonds (e.g., disulfide bonds on a cysteine molecule) to the antigen-binding domain. Since many cancers naturally release high levels of glutathione, a reducing agent, glutathione present in the cancerous tissue microenvironment can reduce the non-alpha-carbon covalent bonds, and subsequently release the agent and/or the conjugate at the site of delivery.

In some embodiments, when the conjugate binds to its target in the presence of complement within the target site (e.g., diseased tissue (e.g., cancerous tissue)), the amide or ester bond attaching the conjugate and/or agent to the linker is cleaved, resulting in the release of the conjugate and/or agent in its active form. These conjugates and/or agents when administered to a subject, may accomplish delivery and release of the conjugate and/or the agent at the target site (e.g., diseased tissue (e.g., cancerous tissue)). These conjugates and/or agents may be effective for the in vivo delivery of any of the conjugates and/or agents described herein.

In some embodiments, the conjugating moiety may be uncleavable by enzymes of the complement system. For example, the conjugate and/or agent is released without complement activation since complement activation ultimately lyses the target cell. In such embodiments, the conjugate and/or agent is to be delivered to the target cell (e.g., hormones, enzymes, corticosteroids, neurotransmitters, or genes). Furthermore, the conjugating moiety may be mildly susceptible to cleavage by serum proteases, and the conjugate and/or agent is released slowly at the target site.

In some embodiments, the conjugate and/or agent may be designed such that the conjugate and/or agent is delivered to the target site (e.g., disease tissue (e.g., cancerous tissue)) but the conjugate and/or agent is not released.

In some embodiments, the conjugate and/or agent may be attached to an antigen-binding domain either directly or via amino acids (e.g., D-amino acids), peptides, thiol-containing moieties, or other organic compounds that may be modified to include functional groups that can subsequently be utilized in attachment to antigen-binding domains by methods described herein.

In some embodiments, an activatable TB may include at least one point of conjugation for an agent. In some embodiments, all possible points of conjugation are available for conjugation to an agent. In some embodiments, the one or more points of conjugation may include sulfur atoms involved in non-alpha-carbon covalent bonds, sulfur atoms involved in interchain non-alpha-carbon covalent bonds, sulfur atoms involved in interchain sulfide bonds but not sulfur atoms involved in intrachain non-alpha-carbon covalent bonds, and/or sulfur atoms of cysteine or other amino acid residues containing a sulfur atom. In such cases, residues may occur naturally in the protein construct structure or may be incorporated into the protein construct using methods including site-directed mutagenesis, chemical conversion, or mis-incorporation of non-natural amino acids.

The present disclosure also provides methods and materials for preparing an activatable TB with one or more conjugated agents. In some embodiments, an activatable TB may be modified to include one or more interchain disulfide bonds. For example, disulfide bonds may undergo reduction following exposure to a reducing agent such as, without limitation, TCEP, DTT, or β-mercaptoethanol. In some cases, the reduction of the disulfide bonds may be only partial. As used herein, the term partial reduction refers to situations where an activatable TB is contacted with a reducing agent and a fraction of all possible sites of conjugation undergo reduction (e.g., not all disulfide bonds are reduced). In some embodiments, an activatable TB may be partially reduced following contact with a reducing agent if less than 99%, (e.g., less than 98%, 97%, 96%, 95%, 90%, 85%, 80%, 75%, 70%, 65%, 60%, 55%, 50%, 45%, 40%, 35%, 30%, 25%, 20%, 15%, 10% or 5%) of all possible sites of conjugation are reduced. In some embodiments, the activatable TB having a reduction in one or more interchain disulfide bonds may be conjugated to a drug reactive with free thiols.

The present disclosure also provides methods and materials for conjugating a therapeutic agent to a particular location on an activatable TB. In some embodiments, an activatable TB may be modified so that the therapeutic agents can be conjugated to the activatable TB at particular locations on the activatable TB. For example, an activatable TB may be partially reduced in a manner that facilitates conjugation to the activatable TB. In such cases, partial reduction of the activatable TB may occur in a manner that conjugation sites in the activatable TB are not reduced. In some embodiments, the conjugation site(s) on the activatable TB may be selected to facilitate conjugation of an agent at a particular location on the protein construct. Various factors can influence the “level of reduction” of the activatable TB upon treatment with a reducing agent. For example, without limitation, the ratio of reducing agent to activatable TB, length of incubation, incubation temperature, and/or pH of the reducing reaction solution can require optimization in order to achieve partial reduction of the activatable TB with the methods and materials described herein. Any appropriate combination of factors (e.g., ratio of reducing agent to activatable TB, the length and temperature of incubation with reducing agent, and/or pH of reducing agent) may be used to achieve partial reduction of the activatable TB (e.g., general reduction of possible conjugation sites or reduction at specific conjugation sites).

An effective ratio of reducing agent to activatable TB can be any ratio that at least partially reduces the activatable TB in a manner that allows conjugation to an agent (e.g., general reduction of possible conjugation sites or reduction at specific conjugation sites). In some embodiments, the ratio of reducing agent to activatable TB may be in a range from about 20:1 to 1:1, from 10:1 to 1:1, from 9:1 to 1:1, from 8:1 to 1:1, from 7:1 to 1:1, from 6:1 to 1:1, from 5:1 to 1:1, from 4:1 to 1:1, from 3:1 to 1:1, from 2:1 to 1:1, from 20:1 to 1:1.5, from 10.1 to 1:1.5, from 9:1 to 1:1.5, from 8:1 to 1:1.5, from 7:1 to 1:1.5, from 6:1 to 1:1.5, from 5:1 to 1:1.5, from 4:1 to 1:1.5, from 3:1 to 1:1.5, from 2:1 to 1.1.5, from 1.5:1 to 1:1.5, or from 1:1 to 1:1.5.

An effective incubation time and temperature for treating an activatable TB with a reducing agent may be any time and temperature that at least partially reduces the activatable TB in a manner that allows conjugation of an agent to an activatable TB (e.g., general reduction of possible conjugation sites or reduction at specific conjugation sites). In some embodiments, the incubation time and temperature for treating an activatable TB may be in a range from about 1 hour at 37° C. to about 12 hours at 37° C. (or any subranges therein).

An effective pH for a reduction reaction for treating an activatable TB with a reducing agent can be any pH that at least partially reduces the activatable TB in a manner that allows conjugation of the activatable TB to an agent (e.g., general reduction of possible conjugation sites or reduction at specific conjugation sites).

When a partially-reduced activatable TB is contacted with an agent containing thiols, the agent may conjugate to the interchain thiols in the activatable TB. An agent can be modified in a manner to include thiols using a thiol-containing reagent (e.g., cysteine or N-acetyl cysteine). For example, the activatable TB can be partially reduced following incubation with reducing agent (e.g., TEPC) for about 1 hour at about 37° C. at a desired ratio of reducing agent to activatable TB. An effective ratio of reducing agent to activatable TB may be any ratio that partially reduces at least two interchain disulfide bonds located in the activatable TB in a manner that allows conjugation of a thiol-containing agent (e.g., general reduction of possible conjugation sites or reduction at specific conjugation sites).

In some embodiments, an activatable TB may be reduced by a reducing agent in a manner that avoids reducing any intrachain disulfide bonds. In some embodiments of, an activatable TB may be reduced by a reducing agent in a manner that avoids reducing any intrachain disulfide bonds and reduces at least one interchain disulfide bond.

In some embodiments, the agent (e.g., agent conjugated to an activatable TB) may be a detectable moiety such as, for example, a label or other marker. For example, the agent may be or include a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties may be attached by spacer molecules. In some embodiments, the detectable label may include an imaging agent, a contrasting agent, an enzyme, a fluorescent label, a chromophore, a dye, one or more metal ions, or a ligand-based label. In some embodiments, the imaging agent may comprise a radioisotope. In some embodiments, the radioisotope may be indium or technetium. In some embodiments, the contrasting agent may comprise iodine, gadolinium or iron oxide. In some embodiments, the enzyme may comprise horseradish peroxidase, alkaline phosphatase, or β-galactosidase. In some embodiments, the fluorescent label may comprise yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments, the luminescent label may comprise an N-methylacrydium derivative. In some embodiments, the label may comprise an Alexa Fluor® label, such as Alex Fluor® 680 or Alexa Fluor® 750. In some embodiments, the ligand-based label may comprise biotin, avidin, streptavidin or one or more haptens.

In some embodiments, the agent may be conjugated to the activatable TB using a carbohydrate moiety, sulfhydryl group, amino group, or carboxylate group. In some embodiments, the agent may be conjugated to the activatable TB via a linker and/or a CM described herein. In some embodiments, the agent may be conjugated to a cysteine or a lysine in the activatable TB. In some embodiments, the agent may be conjugated to another residue of the activatable TB, such as those residues disclosed herein.

In some embodiments, a variety of bifunctional protein-coupling agents may be used to conjugate the agent to the activatable TB including N-succinimidyl-3-(2-pyridyldithiol) propionate (SPDP), iminothiolane (IT), bifunctional derivatives of imidoesters (e.g., dimethyl adipimidate HCL), active esters (e.g., disuccinimidyl suberate), aldehydes (e.g., glutareldehyde), bis-azido compounds (e.g., bis (p-azidobenzoyl) hexanediamine), bis-diazonium derivatives (e.g., bis-(p-diazoniumbenzoyl)-ethylenediamine), diisocyanates (e.g., tolyene 2,6-diisocyanate), and bis-active fluorine compounds (e.g., 1,5-difluoro-2,4-dinitrobenzene). For example, a ricin immunotoxin can be prepared as described in Vitetta et al., Science 238: 1098 (1987). In some embodiments, a carbon-14-labeled 1-isothiocyanatobenzyl-3-methyldiethylene triaminepentaacetic acid (MX-DTPA) chelating agent can be used to conjugate a radionucleotide to the activatable TB (See, e.g., WO94/11026).

Suitable conjugation moieties include those described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an activatable antibody by way of an oligopeptide. In some embodiments, suitable conjugation moieties include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #21651G), (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC. Additional example conjugation moieties include SMCC, sulfo-SMCC (sulfosuccinimidyl 4-(N-maleimidomethyl)cyclohexane-1-carboxylate), SPDB, and sulfo-SPDB.

The conjugation moieties described above may contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the SMPT contains a sterically-hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

Those of ordinary skill in the art will recognize that a large variety of possible moieties can be coupled to the activatable TB of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference). In general, an effective conjugation of an agent (e.g., cytotoxic agent) to an activatable TB can be accomplished by any chemical reaction that will bind the agent to the activatable TB while also allowing the agent and the activatable TB to retain functionality.

### Nucleic Acids and Vectors

In some aspects, the present disclosure further provides nucleic acids comprising sequences that encode the activatable target-binding protein (e.g., activatable antibody), or components or fragment thereof. The nucleic acids may comprise coding sequences for the TB, the CM, the MM, and the linker in an activatable TB. In cases where the activatable TB comprises multiple peptides, e.g., the activatable TBs comprise multiple peptides, the nucleic acid may comprise coding sequences for the multiple peptides. In some examples, the coding sequences for one of the peptides are comprised in a nucleic acid, and the coding sequences for another one of the peptides are comprised in another nucleic acid. In some examples, the coding sequences for two or more of the multiple peptides are comprised in the same nucleic acid.

Unless otherwise specified, a “nucleic acid sequence encoding a protein” includes all nucleotide sequences that are degenerate versions of each other and thus encode the same amino acid sequence. The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments, the nucleic acid is DNA. In some embodiments, the nucleic acid is RNA.

The term “N-terminally positioned” when referring to a position of a first domain or sequence relative to a second domain or sequence in a polypeptide primary amino acid sequence means that the first domain is located closer to the N-terminus of the polypeptide primary amino acid sequence. In some embodiments, there may be additional sequences and/or domains between the first domain or sequence and the second domain or sequence.

The term “C-terminally positioned” when referring to a position of a first domain or sequence relative to a second domain or sequence in a polypeptide primary amino acid sequence means that the first domain is located closer to the C-terminus of the polypeptide primary amino acid sequence. In some embodiments, there may be additional sequences and/or domains between the first domain or sequence and the second domain or sequence.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (e.g., asparagine and glutamine), alphatic family (e.g., alanine, valine, leucine and isoleucine), aromatic family (e.g., phenylalanine, tryptophan, and tyrosine).

The present disclosure further provides vectors and sets of vectors comprising any of the nucleic acids described herein. One skilled in the art will be capable of selecting suitable vectors or sets of vectors (e.g., expression vectors) for making any of the activatable TBs described herein, and using the vectors or sets of vectors to express any of the activatable TBs described herein. For example, in selecting a vector or a set of vectors, the type of cell may be selected such that the vector(s) may need to be able to integrate into a chromosome of the cell and/or replicate in it. Example vectors that can be used to produce an activatable TB are also described herein. As used herein, the term “vector” refers to a polynucleotide capable of inducing the expression of a recombinant protein (e.g., a first or second monomer) in a cell (e.g., any of the cells described herein). A “vector” is able to deliver nucleic acids and fragments thereof into a host cell, and includes regulatory sequences (e.g., promoter, enhancer, poly(A) signal). Exogenous polynucleotides may be inserted into the expression vector in order to be expressed. The term “vector” also includes artificial chromosomes, plasmids, retroviruses, and baculovirus vectors.

Methods for constructing suitable vectors that comprise any of the nucleic acids described herein, and suitable for transforming cells (e.g., mammalian cells) are well-known in the art. See, e.g., Sambrook et al., Eds. “Molecular Cloning: A Laboratory Manual,” 2^{nd} Ed., Cold Spring Harbor Press, 1989 and Ausubel et al., Eds. “Current Protocols in Molecular Biology,” Current Protocols, 1993.

Examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector may, for example, include sufficient cis-acting elements for expression; other elements for expression may be supplied by the host mammalian cell or in an in vitro expression system. Skilled practitioners will be capable of selecting suitable vectors and mammalian cells for making any activatable TB described herein.

In some embodiments, the activatable TB may be made biosynthetically using recombinant DNA technology and expression in eukaryotic or prokaryotic species.

### Cells

In some aspects, the present disclosure provides recombinant host cells comprising any of the vectors or nucleic acids described herein. The cells may be used to produce the activatable TBs (e.g., activatable antibodies) described herein. In some embodiments, the cell may be an animal cell, a mammalian cell (e.g., a human cell), a rodent cell (e.g., a mouse cell, a rat cell, a hamster cell, or a guinea pig cell), a non-human primate cell, an insect cell, a bacterial cell, a fungal cell, or a plant cell. In some embodiments, the cell may be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary (CHO) cells and human embryonic kidney cells (e.g., HEK293 cells). In some embodiments, the cell may be a prokaryotic cell.

Methods of introducing nucleic acids and vectors (e.g., any of the vectors or any of the sets of vectors described herein) into a cell are known in the art. Examples of methods that can be used to introducing a nucleic acid into a cell include: lipofection, transfection, calcium phosphate transfection, cationic polymer transfection, viral transduction (e.g., adenoviral transduction, lentiviral transduction), nanoparticle transfection, and electroporation.

In some embodiments, the introducing step includes introducing into a cell a vector (e.g., any of the vectors or sets of vectors described herein) including a nucleic acid encoding the monomers that make up any activatable TB described herein.

### Compositions and Kits

The present disclosure also provides compositions and kits comprising the activatable TBs described herein. The compositions and kits may further comprise one or more excipients, carriers, reagents, instructions needed for the use of the activatable TBs.

In some embodiments, the compositions may be pharmaceutical compositions, which comprise the activatable TBs, antibodies, derivatives, fragments, analogs and homologs thereof. The pharmaceutical compositions may comprise the activatable TB (e.g., antibody) and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition may be formulated to be compatible with its intended route of administration. Examples of routes of administration include parenteral, e.g., intravenous, intradermal, subcutaneous, oral (e.g., inhalation), transdermal (e.g., topical), transmucosal, and rectal administration. Solutions or suspensions used for parenteral, intradermal, or subcutaneous application may include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH may be adjusted with acids or bases, such as hydrochloric acid or sodium hydroxide. The parenteral preparation can be enclosed in ampoules, disposable syringes or multiple dose vials made of glass or plastic. In some, any of the activatable TBs described herein are prepared with carriers that protect against rapid elimination from the body, e.g., sustained and controlled release formulations, including implants and microencapsulated delivery systems. Biodegradable, biocompatible polymers can be used, e.g., ethylene vinyl acetate, polyanhydrides, polyglycolic acid, collagen, polyorthoesters, polylactic acid, and polylactic-co-glycolic acid. Methods for preparation of such pharmaceutical compositions and formulations are apparent to those skilled in the art.

In some embodiments, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition may be sterile and should be fluid and of a viscosity that facilitates easy syringeability. It may be stable under the conditions of manufacture and storage and preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. For dispersed particulate compositions, proper fluidity can be maintained, for example, by the use of a coating on the particles, such as lecithin, and by the maintenance of the required particle size in the case of dispersion and by the use of surfactants. In some embodiments, the pharmaceutical compositions may further comprise one or more antibacterial and/or antibacterial and antifungal agents, for example, parabens, chlorobutanol, phenol, ascorbic acid, thimerosal, and the like. In some embodiments, isotonic agents, for example, sugars, polyalcohols such as mannitol, sorbitol, and the like, as well as salts, such as, for example, sodium chloride, and the like may be included in the composition. Prolonged absorption of the injectable compositions may be brought about by including in the composition an agent that delays absorption, for example, aluminum monostearate and gelatin.

In some embodiments, the pharmaceutical composition may comprise a sterile injectable solution. Sterile injectable solutions may be prepared by incorporating the active compound in the required amount in an appropriate solvent with one or a combination of ingredients enumerated above, as required, followed by filtered sterilization. Generally, dispersions may be prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, the pharmaceutical composition may comprise an oral composition. Oral compositions may include an inert diluent or an edible carrier. They may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, the pharmaceutical composition may be formulized for administration by inhalation. For example, the compounds may be delivered in the form of an aerosol spray from pressured container or dispenser that contains a suitable propellant, e.g., a gas such as carbon dioxide, or a nebulizer.

In some embodiments, the pharmaceutical composition may be formulized for systemic administration. For example, systemic administration may be by intravenous, as well by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds may be formulated into ointments, salves, gels, or creams as generally known in the art.

In some embodiments, the pharmaceutical composition may be prepared in the form of suppositories (e.g., with conventional suppository bases such as cocoa butter and other glycerides) or retention enemas for rectal delivery.

In one embodiment, the pharmaceutical composition may be prepared with carriers that protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid, and polylactic-co-glycolic acid. Methods for preparation of such formulations will be apparent to those skilled in the art.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure may be dictated by and directly dependent on the unique characteristics of the active compound and the particular therapeutic effect to be achieved, and the limitations inherent in the art of compounding such an active compound for the treatment of individuals.

In some embodiments, the compositions (e.g., pharmaceutical compositions) may be included in a container, vial, syringe, injector pen, pack, or dispenser, optionally together with instructions for administration.

Also provided herein are kits that include any of the activatable TBs described herein, any of the compositions that include any of the activatable TBs described herein, or any of the pharmaceutical compositions that include any of the activatable TBs described herein. Also provided are kits that include one or more second therapeutic agent(s) in addition to an activatable TB described herein. The second therapeutic agent(s) may be provided in a dosage administration form that is separate from the activatable TBs. Alternatively, the second therapeutic agent(s) may be formulated together with the activatable TBs.

Any of the kits described herein can include instructions for using any of the compositions (e.g., pharmaceutical compositions) and/or any of the activatable TBs described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

Also provided herein are activatable TBs produced by any of the methods described herein. Also provided are compositions (e.g., pharmaceutical compositions) that comprise any of the activatable TBs produced by any of the methods described herein. Also provided herein are kits that include at least one dose of any of the compositions (e.g., pharmaceutical compositions) described herein.

### Methods of Producing Activatable Target-Binding Proteins

Provided herein are methods of producing any activatable TB described herein that include: (a) culturing any of the recombinant host cells described herein in a liquid culture medium under conditions sufficient to produce the activatable TB; and (b) recovering the activatable TB from the host cell and/or the liquid culture medium.

Methods of culturing cells are well known in the art. In some embodiments, cells may be maintained in vitro under conditions that favor cell proliferation, cell differentiation and cell growth. For example, the recombinant cells may be cultured by contacting a cell (e.g., any of the cells described herein) with a cell culture medium that includes the necessary growth factors and supplements sufficient to support cell viability and growth.

In some embodiments, the method may further include isolating the recovered activatable TB. The isolation of the activatable TB may be performed using any separation or purification technique for separating protein species, e.g., affinity tag-based protein purification (e.g., polyhistidine (His) tag, glutathione-S-transferase tag, and the like), ammonium sulfate precipitation, polyethylene glycol precipitation, size exclusion chromatography, ligand-affinity chromatography (e.g., Protein A chromatography), ion-exchange chromatography (e.g., anion or cation), hydrophobic interaction chromatography, and the like.

Compositions and methods described herein may involve use of non-reducing or partially-reducing conditions that allow non-alpha-carbon covalent bonds, e.g., disulfide bonds to form between the MM and the AB of the activatable TBs.

In some embodiments, a dual-anchored activatable macromolecule (e.g., dual-anchored activatable TB or dual-anchored activatable antibody) of the present disclosure is prepared by a method comprising: engineering a cysteine residue at a disulfide bonding site in a MM of the dual-anchored activatable macromolecule; engineering a cysteine residue at a disulfide bonding site in a TB of the dual-anchored activatable macromolecule, wherein the MM and the TB are coupled and a CM is positioned between the MM and the TB. In some aspects, the present disclosure includes expressing an activatable macromolecule having an engineered cysteine residue at a disulfide bonding site in a TB of the dual-anchored activatable macromolecule and recovering the dual-anchored activatable macromolecule, wherein the MM and the TB are tethered at their disulfide bonding sites in the recovered dual-anchored activatable macromolecule.

In some embodiments, a dual-anchored activatable macromolecule (e.g., dual-anchored activatable TB or dual-anchored activatable antibody) of the present disclosure is prepared by a method comprising coupling a MM comprising a cysteine to a TB, wherein the cysteine forms a non-alpha-carbon covalent bond with a non-alpha-carbon covalent bond-forming amino acid in the TB.

In some embodiments, a dual-anchored activatable macromolecule (e.g., dual-anchored activatable TB or dual-anchored activatable antibody) of the present disclosure is prepared by a method comprising coupling a TB to a MM comprising a non-alpha-carbon covalent bond-forming amino acid, wherein the MM forms a non-alpha-carbon covalent bond with a non-alpha-carbon covalent bond-forming amino acid in the TB.

In some aspects, the present disclosure includes a method of making a dual-anchored activatable macromolecule comprising providing a MM comprising a non-alpha-carbon covalent bond-forming amino acid configured to form a non-alpha-carbon covalent bond with a non-alpha-carbon covalent bond-forming amino acid in a TB that is coupled to the MM. In some aspects, the present disclosure includes a method of making a dual-anchored activatable macromolecule comprising providing a MM comprising a cysteine configured to form a non-alpha-carbon covalent bond with a non-alpha-carbon covalent bond-forming amino acid in a TB that is coupled to the MM.

In some aspects, the present disclosure includes a method of identifying a position for inserting a non-alpha-carbon covalent bond-forming amino acid in a dual-anchored activatable macromolecule. In some aspects, the method includes analyzing the structural arrangement of a TB and a MM in the dual-anchored activatable macromolecule and identifying a position within the TB for inserting the non-alpha-carbon covalent bond-forming amino acid. In some aspects, the method includes analyzing the structural arrangement of a TB and a MM in the dual-anchored activatable macromolecule and identifying a position within the MM for inserting the non-alpha-carbon covalent bond-forming amino acid. In some aspects, the method includes analyzing the structural arrangement of a TB and a MM in the dual-anchored activatable macromolecule and identifying a position within a peptide coupled to the TB for inserting the non-alpha-carbon covalent bond-forming amino acid. In some aspects, the method includes analyzing the structural arrangement of a TB and a MM in the dual-anchored activatable macromolecule and identifying a position within a peptide coupled to the MM for inserting the non-alpha-carbon covalent bond-forming amino acid. In some aspects, one or more of the non-alpha-carbon covalent bond-forming amino acids is a cysteine.

In some aspects, the present disclosure includes a method of identifying a position for inserting a non-alpha-carbon covalent bond-forming amino acid in a dual-anchored activatable macromolecule comprising mapping a three-dimensional structure of a TB coupled to a MM, identifying a first amino acid of the TB located proximal to a second amino acid of the MM, wherein the first amino acid is a position for inserting a non-alpha-carbon covalent bond-forming amino acid in the TB. In some aspects, the second amino acid is a position for inserting a non-alpha-carbon covalent bond-forming amino acid in the MM. In some aspects, the alpha Carbon atom of the first amino acid is located within 2 to 15 angstroms of the alpha Carbon atom of the second amino acid in the three-dimensional structure of the TB coupled to the MM. In some aspects, the alpha Carbon atom of the first amino acid is located within 15, 10, 9, 8, 7, 6, 5, 4, 3, or 2 angstroms of the alpha Carbon atom of the second amino acid in the three-dimensional structure of the TB coupled to the MM. In cases where one or both of the amino acids are in flexible loops, the above inter-Cox distances apply to the case when the two amino acids are closest to one another. In some aspects, the non-alpha-carbon covalent bonds include, but are not limited to:
1. Disulfide bonds between two Cys residues.
2. Isopeptide bonds between Lys and Glutamate or Glutamine residues.
3. Ester bonds between Thr and Glutamine.
4. Thioester bonds between Cys and Gln.
5. Thioether bonds between Cys and Tyr.
6. His-Tyr crosslinks between His and Tyr (e.g., as known to exist in cytochrome c oxidase type proteins).
7. Lys-Cys NOS (Nitrogen-Oxygen-Sulfur) crosslinks.

In some aspects, the present disclosure includes a method of making a dual-anchored activatable macromolecule comprising coupling a MM comprising a cysteine to a TB, wherein the cysteine forms a non-alpha-carbon covalent bond with a non-alpha-carbon covalent bond-forming amino acid in the TB.

In some aspects, the present disclosure includes a method of making a dual-anchored activatable macromolecule comprising coupling a TB to a MM comprising a non-alpha-carbon covalent bond-forming amino acid, wherein the MM forms a non-alpha-carbon covalent bond with a non-alpha-carbon covalent bond-forming amino acid in the TB.

In some embodiments, the method further includes formulating the isolated activatable TB into a pharmaceutical composition. Various formulations are known in the art and are described herein. Any isolated activatable TB described herein can be formulated for any route of administration (e.g., intravenous, intratumoral, subcutaneous, intradermal, oral, inhalation, intranasal, intrapulmonary, intrathecal, infusion, transdermal, topical, transmucosal, or intramuscular).

### Methods of Treatment

In some aspects, the present disclosure further provides methods of treating a disease (e.g., a cancer (e.g., any of the cancers described herein), an inflammatory condition, disorder or disease, or an autoimmune condition, disorder or disease) in a subject including administering a therapeutically effective amount of any of the activatable TBs described herein to the subject. In some embodiments, the disclosure provides methods of preventing, delaying the progression of, treating, alleviating a symptom of, or otherwise ameliorating disease in a subject by administering a therapeutically effective amount of an activatable TB described herein to a subject in need thereof. The term “treatment” refers to ameliorating at least one symptom of a disorder. In some embodiments, the disorder being treated is a cancer and to ameliorate at least one symptom of a cancer. As used herein, the term “subject” refers to any mammal. In some embodiments, the subject is a feline (e.g., a cat), a canine (e.g., a dog), an equine (e.g., a horse), a rabbit, a pig, a rodent (e.g., a mouse, a rat, a hamster or a guinea pig), a non-human primate (e.g., a simian (e.g., a monkey (e.g., a baboon, a marmoset), or an ape (e.g., a chimpanzee, a gorilla, an orangutan, or a gibbon)), or a human. In some embodiments, the subject is a human. The terms subject and patient are used interchangeably herein. In some embodiments, the subject has been previously identified or diagnosed as having the disease (e.g., cancer (e.g., any of the cancers described herein)).

The activatable TB used in any of the embodiments of these methods and uses may be administered at any stage of the disease. For example, such an activatable TB may be administered to a patient suffering cancer of any stage, from early to metastatic. In some embodiments, the activatable TB and formulations thereof may be administered to a subject suffering from or susceptible to a disease or disorder associated with aberrant target expression and/or activity.

A subject suffering from or susceptible to a disease or disorder associated with aberrant target expression and/or activity may be identified using any of a variety of methods known in the art. For example, subjects suffering from an inflammatory condition, disorder or disease, or an autoimmune condition, disorder or disease, cancer or other neoplastic condition may be identified using any of a variety of clinical and/or laboratory tests such as, physical examination and blood, urine and/or stool analysis to evaluate health status. For example, subjects suffering from inflammation and/or an inflammatory disorder may be identified using any of a variety of clinical and/or laboratory tests such as physical examination and/or bodily fluid analysis, e.g., blood, urine and/or stool analysis, to evaluate health status.

In some embodiments, administration of an activatable TB to a patient suffering from a disease or disorder associated with aberrant target expression and/or activity may be considered successful if any of a variety of laboratory or clinical objectives is achieved. For example, administration of an activatable TB to a patient suffering from a disease or disorder associated with aberrant target expression and/or activity may be considered successful if one or more of the symptoms associated with the disease or disorder is alleviated, reduced, inhibited or does not progress to a further, i.e., worse, state. Administration of an activatable TB to a patient suffering from a disease or disorder associated with aberrant target expression and/or activity may be considered successful if the disease or disorder enters remission or does not progress to a further, i.e., worse, state.

As used herein, the term “treat” includes reducing the severity, frequency or the number of one or more (e.g., 1, 2, 3, 4, or 5) symptoms or signs of a disease (e.g., a cancer (e.g., any of the cancers described herein)) in the subject (e.g., any of the subjects described herein). In some embodiments where the disease is cancer, treating results in reducing cancer growth, inhibiting cancer progression, inhibiting cancer metastasis, or reducing the risk of cancer recurrence in a subject having cancer.

In some embodiments, the disease may be a cancer. In some embodiments, the subject may have been identified or diagnosed as having a cancer. Examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, a lymphoma (e.g., B-cell lymphoma, B-cell non-Hodgkin's lymphoma, Hodgkin's lymphoma, cutaneous T-cell lymphoma), a leukemia (e.g., hairy cell leukemia, chronic lymphocytic leukemia (CLL), acute myeloid leukemia (AML), chronic myeloid leukemia (CML), acute lymphocytic leukemia (ALL)), myelodysplastic syndromes (MDS), Kaposi sarcoma, retinoblastoma, stomach cancer, urothelial carcinoma, lung cancer, renal cell carcinoma, gastric and esophageal cancer, pancreatic cancer, prostate cancer, brain cancer, colon cancer, bone cancer, lung cancer, breast cancer, colorectal cancer, ovarian cancer, nasopharyngeal adenocarcinoma, non-small cell lung carcinoma (NSCLC), squamous cell head and neck carcinoma, endometrial cancer, bladder cancer, cervical cancer, liver cancer, and hepatocellular carcinoma. In some embodiments, the cancer is a lymphoma. In some embodiments, the lymphoma is Burkitt's lymphoma. In some aspects, the subject has been identified or diagnosed as having familial cancer syndromes such as Li Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. Further examples of cancers that may be treated by the compositions and methods herein include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma, AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer, Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous, Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell, Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer, Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer, Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood, Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer, Uterine Sarcoma; Vaginal Cancer; Visual Pathway and Hypothalamic Glioma, Childhood; Vulvar Cancer; Waldenstrom's Macro globulinemia; Wilms' Tumor; diffuse large B-cell lymphoma (DLBCL); and mantle cell lymphoma (MCL). Metastases of the aforementioned cancers may also be treated or prevented in accordance with the methods described herein.

In some embodiments, the methods herein may result in a reduction in the number, severity, or frequency of one or more symptoms of the cancer in the subject (e.g., as compared to the number, severity, or frequency of the one or more symptoms of the cancer in the subject prior to treatment).

The methods may further comprise administering to a subject one or more additional agents. In some embodiments, the activatable TB may be administered during and/or after treatment in combination with one or more additional agents. In some embodiments, the activatable TB may be formulated into a single therapeutic composition, and the activatable TB and additional agent(s) may be administered simultaneously. Alternatively, the activatable TB and additional agent(s) may be separate from each other, e.g., each is formulated into a separate therapeutic composition, and the activatable TB and the additional agent are administered simultaneously, or the activatable TB and the additional agent are administered at different times during a treatment regimen. For example, the activatable TB may be administered prior to the administration of the additional agent, subsequent to the administration of the additional agent, or in an alternating fashion. The activatable TB and additional agent(s) may be administered in single doses or in multiple doses.

The present disclosure also provides methods of detecting presence or absence of a cleaving agent and the target in a subject or a sample. Such methods may comprise (i) contacting a subject or biological sample with an activatable TB, wherein the activatable TB includes a detectable label that is positioned on a portion of the activatable TB that is released following cleavage of the CM and (ii) measuring a level of activated (cleaved) TB in the subject or biological sample, wherein a detectable level of activated TB in the subject or biological sample indicates that the cleaving agent, the target or both the cleaving agent and the target are absent and/or not sufficiently present in the subject or biological sample, such that the target binding and/or protease cleavage of the activatable TB cannot be detected in the subject or biological sample, and wherein a reduced detectable level of activated (cleaved) TB in the subject or biological sample indicates that the cleaving agent and the target are present in the subject or biological sample.

A reduced level of detectable label may be, for example, a reduction of at least 5%, 10%, 15%, 20%, 25%, 30%, 35%, 40%, 45%, 50%, 55%, 60%, 65%, 70%, 75%, 80%, 85%, 90%, or 95%, or a reduction of substantially 100%. In some embodiments, the detectable label may be conjugated to a component of the activatable TB, e.g., the AB. In some embodiments, measuring the level of activatable TB in the subject or sample may be accomplished using a secondary reagent that specifically binds to the activated TB, wherein the reagent comprises a detectable label. The secondary reagent may be a TB comprising a detectable label.

In some embodiments, the activatable TBs may also be useful in the detection of the target in patient samples and accordingly are useful as diagnostics. For example, the activatable TBs may be used in in vitro assays, e.g., ELISA, to detect target levels in a patient sample. For example, an activatable TB may be immobilized on a solid support (e.g., the well(s) of a microtiter plate). The immobilized activatable TB may serve as a capture TB for any target that may be present in a test sample. Prior to contacting the immobilized TB with a patient sample, the solid support may be rinsed and treated with a blocking agent such as milk protein or albumin to prevent nonspecific adsorption of the analyte.

In some embodiments, based on the results obtained using the activatable TBs in an in vitro diagnostic assay, the stage of a disease in a subject may be determined based on expression levels of the target antigen(s). For a given disease, samples of blood may be taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

Activatable TBs herein may also be used in diagnostic and/or imaging methods. In some embodiments, such methods may be in vitro methods. In some embodiments, such methods may be in vivo methods. In some embodiments, such methods may be in situ methods. In some embodiments, such methods may be ex vivo methods. For example, activatable TBs having a CM may be used to detect the presence or absence of an enzyme capable of cleaving the CM. Such activatable TBs may be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated TBs (i.e., TBs resulting from cleavage of an activatable TB) in a given cell or tissue of a given host organism. Such accumulation of activated TBs indicates not only that the tissue expresses enzymatic activity (or an increased reduction potential depending on the nature of the CM) but also that the tissue expresses target to which the activated TB binds.

For example, the CM may be selected to be a protease substrate for a protease found at the site of a tumor, at the site of a viral or bacterial infection at a biologically confined site (e.g., such as in an abscess, in an organ, and the like), and the like. The AB may be one that binds a target antigen. Using methods familiar to one skilled in the art, a detectable label (e.g., a fluorescent label or radioactive label or radiotracer) may be conjugated to an AB or other region of an activatable TB. Suitable detectable labels may be discussed in the context of the above screening methods and additional specific examples are provided below. Using a TB (e.g., an AB) specific to a protein or peptide of the disease state, along with a protease whose activity is elevated in the disease tissue of interest, activatable TBs may exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated TBs in the disease tissue may be enhanced relative to non-disease tissues.

In some embodiments, the activatable TBs may be useful for in vivo imaging where detection of the fluorescent signal in a subject, e.g., a mammal, including a human, indicates that the disease site contains the target and contains a protease that is specific for the CM of the activatable TB. The in vivo imaging may be used to identify or otherwise refine a patient population suitable for treatment with an activatable TB of the disclosure. For example, patients that test positive for both the target and a protease that cleaves the substrate in the CM of the activatable TB being tested (e.g., accumulate activated TBs at the disease site) are identified as suitable candidates for treatment with such an activatable TB comprising such a CM. Likewise, patients that test negative may be identified as suitable candidates for another form of therapy (i.e., not suitable for treatment with the activatable TB being tested). In some embodiments, such patients that test negative with respect to a first activatable TB can be tested with other activatable TBs comprising different CMs until a suitable activatable TB for treatment is identified (e.g., an activatable TB comprising a CM that is cleaved by the patient at the site of disease).

In some embodiments, in situ imaging may be useful in methods to identify which patients to treat. For example, in in situ imaging, the activatable TBs may be used to screen patient samples to identify those patients having the appropriate protease(s) and target(s) at the appropriate location, e.g., at a tumor site. In some embodiments, in situ imaging is used to identify or otherwise refine a patient population suitable for treatment with an activatable TB of the disclosure. For example, patients that test positive for both the target and a protease that cleaves the substrate in the CM of the activatable TB being tested (e.g., accumulate activated TBs at the disease site) are identified as suitable candidates for treatment with such an activatable TB comprising such a CM. Likewise, patients that test negative for either or both of the target and the protease that cleaves the substrate in the CM used in the activatable TB being tested using these methods are identified as suitable candidates for another form of therapy (i.e., not suitable for treatment with the activatable TB being tested). In some embodiments, such patients that test negative with respect to a first activatable TB can be tested with other activatable TBs comprising different CMs until a suitable activatable TB for treatment is identified (e.g., an activatable TB comprising a CM that is cleaved by the patient at the site of disease).

In some embodiments, the present disclosure includes any one or combination of the following non-limiting numbered items:
1. A dual-anchored activatable target-binding protein comprising:
a target-binding protein (TB) that specifically binds to a target;
a masking moiety (MM) coupled to the TB, wherein the MM inhibits binding of the AB to the target; and
a cleavable moiety (CM) coupled to the TB and positioned between the TB and the MM, wherein the CM is a polypeptide that functions as a substrate for a protease, and
further comprising a non-alpha-carbon covalent bond tethering the MM and the TB.
2. The activatable target-binding protein of item 1, wherein the TB is an antigen-binding protein (AB).
3. The activatable target-binding protein of item 1 or item 2, wherein the activatable target-binding protein has a lower target-binding activity compared to a single-anchored activatable target-binding protein lacking the non-alpha-carbon covalent bond.
4. The activatable target-binding protein of any one or combination of items 1-3, wherein the non-alpha-carbon covalent bond is an ester bond or a thioester bond.
5. The activatable target-binding protein of item 4, wherein the ester bond is between a threonine and a glutamine.
6. The activatable target-binding protein of item 4, wherein the thioester bond is between a cysteine and a glutamine or a tyrosine.
7. The activatable target-binding protein of any one or combination of items 1-3, wherein the non-alpha-carbon covalent bond is a cross-link between a histidine and a tyrosine or a cross-link between a lysine and a cysteine.
8. The activatable target-binding protein of any one or combination of items 1-3, wherein the non-alpha-carbon covalent bond is an isopeptide bond.
9. The activatable target-binding protein of item 8, wherein the isopeptide bond is between a lysine and a glutamate or aspartate residue.
10. The activatable target-binding protein of item 8, wherein the isopeptide bond is between a gamma-carboxyamide group of glutamine and epsilon-amino group of a lysine sidechain.
11. The activatable target-binding protein of any one or combination of items 1-3, wherein the non-alpha-carbon covalent bond is a disulfide bond.
12. The activatable target-binding protein of item 11, wherein the disulfide bond is formed between a first cysteine and a second cysteine, wherein
the first cysteine is within the MM and the second cysteine is within the TB,
the first cysteine is within a peptide coupled to the MM and the second cysteine is within the TB, or
the first cysteine is within the MM and the second cysteine is within a peptide coupled to the TB.
13. The activatable target-binding protein of any one or combination of items 1-12, further comprising a second CM, wherein
the second CM is positioned between the MM and the non-alpha-carbon covalent bond,
the second CM is within the MM and up to 5 amino acids away from a cysteine forming the non-alpha-carbon covalent bond, or
the second CM is within the TB and at up to 5 amino acids away from a cysteine forming the non-alpha-carbon covalent bond.
14. The activatable target-binding protein of any one or combination of items 1-13, wherein the first and the second CMs are substrates of different proteases.
15. The activatable target-binding protein of any one or combination of items 1-13, wherein the first and the second CMs are substrates of the same protease.
16. The activatable target-binding protein of any one or combination of items 1-12, wherein the protease is produced by a tumor in a subject.
17. The activatable target-binding protein of any one or combination of items 2-16, wherein the AB is an antibody, a Fab fragment, a F(ab′)₂ fragment, a scFv, a scAb, a dAb, a VHH, or a single domain antibody.
18. The activatable target-binding protein of any one or combination of items 2-17, wherein the AB is a single domain antibody.
19. The activatable target-binding protein of any one or combination of items 2-18, wherein the AB is an Fc-tagged single domain antibody.
20. The activatable target-binding protein of any one or combination of items 2-17, wherein the AB is a bispecific antibody.
21. The activatable target-binding protein of item 20, wherein the bispecific antibody is a bispecific T Cell engager (BiTE) or a dual-affinity retargeting antibody (DART).
22. The activatable target-binding protein of any one or combination of items 2-17, wherein the AB is a multispecific antibody.
23. The activatable target-binding protein of any one or combination of items 18-19, wherein the non-alpha-carbon covalent bond is between the MM and the single domain antibody.
24. The activatable target-binding protein of any one or combination of items 1-22, wherein the non-alpha-carbon covalent bond is between the MM and an Fc domain.
25. The activatable target-binding protein of any one or combination of items 1-24, wherein the MM comprises an epitope of the TB.
26. The activatable target-binding protein of any one or combination of items 1-24, wherein the MM does not comprise four or more consecutive amino acids of an epitope of the TB.
27. The activatable target-binding protein of any one or combination of items 1-26, wherein the MM has a dissociation constant for binding to the TB that is greater than a dissociation constant of the TB for binding to the target.
28. The activatable target-binding protein of any one or combination of items 1-27, wherein the MM is a polypeptide of from 2 to 40 amino acids in length.
29. The activatable target-binding protein of any one or combination of items 1-28, wherein the activatable target-binding protein comprises a linker between the MM and the CM.
30. The activatable target-binding protein of any one or combination of items 1-29, wherein the activatable target-binding protein comprises a linker between CM and the TB.
31. The activatable target-binding protein of any one or combination of items 1-29, wherein the activatable target-binding protein comprises a first linker between the MM and the CM and a second linker between the CM and the TB.
32. A composition comprising the activatable target-binding protein of any one or combination of items 1 to 31 and a carrier.
33. The composition of item 32, wherein the composition is a pharmaceutical composition.
34. A container, vial, syringe, injector pen, or kit comprising at least one dose of the composition of item 32 or 33.
35. A nucleic acid comprising a sequence encoding the activatable target-binding protein of any one or combination of items 1 to 31.
36. A vector comprising the nucleic acid of item 35.
37. A cell comprising the nucleic acid of item 35 or the vector of item 36.
38. A conjugated activatable target-binding protein comprising the activatable target-binding protein of any one or combination of items 1 to 31 conjugated to an agent.
39. The conjugated activatable target-binding protein of item 38, wherein the agent is a therapeutic agent, a targeting moiety, or a detectable moiety.
40. A method of treating a subject in need thereof comprising administering to the subject a therapeutically effective amount of the activatable target-binding protein of any one or combination of items 1 to 31, the composition of item 32 or 33, or the conjugated activatable target-binding protein of item 38 or 39.
41. The method of item 40, wherein the subject has been identified or diagnosed as having a cancer, an inflammatory condition, disorder or disease, or an autoimmune condition, disorder or disease.
42. A method of producing an activatable target-binding protein, comprising:
culturing the cell of item 37 in a culture medium under a condition sufficient to produce the activatable target-binding protein; and
recovering the activatable target-binding protein from the cell or the culture medium.
43. The method of item 42, further comprising isolating the activatable target-binding protein recovered from the cell or the culture medium.
44. The method of item 43, wherein the isolating is performed using a protein purification tag and/or size exclusion chromatography.
45. The method of item 43 or item 44, further comprising formulating the activatable target-binding protein into a pharmaceutical composition.
46. A method of producing a dual-anchored activatable macromolecule comprising: engineering a cysteine residue at a disulfide bonding site in a masking moiety (MM) of the dual-anchored activatable macromolecule; engineering a cysteine residue at a disulfide bonding site in a target-binding protein (TB) of the dual-anchored activatable macromolecule, wherein the MM and the TB are coupled and a cleavable moiety (CM) is positioned between the MM and the TB, expressing the dual-anchored activatable macromolecule; and recovering the dual-anchored activatable macromolecule, wherein the MM and the TB are tethered at their disulfide bonding sites in the recovered dual-anchored activatable macromolecule.
47. A method of producing a dual-anchored activatable macromolecule comprising: engineering an arginine or lysine residue at an isopeptide bonding site in a masking moiety (MM) of the dual-anchored activatable macromolecule and/or engineering an aspartate or glutamate residue at an isopeptide bonding site in a target-binding protein (TB) of the dual-anchored activatable macromolecule, wherein the MM and the TB are coupled and a cleavable moiety (CM) is positioned between the MM and the TB, expressing the dual-anchored activatable macromolecule; and recovering the dual-anchored activatable macromolecule, wherein the MM and the TB are tethered at their isopeptide bonding sites in the recovered dual-anchored activatable macromolecule.
48. A method of producing a dual-anchored activatable macromolecule comprising: engineering an aspartate or glutamate residue at an isopeptide bonding site in a masking moiety (MM) of the dual-anchored activatable macromolecule and/or engineering an arginine or lysine residue at an isopeptide bonding site in a target-binding protein (TB) of the dual-anchored activatable macromolecule, wherein the MM and the TB are coupled and a cleavable moiety (CM) is positioned between the MM and the TB; expressing the dual-anchored activatable macromolecule; and recovering the dual-anchored activatable macromolecule, wherein the MM and the TB are tethered at their isopeptide bonding sites in the recovered dual-anchored activatable macromolecule.
49. A method of making a dual-anchored activatable macromolecule comprising providing a MM comprising a non-alpha-carbon covalent bond-forming amino acid configured to form a non-alpha-carbon covalent bond with a non-alpha-carbon covalent bond-forming amino acid in a TB that is coupled to the MM.
50. The method of item 49, wherein the alpha-carbon covalent bond-forming amino acid of the MM is threonine and the alpha-carbon covalent bond-forming amino acid of the TB is glutamine.
51. The method of item 49, wherein the alpha-carbon covalent bond-forming amino acid of the MM is glutamine and the alpha-carbon covalent bond-forming amino acid of the TB is threonine.
52. The method of item 49, wherein the alpha-carbon covalent bond-forming amino acid of the MM is cysteine and the alpha-carbon covalent bond-forming amino acid of the TB is glutamine or tyrosine.
53. The method of item 49, wherein the alpha-carbon covalent bond-forming amino acid of the MM is glutamine or tyrosine and the alpha-carbon covalent bond-forming amino acid of the TB is cysteine.
54. The method of item 49, wherein the alpha-carbon covalent bond-forming amino acid of the MM is histidine and the alpha-carbon covalent bond-forming amino acid of the TB is tyrosine.
55. The method of item 49, wherein the alpha-carbon covalent bond-forming amino acid of the MM is tyrosine and the alpha-carbon covalent bond-forming amino acid of the TB is histidine.
56. The method of item 49, wherein the alpha-carbon covalent bond-forming amino acid of the MM is lysine and the alpha-carbon covalent bond-forming amino acid of the TB is cysteine, glutamate, or aspartate.
57. The method of item 49, wherein the alpha-carbon covalent bond-forming amino acid of the MM is cysteine, glutamate, or aspartate and the alpha-carbon covalent bond-forming amino acid of the TB is lysine.
58. The method of item 49, wherein the alpha-carbon covalent bond-forming amino acid of the MM is glutamine and the alpha-carbon covalent bond-forming amino acid of the TB is lysine, and the non-alpha-carbon covalent bond is an isopeptide bond between the gamma-carboxyamide group of the glutamine and the epsilon-amino group of the lysine sidechain.
59. The method of item 49, wherein the alpha-carbon covalent bond-forming amino acid of the MM is lysine and the alpha-carbon covalent bond-forming amino acid of the TB is glutamine, and the non-alpha-carbon covalent bond is an isopeptide bond between the gamma-carboxyamide group of the glutamine and the epsilon-amino group of the lysine sidechain.
60. A method of making a dual-anchored activatable macromolecule comprising providing a MM comprising a cysteine configured to form a non-alpha-carbon covalent bond with a non-alpha-carbon covalent bond-forming amino acid in a TB that is coupled to the MM.

### EXAMPLES

The invention is further described in the following examples, which do not limit the scope of the invention described in the claims and provide proof-of-concept demonstration for the advantageous of the dual-anchored structure of the activatable macromolecules described in the present disclosure.

### Example 1: Production of Single-Anchored BC2T Masked Nanobody (BC2T-Nb)

This example shows the production of an example single-anchored activatable antibody. The single-anchored activatable antibody comprises a nanobody (Nb) capable of binding to beta-catenin and an MM (BC2T) comprising a sequence of beta-catenin that is the epitope of the Nb. The MM is single-anchored with the Nb, i.e., the MM and the Nb are coupled via a CM but not tethered by a disulfide bond. The single-anchored BC2T-Nb (shown in FIG. 4B) was prepared by recombinant methods. The single-anchored BC2T-Nb (SEQ ID NO:2 or 3) comprises, from N-terminus to C-terminus, a signal peptide from SP5 (SEQ ID NO: 12), a leader sequence (SEQ ID NO: 13), the BC2T MM (SEQ ID NO:14), a GS linker (SEQ ID NO: 15), a CM (SEQ ID NO: 16 or 17), a GS linker (SEQ ID NO:18), an Nb (SEQ ID NO:19), a GGS linker, and a His tag for purification (SEQ ID NO: 21). The polypeptide was prepared by transforming a host cell with a polynucleotide encoding the polypeptide sequence of SEQ ID NO: 2 or 3, followed by cultivation of the resulting recombinant host cells, and purification of the protein from supernatants using standard Immobilized Metal Affinity Chromatography (IMAC) and Size Exclusion Chromatography (SEC) methods. The resulting protein is a monomer (ProC653 or ProC654).

The control molecule (i.e., unmasked nanobody that has no BC2T attached) was also prepared by recombinant methods. ProC649 (PP073) (SEQ ID NO: 1) comprises, from N-terminus to C-terminus, a signal peptide from SP5 (SEQ ID NO:12), an Nb (SEQ ID NO:19), a GGS linker, and a His tag for purification (SEQ ID NO: 21). The polypeptide was prepared by transforming a host cell with a polynucleotide encoding the polypeptide sequence of SEQ ID NO: 1, followed by cultivation of the resulting recombinant host cells and purification using standard IMAC and SEC methods. The resulting protein (ProC649) is a monomer.

### Example 2. Protease Treatment of Single-Anchored BC2T-Nb

To release the MM, the unmasked nanobody and the single-anchored BC2T-Nb molecules were treated overnight at 37° C. with a recombinant human protease such as matrix metalloproteinase (MMP). Complete protease treatment was tested by non-reducing SDS-PAGE. Protein aliquots (2 μg) were denatured for 10 minutes at 75° C. in sample buffer (with reducing agent added, as necessary) and separated on a 4-12% NuPAGE™ Bis-Tris gel (Thermo Fisher Scientific, Waltham, MA, Catalog #NP0321) in MOPS buffer for 1 hour at 175V and visualized after staining with InstantBlue™ for 1 hour followed by destaining in water for at least 4 h.

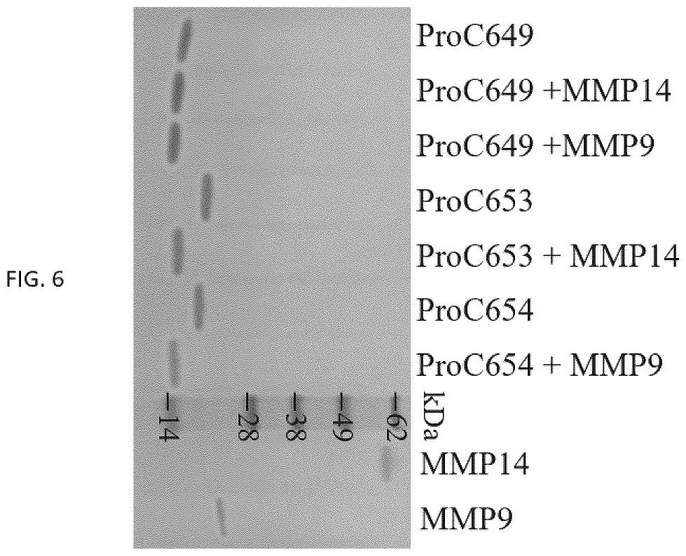

Proteins were confirmed to be monomeric and of the expected molecular weights (FIG. 6). Protease treatment did not affect the integrity of the unmasked molecule (FIG. 6, lanes 1, 2, 3). There is a shift in molecular weight in the constructs with the BC2T and the nanobody expressed as a polypeptide and linked with a CM (FIG. 6, lanes 4, 6). After protease treatment, the proteins run at the same level as the unmasked molecule (FIG. 6, lanes 5, 7).

### Example 3: Binding of Monomeric Single-Anchored BC2T-Nb by BioLayer Interferometry (BLI)

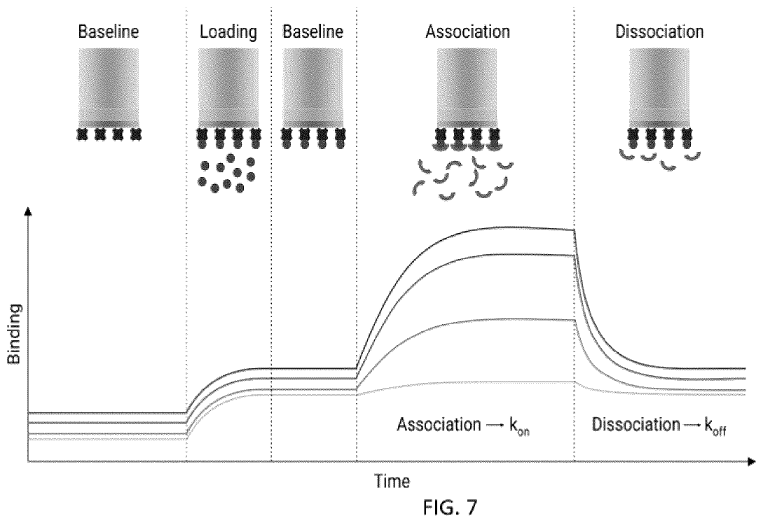

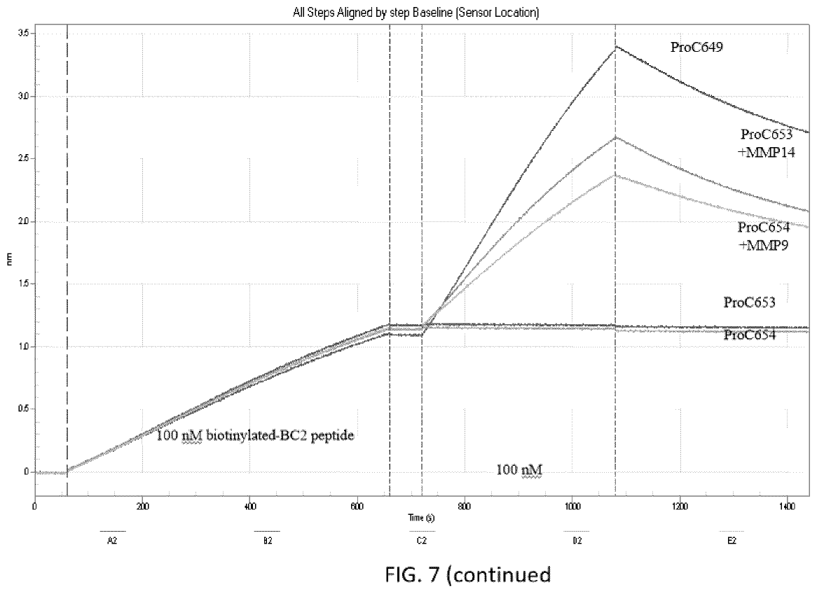

Binding of the unmasked nanobody and single-anchored BC2T-Nb to free BC2T peptide was assessed by BLI using the ForteBio Octet device. While BLI is generally used to calculate association and dissociation kinetics of binding interactions, it was used herein to detect binding Peptide corresponding to the following sequence: QGQSGQPDRKAAVSHWQ (SEQ ID NO: 567) was synthesized at ELIM Biopharmaceuticals with a biotin at the N-terminus. The peptide was loaded on SSA biosensor tips (Pall/ForteBio Cat #18-5117) at 100 nM in Binding Buffer (BB: 1×PBS, pH 7.2, 5% glycerol, 0.1% Tween-20, 2% BSA) for 10 minutes. Baseline incubation in BB for 1 min before and after this capture step were used to confirm protein capture. The loaded tips were then incubated in 100 nM Nb in BB for 6 minutes followed by incubation in BB for 6 minutes. Changes in interference distance (nanometers) at the sensor tip are plotted against time to follow the binding/dissociation of soluble proteins from the immobilized ligand on the tip (FIG. 7). The results demonstrate that ProC653 and ProC654 are effectively masked because they can bind the peptide, like the unmasked control ProC649, only after protease (MMP9 or MMP14) treatment.

### Example 4: Production of Monomeric Dual-Anchored BC2T-Nb

This example shows production of an example monomeric dual-anchored activatable antibody. The dual-anchored activatable antibody comprises a nanobody (Nb) capable of binding to beta-catenin and an MM comprising a sequence of beta-catenin that is the epitope of the Nb (BC2T). The MM is dual-anchored with the Nb, i.e., the MM and the Nb are coupled via a CM and also tethered by a disulfide bond. The dual-anchored BC2T-Nb (shown in FIG. 4A) were prepared by recombinant methods. ProC994 (PP123) (SEQ ID NO: 4) and ProC995 (PP124) (SEQ ID NO:5) comprise, from N-terminus to C-terminus, a signal peptide from SP5 (SEQ ID NO:12), a leader sequence with the third amino acid mutated to a cysteine (SEQ ID NO: 22), the BC2T MM (SEQ ID NO:14), a GS linker (SEQ ID NO: 15), a CM (SEQ ID NO: 16), a GS linker (SEQ ID NO:18), an Nb with one amino acid mutated to a cysteine (SEQ ID NO: 24 or 25), a GGS linker, and His tag for purification (SEQ ID NO: 21). The polypeptide was prepared by transforming a host cell with a polynucleotide encoding the polypeptide sequence of SEQ ID NO: 4 or 5, followed by cultivation of the resulting recombinant host cells. The resulting protein (ProC994 or ProC995) is a monomer.

ProC996 (PP125) (SEQ ID NO:6) comprises, from N-terminus to C-terminus, a signal peptide from SP5 (SEQ ID NO:12), a leader sequence (SEQ ID NO: 13), the BC2T MM with the second amino acid mutated to a cysteine (SEQ ID NO:23), a GS linker (SEQ ID NO: 15), a CM (SEQ ID NO: 16), a GS linker (SEQ ID NO:18), an Nb with one amino acid mutated to a cysteine (SEQ ID NO:26), a GGS linker, and a His tag for purification (SEQ ID NO: 21). The polypeptide was prepared by transforming a host cell with a polynucleotide encoding the polypeptide sequence of SEQ ID NO: 6, followed by cultivation of the resulting recombinant host cells and purification of the expressed protein using standard IMAC and SEC methods. The resulting protein (ProC996) is a monomer.

### Example 5. Protease Treatment of Monomeric Dual-anchored BC2T-Nb

To release the MM (see FIG. 4C), single-anchored and dual-anchored BC2T-Nb molecules were treated overnight at 37° C. with a recombinant human protease such as urokinase-type plasminogen activator (uPA). Complete protease treatment and disulfide bond analysis was tested by reducing and non-reducing SDS-PAGE. Protein aliquots (2 μg) were denatured for 10 minutes at 75° C. in sample buffer (with reducing agent added, as necessary) and separated on a 4-12% NuPAGE™ Bis-Tris gel (Thermo Fisher Scientific, Waltham, MA, Catalog #NP0321) in MOPS buffer for 1 hour at 175V and visualized after staining with InstantBlue™ for 1 hour followed by destaining in water for at least 4 h.

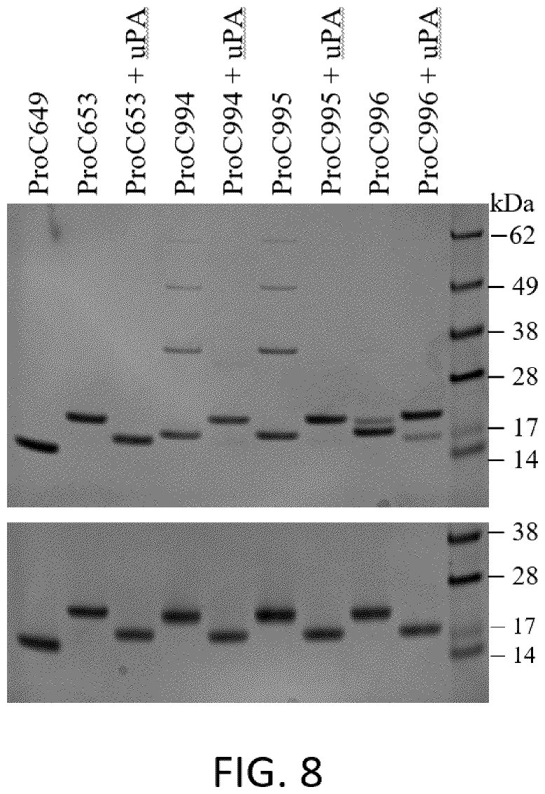

Proteins were confirmed to be monomeric and of the expected molecular weights on the reducing gel before and after protease treatment (FIG. 8, bottom panel). Engineered disulfide bonds were confirmed to have formed in the non-reducing gel before protease treatment (FIG. 8, top panel, lanes 4, 6, and 8), and after protease treatment, the proteins run at the same level as the single-anchored mask molecule (FIG. 8, top panel, lanes 2, 5, 7, 9). Engineered disulfide bonds were confirmed to have formed by comparing the migration of protease-treated dual-anchored masked proteins in non-reducing vs reducing gels.

### Example 6: Production of Dimeric Dual-anchored Macromolecules

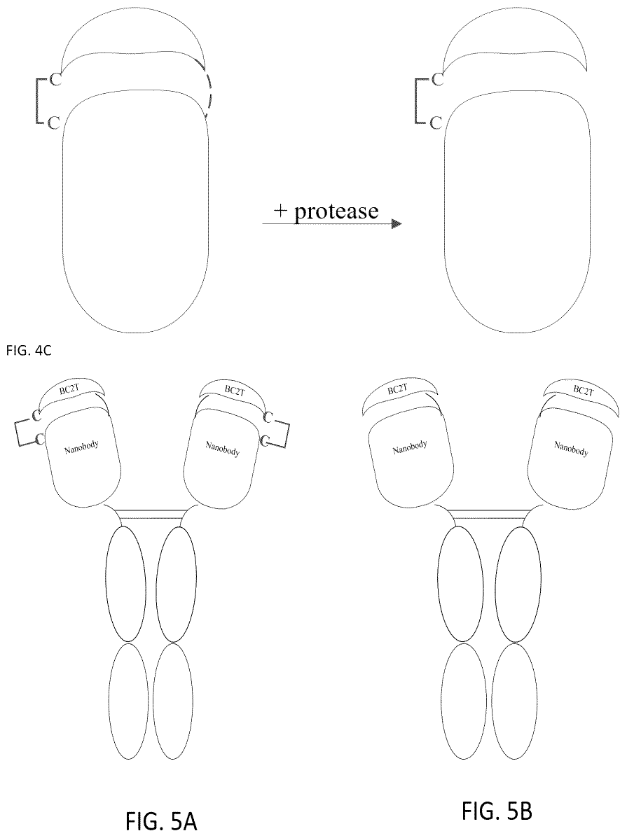

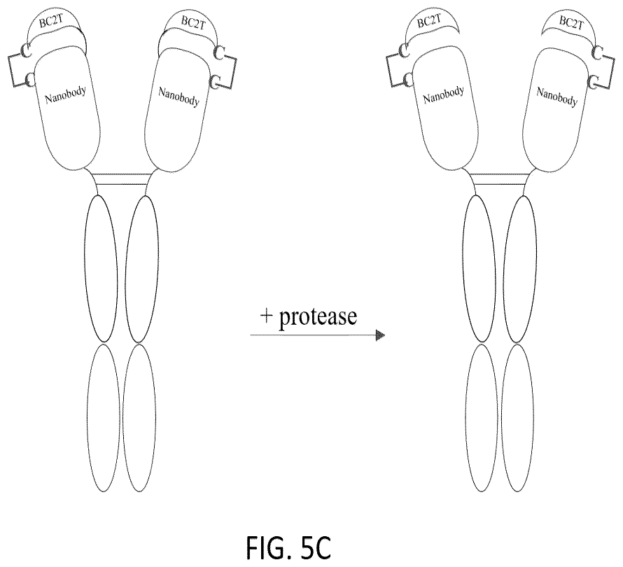

This example shows production of an exemplary dimeric dual-anchored activatable antibody. The dual-anchored activatable molecule includes two nanobodies (Nb) capable of binding to beta-catenin, an Fc dimer comprising two Fe, each coupled with an Nb, and two MMs (e.g., BC2T in this illustrative example) comprising a sequence of beta-catenin that is the epitope of the Nb. Each MM is dual-anchored with the Nb, i.e., the MM and the Nb are coupled via a CM and also tethered by a disulfide bond (FIG. 5A).

The dimeric dual-anchored BC2T-Nb was prepared by recombinant methods. ProC1285 (HC699) (SEQ ID NO: 9) comprises, from N-terminus to C-terminus, a signal peptide from SP5 (SEQ ID NO:12), a leader sequence with the third amino acid mutated to a cysteine (SEQ ID NO.22), the BC2T MM (SEQ ID NO:14), a GS linker (SEQ ID NO: 15), a CM (SEQ ID NO: 16), a GS linker (SEQ ID NO:18), a Nb with one amino acid mutated to a cysteine (SEQ ID NO: 24), a GGGG linker (SEQ ID NO: 27), and the human IgG1 Fc (SEQ ID NO: 28). The polypeptide was prepared by transforming a host cell with a polynucleotide encoding the polypeptide sequence of SEQ ID NO: 9, followed by cultivation of the resulting recombinant host cells and purification of the expressed protein using standard IMAC and SEC methods. The resulting protein (ProC1285) is a dimer (shown in FIG. 5A).

ProC1287 (HC701) (SEQ ID NO:11) comprises, from N-terminus to C-terminus, a signal peptide from SP5 (SEQ ID NO:12), a leader sequence (SEQ ID NO:13), the BC2T MM with the second amino acid mutated to a cysteine (SEQ ID NO:23), a GS linker (SEQ ID NO: 15), a CM (SEQ ID NO: 16), a GS linker (SEQ ID NO:18), an Nb with one amino acid mutated to a cysteine (SEQ ID NO:26), a GGGG linker (SEQ ID NO: 27), and the human IgG1 Fc (SEQ ID NO: 28). The polypeptide was prepared by transforming a host cell with a polynucleotide encoding the polypeptide sequence of SEQ ID NO: 11, followed by cultivation of the resulting recombinant host cells and purification of the expressed protein using standard IMAC and SEC methods. The resulting protein (ProC1287) is a dimer (shown in FIG. 5A).

A single-anchored BC2T-Nb-IgG1 (i.e., the MM and Nb are not tethered by disulfide bond, shown in FIG. 5B) was also prepared by recombinant methods. ProC1284 (HC698) (SEQ ID NO: 8) comprises, from N-terminus to C-terminus, a signal peptide from SP5 (SEQ ID NO: 12), a leader sequence (SEQ ID NO:13), the BC2T MM (SEQ ID NO:14), a GS linker (SEQ ID NO: 15), a CM (SEQ ID NO: 16), another GS linker (SEQ ID NO:18), a Nb (SEQ ID NO: 19), a GGGG linker (SEQ ID NO: 27), and the human IgG1 Fc (SEQ ID NO: 28). The polypeptide was prepared by transforming a host cell with a polynucleotide encoding the polypeptide sequence of SEQ ID NO: 8, followed by cultivation of the resulting recombinant host cells and purification of the expressed protein using standard IMAC and SEC methods. The resulting protein (ProC1284) is a dimer.

### Example 7: Protease Treatment of Dimeric Dual-Anchored BC2T-Nb of Example 6

To release the MM in the molecules of Example 6, single-anchored and dual-anchored BC2T-Nb molecules were treated overnight at 37° C. with a recombinant human protease such as urokinase-type plasminogen activator (uPA) as schematically illustrated in FIG. 5C. Complete protease treatment and disulfide bond analysis was tested by reducing and non-reducing SDS-PAGE. Protein aliquots (2 μg) were denatured for 10 minutes at 75° C. in sample buffer (with reducing agent added, as necessary) and separated on a 4-12% NuPAGE™ Bis-Tris gel (Thermo Fisher Scientific, Waltham, MA, Catalog #NP0321) in MOPS buffer for 1 hour at 175V and visualized after staining with InstantBlue™ for 1 hour followed by destaining in water for at least 4 h.

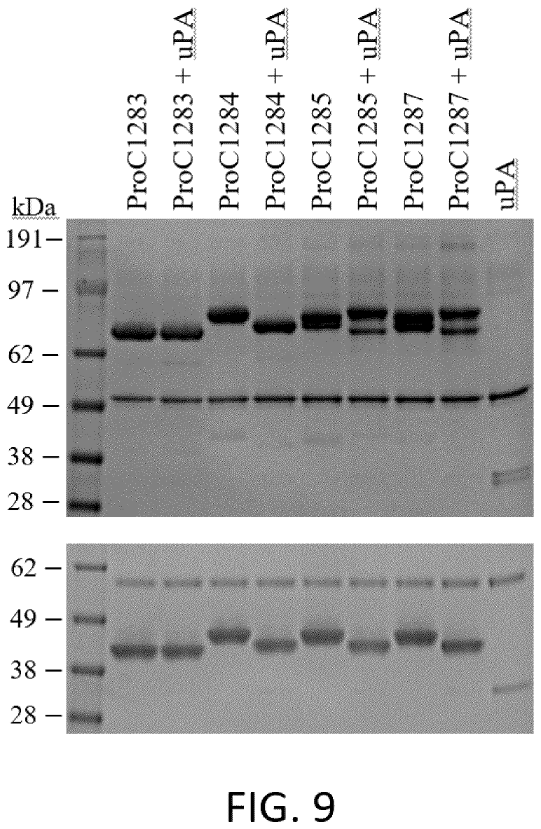

Proteins were confirmed to be monomeric of the expected molecular weights on the reducing gel before and after protease treatment, ˜19 and ˜16 kDa respectively (FIG. 9, bottom panel). Engineered disulfide bonds were confirmed to have formed in the non-reducing gel before protease treatment (FIG. 9, top panel, lanes 4, 6, and 8), and after protease treatment, the proteins run at the same level as the single-anchored mask molecule (FIG. 9, top panel, lanes 2, 5, 7, 9). Engineered disulfide bonds were confirmed to have formed by comparing the migration of protease-treated dual-anchored masked proteins in non-reducing vs reducing gels.

### Example 8: Peptide Binding ELISA

BC2T peptide was synthesized at ELIM Biopharmaceuticals. The peptide sequence comprises, from N-terminus to C-terminus, a leader sequence (SEQ ID NO:13) and the BC2T MM (SEQ ID NO:14).

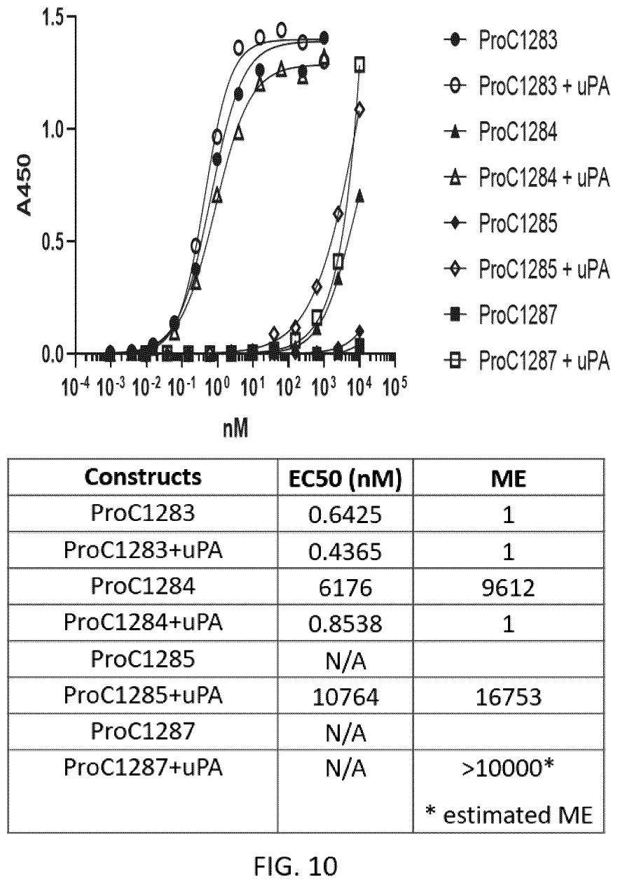

5 μM of the BC2T peptide dissolved in 0.05M carbonate-bicarbonate buffer was adsorbed to the wells of a 96-well micro-titer plate overnight at 4° C. Plates were washed and blocked with blocking buffer (1×PBS, pH 7.4, 0.05% Tween-20, 1% BSA). Three-fold serial dilutions were made of the dimeric dual-anchored mask molecules (ProC1285 and ProC1287) without or with protease treatment along with the unmasked control protein (ProC1283) and the dimeric single-anchored BC2T-Nb (ProC1284) and applied to the peptide-coated plate. The extent of protein bound to the peptide was measured by anti-human-IgG immune-detection A450 absorbance was measured on the plate reader. Dose-response curves were generated and EC50 values were obtained by sigmoidal fit non-linear regression using Graph Pad Prism software. The results are shown in FIG. 10. As shown in FIG. 10, the dimeric dual-anchored mask molecules (ProC1285 and ProC1287) exhibited much greater masking compared to the comparative dimeric single-anchored BC2T-Nb (ProC1284), and also demonstrated a surprisingly large degree of recovery of binding activity upon cleavage compared to the recovery exhibited by the positive control ProC1283 (unmasked antibody). Thus, despite being tethered at one end to a mask after cleavage, the antibodies demonstrated a substantial recovery of binding activity.

### Example 9: Designing a Disulfide-Based Dual-Anchored-Masked Antibody

This example describes how one may engineer non-alpha-carbon linkages, for example a cysteine-disulfide bond, between a residue in the prodomain (N-terminal to the mask peptide) and a residue in the antibody variable domains using an activatable anti-PDL1 antibody as an example. The sequence of the activatable anti-PDL1 antibody (SEQ ID NOS: 562 and 563) consists of a prodomain connected to the light chain of a canonical antibody. The antibody primary sequence was used to generate a homology-based three-dimensional model of the antibody using software like Discovery Studio (FIGS. 12A-12B). The mask sequence from the prodomain was used to generate homology-based models of the mask and docked into the antibody structure (FIGS. 12A-12B). FIG. 12A illustrates the three-dimensional structure of the activatable anti-PDL1 antibody obtained using BIOVIA Discovery Studios from Dassault Systemes software showing solvent accessible residues within 2-5 angstroms of residues in the header region or N-terminus of the mask moiety. FIG. 12B shows the interface between the Fab domain (space-filling format) and the Prodomain with mask moiety (shown as the Cα backbone) of the activatable anti-PDL 1 antibody.

A variety of experimental techniques, including mutagenesis, Hydrogen-Deuterium Exchange (HDX), and XL-MS (cross-linker MS), may be used optionally to confirm the structural model. After obtaining a three-dimensional model for the MM bound to the TB, energy minimization algorithms are used to provide information about the location of the header and linker (including CM) regions of the prodomain. The user can then use software, like BIOVIA Discovery Studio, to identify pairs of residues, one of which lies in the header region of the prodomain and the other of which lies in the antibody variable domain, whose Cα atoms are within the 3-7.5 Å range (i.e., the distance between the Cα of the first amino acid and the Cα of the second amino acid) of canonical disulfide-linked cysteines. Software applications, such as SSBondPre, may be used to refine this list to a smaller list of residues that have a high likelihood of forming designed disulfide bonds. The residues are then mutated to cysteine residues, individually and in pairs, to identify a pair of residues that individually do not affect binding of the antibody or the mask but that when present together form a disulfide bond that improves masking. The presence of the correctly formed disulfide may be confirmed by mass spectrometry techniques such as disulfide mapping.

### Example 10: Designing Disulfide-Based Dual-Anchored-Masked Antibodies or Other TBs

This example provides further examples of how one may prepare and use homology-based three-dimensional models of antibody structures in order to engineer non-alpha-carbon linkages, for example a cysteine-disulfide bond, between a residue in the prodomain (N-terminal to the mask peptide) and a residue in the antibody variable domains.

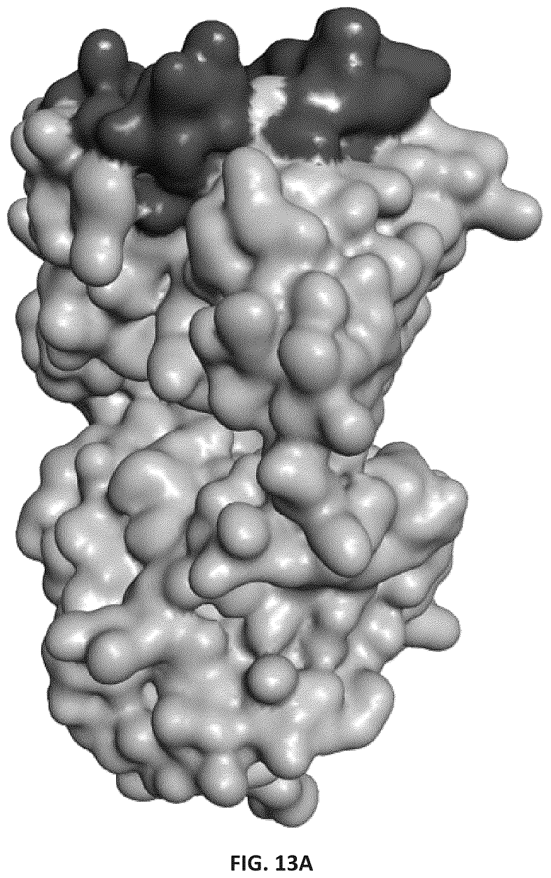

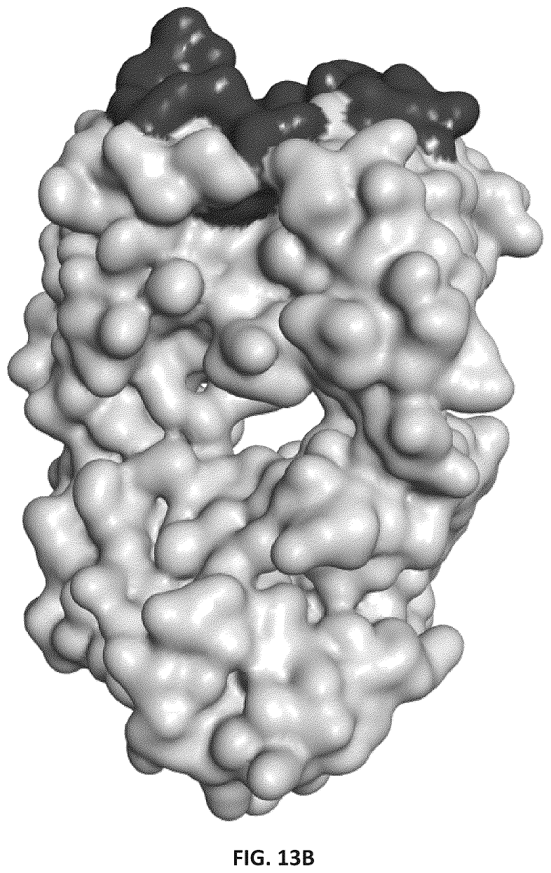

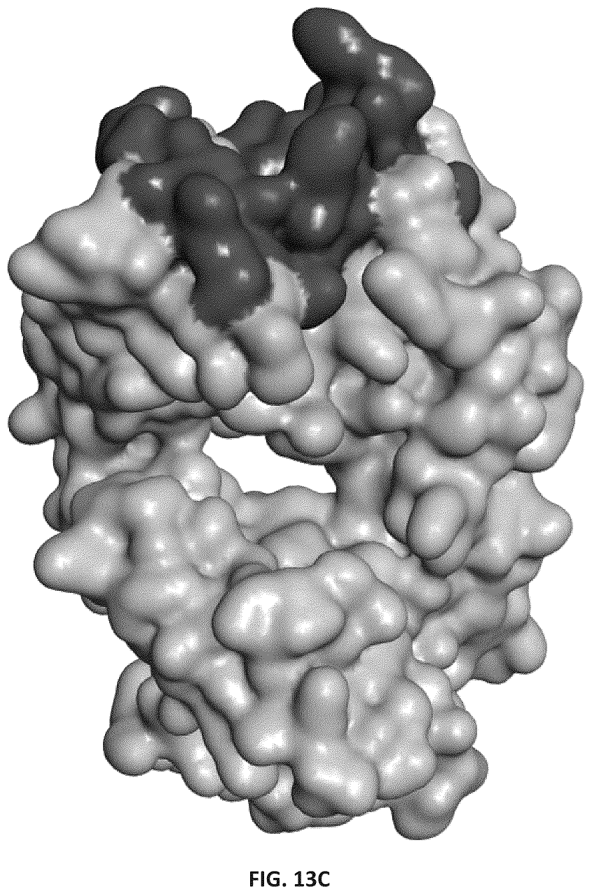

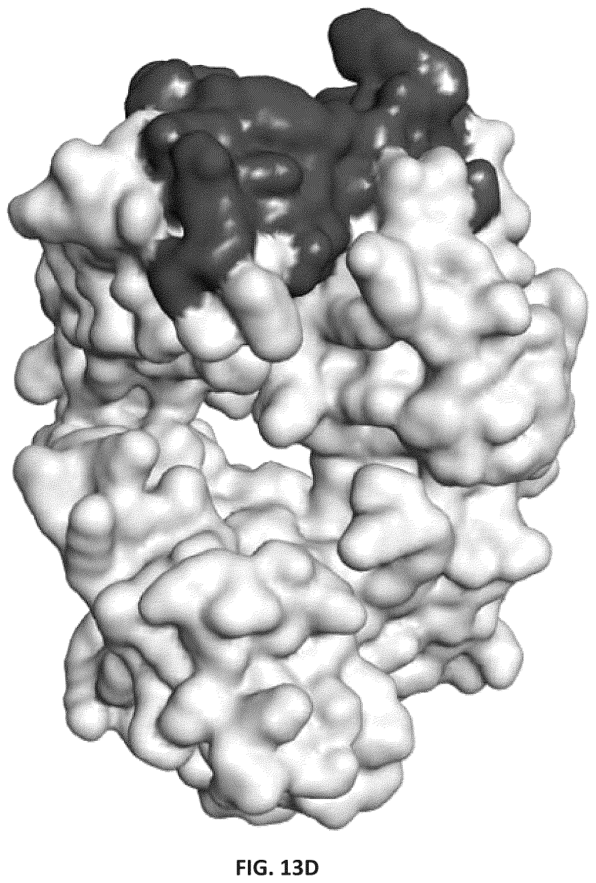

BIOVIA Discovery Studio was used to prepare homology-based three-dimensional models of antibody structures corresponding to the sequences for J43v2/anti-mouse PD1 (FIG. 13A; SEQ ID NOs. 568-569), anti-CD166 (FIG. 13B, SEQ ID NOs: 572-573), and human anti-PD1 (FIG. 13C; SEQ ID NOs: 570-571). The anti-PD1 human anti-PD1 (SEQ ID NOs: 570-571) was additionally modelled in AlphaFold2 and rendered in BIOVIA Discovery Studio as shown in FIG. 13D.

A variety of experimental techniques, including mutagenesis, Hydrogen-Deuterium Exchange (HDX), and XL-MS (cross-linker MS), may be used optionally to confirm the structural model. MMs for the modeled human anti-PD1 are disclosed in WO2017/011580 and MMs for the modeled anti-CD166 are disclosed in WO2016/179285, both of which are incorporated herein by reference. One can obtain a three-dimensional model for the respective MM bound to the respective TB, and use software, like BIOVIA Discovery Studio, to identify pairs of residues, one of which lies in the header region of the prodomain and the other of which lies in the antibody variable domain, whose Cα atoms are within the 3-7.5 Å range (i.e., the distance between the Calpha of the first amino acid and the Calpha of the second amino acid). Software applications, such as SSBondPre, may be used to refine this list to a smaller list of residues that have a high likelihood of forming the desired bonds. The residues are then mutated to the desired residues to form the desired bonds, individually and in pairs, to identify a pair of residues that individually do not affect binding of the antibody or the mask but that when present together form a bond that improves masking as described herein. The presence of the correctly formed bonds, e.g., disulfide bonds, may be confirmed by mass spectrometry techniques such as disulfide mapping.

The sequences of the molecules in the examples are listed in Table 4 below.

TABLE 4

| SEQ ID NO. | NAME/NOTES | SEQUENCE |
|---|---|---|
| 1 | ProC649/PP073 | QVQLVESGGGLVQPGGSLTLSCTASGFTLDHYDIGWF |
| | Nanobody (with | RQAPGKEREGVSCINNSDDDTYYADSVKGRFTIFMNN |
| | His tag) | AKDTVYLQMNSLKPEDTAIYYCAEARGCKRGRYEYDF |
| | | WGQGTQVTVSSGGSHHHHHHHHHH |
| 2 | ProC653/PP077 | QGQSGQPDRKAAVSHWQQGGGSSGGSISSGLLSGRSD |
| | BC2 tag-1490DNI- | NIGGGSQVQLVESGGGLVQPGGSLTLSCTASGFTLDHY |
| | Nanobody | DIGWFRQAPGKEREGVSCINNSDDDTYYADSVKGRFTI |
| | | FMNNAKDTVYLQMNSLKPEDTAIYYCAEARGCKRGR |
| | | YEYDFWGQGTQVTVSSGGSHHHHHHHHHH |
| 3 | ProC654/PP078 | QGQSGQPDRKAAVSHWQQGGGSSGGSPLGLAGGGGS |
| | BC2 tag-PLGLAG- | QVQLVESGGGLVQPGGSLTLSCTASGFTLDHYDIGWF |
| | Nanobody | RQAPGKEREGVSCINNSDDDTYYADSVKGRFTIFMNN |
| | | AKDTVYLQMNSLKPEDTAIYYCAEARGCKRGRYEYDF |
| | | WGQGTQVTVSSGGSHHHHHHHHHH |
| 4 | ProC994/PP123 | QGCSGQPDRKAAVSHWQQGGGSSGGSISSGLLSGRSD |
| | Q3C Q157C BC2 | NIGGGSQVQLVESGGGLVQPGGSLTLSCTASGFTLDHY |
| | tag-1490DNI- | DIGWFRQAPGKEREGVSCINNSDDDTYYADSVKGRFTI |
| | Nanobody | FMNNAKDTVYLQMNSLKPEDTAIYYCAEARGCKRGR |
| | | YEYDFWGCGTQVTVSSGGSHHHHHHHHHH |
| 5 | ProC995/PP124 | QGCSGQPDRKAAVSHWQQGGGSSGGSISSGLLSGRSD |
| | Q3C W155C BC2 | NIGGGSQVQLVESGGGLVQPGGSLTLSCTASGFTLDHY |
| | tag-1490DNI- | DIGWFRQAPGKEREGVSCINNSDDDTYYADSVKGRFTI |
| | Nanobody | FMNNAKDTVYLQMNSLKPEDTAIYYCAEARGCKRGR |
| | | YEYDFCGQGTQVTVSSGGSHHHHHHHHHH |
| 6 | ProC996/PP125 | QGQSGQPCRKAAVSHWQQGGGSSGGSISSGLLSGRSD |
| | D8C F154C BC2 | NIGGGSQVQLVESGGGLVQPGGSLTLSCTASGFTLDHY |
| | tag-1490DNI- | DIGWFRQAPGKEREGVSCINNSDDDTYYADSVKGRFTI |
| | Nanobody | FMNNAKDTVYLQMNSLKPEDTAIYYCAEARGCKRGR |
| | | YEYDCWGQGTQVTVSSGGSHHHHHHHHHH |
| 7 | ProC1283/HC697 | QVQLVESGGGLVQPGGSLTLSCTASGFTLDHYDIGWF |
| | Nanobody-IgG1 | RQAPGKEREGVSCINNSDDDTYYADSVKGRFTIFMNN |
| | | AKDTVYLQMNSLKPEDTAIYYCAEARGCKRGRYEYDF |
| | | WGQGTQVTVSSGGGGDKTHTCPPCPAPELLGGPSVFL |
| | | FPPKPKDTLMISRTPEVTCVVVDVSHEDPEVKFNWYV |
| | | DGVEVHNAKTKPREEQYNSTYRVVSVLTVLHQDWLN |
| | | GKEYKCKVSNKALPAPIEKTISKAKGQPREPQVYTLPP |
| | | SREEMTKNQVSLTCLVKGFYPSDIAVEWESNGQPENN |
| | | YKTTPPVLDSDGSFFLYSKLTVDKSRWQQGNVFSCSV |
| | | MHEALHNHYTQKSLSLSPGK |
| 8 | ProC1284/HC698 | QGQSGQPDRKAAVSHWQQGGGSSGGSISSGLLSGRSD |
| | BC2 tag-1490DNI- | NIGGGSQVQLVESGGGLVQPGGSLTLSCTASGFTLDHY |
| | Nanobody-IgG1 | DIGWFRQAPGKEREGVSCINNSDDDTYYADSVKGRFTI |
| | | FMNNAKDTVYLQMNSLKPEDTAIYYCAEARGCKRGR |
| | | YEYDFWGQGTQVTVSSGGGGDKTHTCPPCPAPELLGG |
| | | PSVFLFPPKPKDTLMISRTPEVTCVVVDVSHEDPEVKFN |
| | | WYVDGVEVHNAKTKPREEQYNSTYRVVSVLTVLHQD |
| | | WLNGKEYKCKVSNKALPAPIEKTISKAKGQPREPQVY |
| | | TLPPSREEMTKNQVSLTCLVKGFYPSDIAVEWESNGQP |
| | | ENNYKTTPPVLDSDGSFFLYSKLTVDKSRWQQGNVFS |
| | | CSVMHEALHNHYTQKSLSLSPGK |
| 9 | ProC1285/HC699 | QGCSGQPDRKAAVSHWQQGGGSSGGSISSGLLSGRSD |
| | Q3C Q157C BC2 | NIGGGSQVQLVESGGGLVQPGGSLTLSCTASGFTLDHY |
| | tag-1490DNI-Nb- | DIGWFRQAPGKEREGVSCINNSDDDTYYADSVKGRFTI |
| | IgG1 | FMNNAKDTVYLQMNSLKPEDTAIYYCAEARGCKRGR |
| | | YEYDFWGCGTQVTVSSGGGGDKTHTCPPCPAPELLGG |
| | | PSVFLFPPKPKDTLMISRTPEVTCVVVDVSHEDPEVKFN |
| | | WYVDGVEVHNAKTKPREEQYNSTYRVVSVLTVLHQD |
| | | WLNGKEYKCKVSNKALPAPIEKTISKAKGQPREPQVY |
| | | TLPPSREEMTKNQVSLTCLVKGFYPSDIAVEWESNGQP |
| | | ENNYKTTPPVLDSDGSFFLYSKLTVDKSRWQQGNVFS |
| | | CSVMHEALHNHYTQKSLSLSPGK |
| 10 | ProC1286/HC700 | QGCSGQPDRKAAVSHWQQGGGSSGGSISSGLLSGRSD |
| | Q3C W155C BC2 | NIGGGSQVQLVESGGGLVQPGGSLTLSCTASGFTLDHY |
| | tag-1490DNI-Nb- | DIGWFRQAPGKEREGVSCINNSDDDTYYADSVKGRFTI |
| | IgG1 | FMNNAKDTVYLQMNSLKPEDTAIYYCAEARGCKRGR |
| | | YEYDFCGQGTQVTVSSGGGGDKTHTCPPCPAPELLGG |
| | | PSVFLFPPKPKDTLMISRTPEVTCVVVDVSHEDPEVKFN |
| | | WYVDGVEVHNAKTKPREEQYNSTYRVVSVLTVLHQD |
| | | WLNGKEYKCKVSNKALPAPIEKTISKAKGQPREPQVY |
| | | TLPPSREEMTKNQVSLTCLVKGFYPSDIAVEWESNGQP |
| | | ENNYKTTPPVLDSDGSFFLYSKLTVDKSRWQQGNVFS |
| | | CSVMHEALHNHYTQKSLSLSPGK |
| 11 | ProC1287/HC701 | QGQSGQPCRKAAVSHWQQGGGSSGGSISSGLLSGRSD |
| | D8C F154C BC2 | NIGGGSQVQLVESGGGLVQPGGSLTLSCTASGFTLDHY |
| | tag-1490DNI-Nb- | DIGWFRQAPGKEREGVSCINNSDDDTYYADSVKGRFTI |
| | IgG1 | FMNNAKDTVYLQMNSLKPEDTAIYYCAEARGCKRGR |
| | | YEYDCWGQGTQVTVSSGGGGDKTHTCPPCPAPELLGG |
| | | PSVFLFPPKPKDTLMISRTPEVTCVVVDVSHEDPEVKFN |
| | | WYVDGVEVHNAKTKPREEQYNSTYRVVSVLTVLHQD |
| | | WLNGKEYKCKVSNKALPAPIEKTISKAKGQPREPQVY |
| | | TLPPSREEMTKNQVSLTCLVKGFYPSDIAVEWESNGQP |
| | | ENNYKTTPPVLDSDGSFFLYSKLTVDKSRWQQGNVFS |
| | | CSVMHEALHNHYTQKSLSLSPGK |
| 12 | SP5 signal peptide | MRAWIFFLLCLAGRALA |
| 13 | Header sequence | QGQSGQ |
| 14 | BC2T | PDRKAAVSHWQQ |
| 15 | Linker 1 | GGGSSGGS |
| 16 | 1490DNI | ISSGLLSGRSDNI |
| 17 | MMP-cleavable | PLGLAG |
| | moiety | |
| 18 | Linker 2 | GGGS |
| 19 | Nanobody | QVQLVESGGGLVQPGGSLTLSCTASGFTLDHYDIGWF |
| | | RQAPGKEREGVSCINNSDDDTYYADSVKGRFTIFMNN |
| | | AKDTVYLQMNSLKPEDTAIYYCAEARGCKRGRYEYDF |
| | | WGQGTQVTVSS |
| | Linker 3 | GGS |
| 21 | His tag | HHHHHHHHHH |
| 22 | Q3C | QGCSGQ |
| 23 | D8C | PCRKAAVSHWQQ |
| 24 | Q157C Nanobody | QVQLVESGGGLVQPGGSLTLSCTASGFTLDHYDIGWF |
| | | RQAPGKEREGVSCINNSDDDTYYADSVKGRFTIFMNN |
| | | AKDTVYLQMNSLKPEDTAIYYCAEARGCKRGRYEYDF |
| | | WGCGTQVTVSS |
| 25 | W155C Nanobody | QVQLVESGGGLVQPGGSLTLSCTASGFTLDHYDIGWF |
| | | RQAPGKEREGVSCINNSDDDTYYADSVKGRFTIFMNN |
| | | AKDTVYLQMNSLKPEDTAIYYCAEARGCKRGRYEYDF |
| | | CGQGTQVTVSS |
| 26 | F154C Nanobody | QVQLVESGGGLVQPGGSLTLSCTASGFTLDHYDIGWF |
| | | RQAPGKEREGVSCINNSDDDTYYADSVKGRFTIFMNN |
| | | AKDTVYLQMNSLKPEDTAIYYCAEARGCKRGRYEYD |
| | | CWGQGTQVTVSS |
| 27 | Linker 4 | GGGG |
| 28 | Human IgG1 Fc | DKTHTCPPCPAPELLGGPSVFLFPPKPKDTLMISRTPEV |
| | | TCVVVDVSHEDPEVKFNWYVDGVEVHNAKTKPREEQ |
| | | YNSTYRVVSVLTVLHQDWLNGKEYKCKVSNKALPAPI |
| | | EKTISKAKGQPREPQVYTLPPSREEMTKNQVSLTCLVK |
| | | GFYPSDIAVEWESNGQPENNYKTTPPVLDSDGSFFLYS |
| | | KLTVDKSRWQQGNVFSCSVMHEALHNHYTQKSLSLSP |
| | | GK |
| 29 | ProC650/PP074 | GGSPDRKAAVSHWQQGGGGSGGGGSQVQLVESGGGL |
| | BC2 tag-10mr- | VQPGGSLTLSCTASGFTLDHYDIGWFRQAPGKEREGVS |
| | Nanobody | CINNSDDDTYYADSVKGRFTIFMNNAKDTVYLQMNSL |
| | | KPEDTAIYYCAEARGCKRGRYEYDFWGQGTQVTVSSG |
| | | GSHHHHHHHHHH |
| 30 | ProC651/PP075 | GGSPDRKAAVSHWQQGGGGSGGGGSGGGGSQVQLVE |
| | BC2 tag-15mr- | SGGGLVQPGGSLTLSCTASGFTLDHYDIGWFRQAPGKE |
| | Nanobody | REGVSCINNSDDDTYYADSVKGRFTIFMNNAKDTVYL |
| | | QMNSLKPEDTAIYYCAEARGCKRGRYEYDFWGQGTQ |
| | | VTVSSGGSHHHHHHHHHH |
| 31 | ProC652/PP076 | GGSPDRKAAVSHWQQGGGGSGGGGSGGGGSGGGGS |
| | BC2 tag-20mr- | QVQLVESGGGLVQPGGSLTLSCTASGFTLDHYDIGWF |
| | Nanobody | RQAPGKEREGVSCINNSDDDTYYADSVKGRFTIFMNN |
| | | AKDTVYLQMNSLKPEDTAIYYCAEARGCKRGRYEYDF |
| | | WGQGTQVTVSSGGSHHHHHHHHHH |
| 562 | Heavy Chain | EVQLLESGGGLVQPGGSLRLSCAASGFTFSSYAMSWV |
| | (activatable anti- | RQAPGKGLEWVSSIWRNGIVTVYADSVKGRFTISRDNS |
| | PDL1 antibody) | KNTLYLQMNSLRAEDTAVYYCAKWSAAFDYWGQGT |
| | | LVTVSSASTKGPSVFPLAPCSRSTSESTAALGCLVKDYF |
| | | PEPVTVSWNSGALTSGVHTFPAVLQSSGLYSLSSVVTV |
| | | PSSSLGTKTYTCNVDHKPSNTKVDKRVESKYGPPCPPC |
| | | PAPEFLGGPSVFLFPPKPKDTLMISRTPEVTCVVVDVSQ |
| | | EDPEVQFNWYVDGVEVHNAKTKPREEQFNSTYRVVS |
| | | VLTVLHQDWLNGKEYKCKVSNKGLPSSIEKTISKAKG |
| | | QPREPQVYTLPPSQEEMTKNQVSLTCLVKGFYPSDIAV |
| | | EWESNGQPENNYKTTPPVLDSDGSFFLYSRLTVDKSR |
| | | WQEGNVFSCSVMHEALHNHYTQKSLSLSLG |
| 563 | activatable anti- | QGQSGSGIALCPSHFCQLPQTGGGSSGGSGGSGGISSGL |
| | PDL1 antibody | LSGRSDNHGGSDIQMTQSPSSLSASVGDRVTITCRASQS |
| | (mask + substrate + | ISSYLNWYQQKPGKAPKLLIYAASSLQSGVPSRFSGSGS |
| | anti-PDL1 antibody | GTDFTLTISSLQPEDFATYYCQQDNGYPSTFGGGTKVEI |
| | light Chain and | KRTVAAPSVFIFPPSDEQLKSGTASVVCLLNNFYPREA |
| | heavy chain) | KVQWKVDNALQSGNSQESVTEQDSKDSTYSLSSTLTL |
| | | SKADYEKHKVYACEVTHQGLSSPVTKSFNRGEC |
| 564 | anti-PDL1 antibody | DIQMTQSPSSLSASVGDRVTITCRASQSISSYLNWYQQ |
| | Light Chain | KPGKAPKLLIYAASSLQSGVPSRFSGSGSGTDFTLTISSL |
| | | QPEDFATYYCQQDNGYPSTFGGGTKVEIKRTVAAPSVF |
| | | IFPPSDEQLKSGTASVVCLLNNFYPREAKVQWKVDNA |
| | | LQSGNSQESVTEQDSKDSTYSLSSTLTLSKADYEKHKV |
| | | YACEVTHQGLSSPVTKSFNRGEC |
| 565 | activatable anti- | GIALCPSHFCQLPQT |
| | PDL1 antibody | |
| | Mask | |
| 79 | activatable anti- | ISSGLLSGRSDNH |
| | PDL1 antibody CM | |
| 566 | activatable anti- | QGQSGS |
| | PDL1 antibody | |
| | Spacer | |
| 568 | J43v2 mouse anti- | YELTQPPSASVNVGETVKITCSGDQLPKYFADWFHQRS |
| | PDL1 antibody | DQTILQVIYDDNKRPSGIPERISGSSSGTTATLTIRDVRA |
| | Light Chain | EDEGDYYCFSGYVDSDSKLYVFGSGTQLTVLGGPKSSP |
| | | KVTVFPPSPEELRTNKATLVCLVNDFYPGSATVTWKA |
| | | NGATINDGVKTTKPSKQGQNYMTSSYLSLTADQWKSH |
| | | NRVSCQVTHEGETVEKSLSPAECL |
| 569 | J43v2 mouse anti- | EVRLLESGGGLVKPEGSLKLSCVASGFTFSDYFMSWV |
| | PDL1 antibody | RQAPGKGLEWVAHIYTKSYNYATYYSGSVKGRFTISR |
| | Heavy Chain | DDSRSMVYLQMNNLRTEDTATYYCTRDGSGYPSLDF |
| | | WGQGTQVTVSSAKTTAPSVYPLAPVCGDTTGSSVTLG |
| | | CLVKGYFPEPVTLTWNSGSLSSGVHTFPAVLQSDLYTL |
| | | SSSVTVTSSTWPSQSITCNVAHPASSTKVDKKI |
| 570 | Human anti-PD1 | DIQLTQSPSSLSASVGDRVTITCRASESVDAYGISFMNW |
| | Light Chain | FQQKPGKAPKLLIYAASNQGSGVPSRFSGSGSGTDFTL |
| | | TISSMQPEDFATYYCQQSKDVPWTFGQGTKLEIKRTVA |
| | | APSVFIFPPSDEQLKSGTASVVCLLNNFYPREAKVQWK |
| | | VDNALQSGNSQESVTEQDSKDSTYSLSSTLTLSKADYE |
| | | KHKVYACEVTHQGLSSPVTKSFNRGEC |
| 571 | Human anti-PD1 | EVQLVESGGGLVQPGGSLRLSCAASGFTFSGYAMSWV |
| | Heavy Chain | RQAPGKGLEWVAYISNSGGNAHYADSVKGRFTISRDN |
| | | SKNTLYLQMNSLRAEDTAVYYCTREDYGTSPFVYWG |
| | | QGTLVTVSSASTKGPSVFPLAPCSRSTSESTAALGCLVK |
| | | DYFPEPVTVSWNSGALTSGVHTFPAVLQSSGLYSLSSV |
| | | VTVPSSSLGTKTYTCNVDHKPSNTKVDKRV |
| 572 | Human alpha- | QITLKESGPTLVKPTQTLTLTCTFSGFSLSTYGMGVGWI |
| | CD166 Heavy | RQPPGKALEWLANIWWSEDKHYSPSLKSRLTITKDTSK |
| | Chain | NQVVLTITNVDPVDTATYYCVQIDYGNDYAFTYWGQ |
| | | GTLVTVSSASTKGPSVFPLAPSSKSTSGGTAALGCLVK |
| | | DYFPEPVTVSWNSGALTSGVHTFPAVLQSSGLYSLSSV |
| | | VTVPSSSLGTQTYICNVNHKPSNTKVDKKVEPKSCDKT |
| | | HTCPPCPAPELLGGPSVFLFPPKPKDTLMISRTPEVTCV |
| | | VVDVSHEDPEVKFNWYVDGVEVHNAKTKPREEQYNS |
| | | TYRVVSVLTVLHQDWLNGKEYKCKVSNKALPAPIEKT |
| | | ISKAKGQPREPQVYTLPPSREEMTKNQVSLTCLVKGFY |
| | | PSDIAVEWESNGQPENNYKTTPPVLDSDGSFFLYSKLT |
| | | VDKSRWQQGNVFSCSVMHEALHNHYTQKSLSLSPG |
| 573 | Human alpha- | DIVMTQSPLSLPVTPGEPASISCRSSKSLLHSNGITYLY |
| | CD166 Light Chain | WYLQKPGQSPQLLIYQMSNLASGVPDRFSGSGSGTDFT |
| | | LKISRVEAEDVGVYYCAQNLELPYTFGQGTKLEIKRTV |
| | | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition section headings, the materials, methods, and examples are illustrative only and not intended to be limiting.

What is claimed is:

1. A dual-anchored activatable target-binding protein comprising:
a target-binding protein (TB) that specifically binds to a target;
a masking moiety (MM) coupled to the TB, wherein the MM inhibits binding of the AB to the target, and
a cleavable moiety (CM) coupled to the TB and positioned between the TB and the MM, wherein the CM is a polypeptide that functions as a substrate for a protease, and
further comprising a non-alpha-carbon covalent bond tethering the MM and the TB.

2. The activatable target-binding protein of claim 1, wherein the TB is an antigen-binding protein (AB).

3. The activatable target-binding protein of claim 1 or claim 2, wherein the activatable target-binding protein has a lower target-binding activity compared to a single-anchored activatable target-binding protein lacking the non-alpha-carbon covalent bond.

4. The activatable target-binding protein of any one of claims 1-3, wherein the non-alpha-carbon covalent bond is an ester bond or a thioester bond.

5. The activatable target-binding protein of claim 4, wherein the ester bond is between a threonine and a glutamine.

6. The activatable target-binding protein of claim 4, wherein the thioester bond is between a cysteine and a glutamine or a tyrosine.

7. The activatable target-binding protein of any one of claims 1-3, wherein the non-alpha-carbon covalent bond is a cross-link between a histidine and a tyrosine or a cross-link between a lysine and a cysteine.

8. The activatable target-binding protein of any one of claims 1-3, wherein the non-alpha-carbon covalent bond is an isopeptide bond.

9. The activatable target-binding protein of claim 8, wherein the isopeptide bond is between a lysine and a glutamate or aspartate residue.

10. The activatable target-binding protein of claim 8, wherein the isopeptide bond is between a gamma-carboxyamide group of glutamine and epsilon-amino group of a lysine sidechain.

11. The activatable target-binding protein of any one of claims 1-3, wherein the non-alpha-carbon covalent bond is a disulfide bond.

12. The activatable target-binding protein of claim 11, wherein the disulfide bond is formed between a first cysteine and a second cysteine, wherein
the first cysteine is within the MM and the second cysteine is within the TB,
the first cysteine is within a peptide coupled to the MM and the second cysteine is within the TB, or
the first cysteine is within the MM and the second cysteine is within a peptide coupled to the TB.

13. The activatable target-binding protein of any one of claims 1-12, further comprising a second CM, wherein
the second CM is positioned between the MM and the non-alpha-carbon covalent bond,
the second CM is within the MM and up to 5 amino acids away from a cysteine forming the non-alpha-carbon covalent bond, or
the second CM is within the TB and at up to 5 amino acids away from a cysteine forming the non-alpha-carbon covalent bond.

14. The activatable target-binding protein of any one of claims 1-13, wherein the first and the second CMs are substrates of different proteases.

15. The activatable target-binding protein of any one of claims 1-13, wherein the first and the second CMs are substrates of the same protease.

16. The activatable target-binding protein of any one of claims 1-12, wherein the protease is produced by a tumor in a subject.

17. The activatable target-binding protein of any one or combination of claims 2-16, wherein the AB is an antibody, a Fab fragment, a F(ab′)₂ fragment, a scFv, a scAb, a dAb, a VHH, or a single domain antibody.

18. The activatable target-binding protein of any one of claims 2-17, wherein the AB is a single domain antibody.

19. The activatable target-binding protein of any one of claims 2-18, wherein the AB is an Fc-tagged single domain antibody.

20. The activatable target-binding protein of any one of claims 2-17, wherein the AB is a bispecific antibody.

21. The activatable target-binding protein of claim 20, wherein the bispecific antibody is a bispecific T Cell engager (BiTE) or a dual-affinity retargeting antibody (DART).

22. The activatable target-binding protein of any one of claims 2-17, wherein the AB is a multispecific antibody.

23. The activatable target-binding protein of any one of claims 18-19, wherein the non-alpha-carbon covalent bond is between the MM and the single domain antibody.

24. The activatable target-binding protein of any one of claims 1-22, wherein the non-alpha-carbon covalent bond is between the MM and an Fc domain.

25. The activatable target-binding protein of any one of claims 1-24, wherein the MM comprises an epitope of the TB.

26. The activatable target-binding protein of any one of claims 1-24, wherein the MM does not comprise four or more consecutive amino acids of an epitope of the TB.

27. The activatable target-binding protein of any one of claims 1-26, wherein the MM has a dissociation constant for binding to the TB that is greater than a dissociation constant of the TB for binding to the target.

28. The activatable target-binding protein of any one of claims 1-27, wherein the MM is a polypeptide of from 2 to 40 amino acids in length.

29. The activatable target-binding protein of any one of claims 1-28, wherein the activatable target-binding protein comprises a linker between the MM and the CM.

30. The activatable target-binding protein of any one of claims 1-29, wherein the activatable target-binding protein comprises a linker between CM and the TB.

31. The activatable target-binding protein of any one of claims 1-29, wherein the activatable target-binding protein comprises a first linker between the MM and the CM and a second linker between the CM and the TB.

32. A composition comprising the activatable target-binding protein of any one of claims 1 to 31 and a carrier.

33. The composition of claim 32, wherein the composition is a pharmaceutical composition.

34. A container, vial, syringe, injector pen, or kit comprising at least one dose of the composition of claim 32 or 33.

35. A nucleic acid comprising a sequence encoding the activatable target-binding protein of any one of claims 1 to 31.

36. A vector comprising the nucleic acid of claim 35.

37. A cell comprising the nucleic acid of claim 35 or the vector of claim 36.

38. A conjugated activatable target-binding protein comprising the activatable target-binding protein of any one of claims 1 to 31 conjugated to an agent.

39. The conjugated activatable target-binding protein of claim 38, wherein the agent is a therapeutic agent, a targeting moiety, or a detectable moiety.

40. A method of treating a subject in need thereof comprising administering to the subject a therapeutically effective amount of the activatable target-binding protein of any one of claims 1 to 31, the composition of claim 32 or 33, or the conjugated activatable target-binding protein of claim 38 or 39.

41. The method of claim 40, wherein the subject has been identified or diagnosed as having a cancer, an inflammatory condition, disorder or disease, or an autoimmune condition, disorder or disease.

42. A method of producing an activatable target-binding protein, comprising:
culturing the cell of claim 37 in a culture medium under a condition sufficient to produce the activatable target-binding protein; and
recovering the activatable target-binding protein from the cell or the culture medium.

43. The method of claim 42, further comprising isolating the activatable target-binding protein recovered from the cell or the culture medium.

44. The method of claim 43, wherein the isolating is performed using a protein purification tag and/or size exclusion chromatography.

45. The method of claim 43 or claim 44, further comprising formulating the activatable target-binding protein into a pharmaceutical composition.

46. A method of producing a dual-anchored activatable macromolecule comprising: engineering a cysteine residue at a disulfide bonding site in a masking moiety (MM) of the dual-anchored activatable macromolecule; engineering a cysteine residue at a disulfide bonding site in a target-binding protein (TB) of the dual-anchored activatable macromolecule, wherein the MM and the TB are coupled and a cleavable moiety (CM) is positioned between the MM and the TB; expressing the dual-anchored activatable macromolecule; and recovering the dual-anchored activatable macromolecule, wherein the MM and the TB are tethered at their disulfide bonding sites in the recovered dual-anchored activatable macromolecule.

47. A method of producing a dual-anchored activatable macromolecule comprising: engineering an arginine or lysine residue at an isopeptide bonding site in a masking moiety (MM) of the dual-anchored activatable macromolecule and/or engineering an aspartate or glutamate residue at an isopeptide bonding site in a target-binding protein (TB) of the dual-anchored activatable macromolecule, wherein the MM and the TB are coupled and a cleavable moiety (CM) is positioned between the MM and the TB; expressing the dual-anchored activatable macromolecule; and recovering the dual-anchored activatable macromolecule, wherein the MM and the TB are tethered at their isopeptide bonding sites in the recovered dual-anchored activatable macromolecule.

48. A method of producing a dual-anchored activatable macromolecule comprising: engineering an aspartate or glutamate residue at an isopeptide bonding site in a masking moiety (MM) of the dual-anchored activatable macromolecule and/or engineering an arginine or lysine residue at an isopeptide bonding site in a target-binding protein (TB) of the dual-anchored activatable macromolecule, wherein the MM and the TB are coupled and a cleavable moiety (CM) is positioned between the MM and the TB; expressing the dual-anchored activatable macromolecule; and recovering the dual-anchored activatable macromolecule, wherein the MM and the TB are tethered at their isopeptide bonding sites in the recovered dual-anchored activatable macromolecule.

49. A method of making a dual-anchored activatable macromolecule comprising providing a MM comprising a non-alpha-carbon covalent bond-forming amino acid configured to form a non-alpha-carbon covalent bond with a non-alpha-carbon covalent bond-forming amino acid in a TB that is coupled to the MM.

50. The method of claim 49, wherein the alpha-carbon covalent bond-forming amino acid of the MM is threonine and the alpha-carbon covalent bond-forming amino acid of the TB is glutamine.

51. The method of claim 49, wherein the alpha-carbon covalent bond-forming amino acid of the MM is glutamine and the alpha-carbon covalent bond-forming amino acid of the TB is threonine.

52. The method of claim 49, wherein the alpha-carbon covalent bond-forming amino acid of the MM is cysteine and the alpha-carbon covalent bond-forming amino acid of the TB is glutamine or tyrosine.

53. The method of claim 49, wherein the alpha-carbon covalent bond-forming amino acid of the MM is glutamine or tyrosine and the alpha-carbon covalent bond-forming amino acid of the TB is cysteine.

54. The method of claim 49, wherein the alpha-carbon covalent bond-forming amino acid of the MM is histidine and the alpha-carbon covalent bond-forming amino acid of the TB is tyrosine.

55. The method of claim 49, wherein the alpha-carbon covalent bond-forming amino acid of the MM is tyrosine and the alpha-carbon covalent bond-forming amino acid of the TB is histidine.

56. The method of claim 49, wherein the alpha-carbon covalent bond-forming amino acid of the MM is lysine and the alpha-carbon covalent bond-forming amino acid of the TB is cysteine, glutamate, or aspartate.

57. The method of claim 49, wherein the alpha-carbon covalent bond-forming amino acid of the MM is cysteine, glutamate, or aspartate and the alpha-carbon covalent bond-forming amino acid of the TB is lysine.

58. The method of claim 49, wherein the alpha-carbon covalent bond-forming amino acid of the MM is glutamine and the alpha-carbon covalent bond-forming amino acid of the TB is lysine, and the non-alpha-carbon covalent bond is an isopeptide bond between the gamma-carboxyamide group of the glutamine and the epsilon-amino group of the lysine sidechain.

59. The method of claim 49, wherein the alpha-carbon covalent bond-forming amino acid of the MM is lysine and the alpha-carbon covalent bond-forming amino acid of the TB is glutamine, and the non-alpha-carbon covalent bond is an isopeptide bond between the gamma-carboxyamide group of the glutamine and the epsilon-amino group of the lysine sidechain.

60. A method of making a dual-anchored activatable macromolecule comprising providing a MM comprising a cysteine configured to form a non-alpha-carbon covalent bond with a non-alpha-carbon covalent bond-forming amino acid in a TB that is coupled to the MM.


SEQUENCE LISTING (573 sequences)

SEQ ID NO: 1 - protein, 135 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGSHHHHHHHHHH

SEQ ID NO: 2 - protein, 178 aa, synthetic construct
QGQSGQPDRKAAVSHWQQGGGSSGGSISSGLLSGRSDNIGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGSHHHHHHHHHH

SEQ ID NO: 3 - protein, 171 aa, synthetic construct
QGQSGQPDRKAAVSHWQQGGGSSGGSPLGLAGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGSHHHHHHHHHH

SEQ ID NO: 4 - protein, 178 aa, synthetic construct
QGCSGQPDRKAAVSHWQQGGGSSGGSISSGLLSGRSDNIGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGCGTQVTVSSGGSHHHHHHHHHH

SEQ ID NO: 5 - protein, 178 aa, synthetic construct
QGCSGQPDRKAAVSHWQQGGGSSGGSISSGLLSGRSDNIGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFCGQGTQVTVSSGGSHHHHHHHHHH

SEQ ID NO: 6 - protein, 178 aa, synthetic construct
QGQSGQPCRKAAVSHWQQGGGSSGGSISSGLLSGRSDNIGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDCWGQGTQVTVSSGGSHHHHHHHHHH

SEQ ID NO: 7 - protein, 353 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 396 aa, synthetic construct
QGQSGQPDRKAAVSHWQQGGGSSGGSISSGLLSGRSDNIGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 396 aa, synthetic construct
QGCSGQPDRKAAVSHWQQGGGSSGGSISSGLLSGRSDNIGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGCGTQVTVSSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 396 aa, synthetic construct
QGCSGQPDRKAAVSHWQQGGGSSGGSISSGLLSGRSDNIGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFCGQGTQVTVSSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 396 aa, synthetic construct
QGQSGQPCRKAAVSHWQQGGGSSGGSISSGLLSGRSDNIGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDCWGQGTQVTVSSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 17 aa, synthetic construct
MRAWIFFLLCLAGRALA

SEQ ID NO: 13 - protein, 6 aa, synthetic construct
QGQSGQ

SEQ ID NO: 14 - protein, 12 aa, synthetic construct
PDRKAAVSHWQQ

SEQ ID NO: 15 - protein, 8 aa, synthetic construct
GGGSSGGS

SEQ ID NO: 16 - protein, 13 aa, synthetic construct
ISSGLLSGRSDNI

SEQ ID NO: 17 - protein, 6 aa, synthetic construct
PLGLAG

SEQ ID NO: 18 - protein, 4 aa, synthetic construct
GGGS

SEQ ID NO: 19 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSS

SEQ ID NO: 20 - skipped

SEQ ID NO: 21 - protein, 10 aa, synthetic construct
HHHHHHHHHH

SEQ ID NO: 22 - protein, 6 aa, synthetic construct
QGCSGQ

SEQ ID NO: 23 - protein, 12 aa, synthetic construct
PCRKAAVSHWQQ

SEQ ID NO: 24 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGCGTQVTVSS

SEQ ID NO: 25 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFCGQGTQVTVSS

SEQ ID NO: 26 - protein, 122 aa, synthetic construct
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDCWGQGTQVTVSS

SEQ ID NO: 27 - protein, 4 aa, synthetic construct
GGGG

SEQ ID NO: 28 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 160 aa, synthetic construct
GGSPDRKAAVSHWQQGGGGSGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGSHHHHHHHHHH

SEQ ID NO: 30 - protein, 165 aa, synthetic construct
GGSPDRKAAVSHWQQGGGGSGGGGSGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGSHHHHHHHHHH

SEQ ID NO: 31 - protein, 170 aa, synthetic construct
GGSPDRKAAVSHWQQGGGGSGGGGSGGGGSGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGSHHHHHHHHHH

SEQ ID NO: 32 - protein, 10 aa, synthetic construct
AANALAHGLF

SEQ ID NO: 33 - protein, 8 aa, synthetic construct
HMMQYARH

SEQ ID NO: 34 - protein, 4 aa, synthetic construct
AANL

SEQ ID NO: 35 - protein, 8 aa, synthetic construct
HTGRSGAL

SEQ ID NO: 36 - protein, 10 aa, synthetic construct
AANLGSGGSS

SEQ ID NO: 37 - protein, 5 aa, synthetic construct
HVPRQ

SEQ ID NO: 38 - protein, 5 aa, synthetic construct
AAPRS

SEQ ID NO: 39 - protein, 6 aa, synthetic construct
HVPRQV

SEQ ID NO: 40 - protein, 6 aa, synthetic construct
AAPRSF

SEQ ID NO: 41 - protein, 11 aa, synthetic construct
HVPRQVAPRSF

SEQ ID NO: 42 - protein, 8 aa, synthetic construct
AARGPAIH

SEQ ID NO: 43 - protein, 11 aa, synthetic construct
HVPRQVLSGRS

SEQ ID NO: 44 - protein, 8 aa, synthetic construct
AAYHLVSQ

SEQ ID NO: 45 - protein, 13 aa, synthetic construct
HVPRQVLSGRSAN

SEQ ID NO: 46 - protein, 10 aa, synthetic construct
AFPDMRSVRS

SEQ ID NO: 47 - protein, 8 aa, synthetic construct
HWHLGPPT

SEQ ID NO: 48 - protein, 7 aa, synthetic construct
AFQALRM

SEQ ID NO: 49 - protein, 8 aa, synthetic construct
IANLLSMV

SEQ ID NO: 50 - protein, 6 aa, synthetic construct
AFRHLR

SEQ ID NO: 51 - protein, 4 aa, synthetic construct
IDGR

SEQ ID NO: 52 - protein, 8 aa, synthetic construct
AGLGISST

SEQ ID NO: 53 - protein, 4 aa, synthetic construct
IEGR

SEQ ID NO: 54 - protein, 8 aa, synthetic construct
AGLGVVER

SEQ ID NO: 55 - protein, 8 aa, synthetic construct
ILNLLSMV

SEQ ID NO: 56 - protein, 4 aa, synthetic construct
AGPR

SEQ ID NO: 57 - protein, 8 aa, synthetic construct
ILPRSPAF

SEQ ID NO: 58 - protein, 4 aa, synthetic construct
AHGL

SEQ ID NO: 59 - protein, 8 aa, synthetic construct
IPFSWSRF

SEQ ID NO: 60 - protein, 5 aa, synthetic construct
AHGLF

SEQ ID NO: 61 - protein, 8 aa, synthetic construct
IQNLLSMV

SEQ ID NO: 62 - protein, 7 aa, synthetic construct
AHQALRM

SEQ ID NO: 63 - protein, 5 aa, synthetic construct
ISSGL

SEQ ID NO: 64 - protein, 10 aa, synthetic construct
AIPRVRLFDV

SEQ ID NO: 65 - protein, 6 aa, synthetic construct
ISSGLL

SEQ ID NO: 66 - protein, 5 aa, synthetic construct
ALAHG

SEQ ID NO: 67 - protein, 7 aa, synthetic construct
ISSGLLS

SEQ ID NO: 68 - protein, 6 aa, synthetic construct
ALAHGL

SEQ ID NO: 69 - protein, 13 aa, synthetic construct
ISSGLLSGRSANI

SEQ ID NO: 70 - protein, 7 aa, synthetic construct
ALAHGLF

SEQ ID NO: 71 - protein, 13 aa, synthetic construct
ISSGLLSGRSANP

SEQ ID NO: 72 - protein, 12 aa, synthetic construct
ALAHGLFAPRSF

SEQ ID NO: 73 - protein, 15 aa, synthetic construct
ISSGLLSGRSANPRG

SEQ ID NO: 74 - protein, 13 aa, synthetic construct
ALAHGLFSGRSAN

SEQ ID NO: 75 - protein, 13 aa, synthetic construct
ISSGLLSGRSDDH

SEQ ID NO: 76 - protein, 12 aa, synthetic construct
ALAHGLPTFVHL

SEQ ID NO: 77 - protein, 13 aa, synthetic construct
ISSGLLSGRSDIH

SEQ ID NO: 78 - protein, 8 aa, synthetic construct
ALGLLRLP

SEQ ID NO: 79 - protein, 13 aa, synthetic construct
ISSGLLSGRSDNH

SEQ ID NO: 80 - protein, 10 aa, synthetic construct
ALPSVKMVSE

SEQ ID NO: 81 - protein, 5 aa, synthetic construct
ALRAP

SEQ ID NO: 82 - protein, 13 aa, synthetic construct
ISSGLLSGRSDNP

SEQ ID NO: 83 - protein, 7 aa, synthetic construct
ANQALRM

SEQ ID NO: 84 - protein, 13 aa, synthetic construct
ISSGLLSGRSDQH

SEQ ID NO: 85 - protein, 8 aa, synthetic construct
ANQALRMA

SEQ ID NO: 86 - protein, 13 aa, synthetic construct
ISSGLLSGRSDTH

SEQ ID NO: 87 - protein, 10 aa, synthetic construct
APPLVKSMVV

SEQ ID NO: 88 - protein, 13 aa, synthetic construct
ISSGLLSGRSDYH

SEQ ID NO: 89 - protein, 10 aa, synthetic construct
APPSFKLVNA

SEQ ID NO: 90 - protein, 13 aa, synthetic construct
ISSGLLSGRSGNH

SEQ ID NO: 91 - protein, 4 aa, synthetic construct
APRS

SEQ ID NO: 92 - protein, 12 aa, synthetic construct
ISSGLLSGRSNI

SEQ ID NO: 93 - protein, 11 aa, synthetic construct
APRSALAHGLF

SEQ ID NO: 94 - protein, 13 aa, synthetic construct
ISSGLLSGRSNIG

SEQ ID NO: 95 - protein, 5 aa, synthetic construct
APRSF

SEQ ID NO: 96 - protein, 14 aa, synthetic construct
ISSGLLSGRSNIGS

SEQ ID NO: 97 - protein, 8 aa, synthetic construct
AQFVLTEG

SEQ ID NO: 98 - protein, 8 aa, synthetic construct
ISSGLLSS

SEQ ID NO: 99 - protein, 8 aa, synthetic construct
AQNLLGMV

SEQ ID NO: 100 - protein, 22 aa, synthetic construct
ISSGLLSSGGSGGSLSGRSDNH

SEQ ID NO: 101 - protein, 4 aa, synthetic construct
ARGP

SEQ ID NO: 102 - protein, 22 aa, synthetic construct
ISSGLLSSGGSGGSLSGRSGNH

SEQ ID NO: 103 - protein, 5 aa, synthetic construct
ARGPS

SEQ ID NO: 104 - protein, 7 aa, synthetic construct
ISSGLSS

SEQ ID NO: 105 - protein, 6 aa, synthetic construct
ARGPSF

SEQ ID NO: 106 - protein, 6 aa, synthetic construct
IVSRSA

SEQ ID NO: 107 - protein, 7 aa, synthetic construct
ARGPSFK

SEQ ID NO: 108 - protein, 12 aa, synthetic construct
KGLTGRSDRHQA

SEQ ID NO: 109 - protein, 8 aa, synthetic construct
ASGLLRFP

SEQ ID NO: 110 - protein, 10 aa, synthetic construct
KGPKVKVVTL

SEQ ID NO: 111 - protein, 10 aa, synthetic construct
ASPTMKTVGL

SEQ ID NO: 112 - protein, 12 aa, synthetic construct
KNLYGRSENNGN

SEQ ID NO: 113 - protein, 8 aa, synthetic construct
AVGLLAPP

SEQ ID NO: 114 - protein, 8 aa, synthetic construct
KRMPVQFL

SEQ ID NO: 115 - protein, 18 aa, synthetic construct
AVGLLAPPGGLSGRSANI

SEQ ID NO: 116 - protein, 8 aa, synthetic construct
LAAPLGLL

SEQ ID NO: 117 - protein, 18 aa, synthetic construct
AVGLLAPPGGLSGRSANP

SEQ ID NO: 118 - protein, 4 aa, synthetic construct
LAHG

SEQ ID NO: 119 - protein, 18 aa, synthetic construct
AVGLLAPPGGLSGRSDDH

SEQ ID NO: 120 - protein, 5 aa, synthetic construct
LAHGL

SEQ ID NO: 121 - protein, 18 aa, synthetic construct
AVGLLAPPGGLSGRSDIH

SEQ ID NO: 122 - protein, 6 aa, synthetic construct
LAHGLF

SEQ ID NO: 123 - protein, 18 aa, synthetic construct
AVGLLAPPGGLSGRSDNH

SEQ ID NO: 124 - protein, 9 aa, synthetic construct
LAPLGLQRR

SEQ ID NO: 125 - protein, 18 aa, synthetic construct
AVGLLAPPGGLSGRSDNI

SEQ ID NO: 126 - protein, 5 aa, synthetic construct
LARAG

SEQ ID NO: 127 - protein, 18 aa, synthetic construct
AVGLLAPPGGLSGRSDNP

SEQ ID NO: 128 - protein, 6 aa, synthetic construct
LARAGI

SEQ ID NO: 129 - protein, 18 aa, synthetic construct
AVGLLAPPGGLSGRSDQH

SEQ ID NO: 130 - protein, 6 aa, synthetic construct
LARAGL

SEQ ID NO: 131 - protein, 18 aa, synthetic construct
AVGLLAPPGGLSGRSDTH

SEQ ID NO: 132 - protein, 8 aa, synthetic construct
LKAAPRWA

SEQ ID NO: 133 - protein, 18 aa, synthetic construct
AVGLLAPPGGLSGRSDYH

SEQ ID NO: 134 - protein, 8 aa, synthetic construct
LKAAPRWF

SEQ ID NO: 135 - protein, 17 aa, synthetic construct
AVGLLAPPGGLSGRSNI

SEQ ID NO: 136 - protein, 8 aa, synthetic construct
LKAAPVWA

SEQ ID NO: 137 - protein, 18 aa, synthetic construct
AVGLLAPPGGLSGRSNIG

SEQ ID NO: 138 - protein, 8 aa, synthetic construct
LKAAPVWF

SEQ ID NO: 139 - protein, 19 aa, synthetic construct
AVGLLAPPGGLSGRSNIGS

SEQ ID NO: 140 - protein, 8 aa, synthetic construct
LKGRSYYY

SEQ ID NO: 141 - protein, 22 aa, synthetic construct
AVGLLAPPGGTSTSGRSANPRG

SEQ ID NO: 142 - protein, 8 aa, synthetic construct
LLAPSHRA

SEQ ID NO: 143 - protein, 17 aa, synthetic construct
AVGLLAPPSGRSANPRG

SEQ ID NO: 144 - protein, 8 aa, synthetic construct
LLEALRAL

SEQ ID NO: 145 - protein, 18 aa, synthetic construct
AVGLLAPPTSGRSANPRG

SEQ ID NO: 146 - protein, 8 aa, synthetic construct
LLESLRAL

SEQ ID NO: 147 - protein, 10 aa, synthetic construct
AVPKVRVVPE

SEQ ID NO: 148 - protein, 8 aa, synthetic construct
LLLPAHGG

SEQ ID NO: 149 - protein, 7 aa, synthetic construct
CGPPLGR

SEQ ID NO: 150 - protein, 8 aa, synthetic construct
LLLPLLGS

SEQ ID NO: 151 - protein, 7 aa, synthetic construct
CSPPLGR

SEQ ID NO: 152 - protein, 8 aa, synthetic construct
LLNALRAL

SEQ ID NO: 153 - protein, 10 aa, synthetic construct
DEVDGSGGSS

SEQ ID NO: 154 - protein, 8 aa, synthetic construct
LLNSLRAL

SEQ ID NO: 155 - skipped

SEQ ID NO: 156 - protein, 8 aa, synthetic construct
LLQALRAL

SEQ ID NO: 157 - protein, 8 aa, synthetic construct
DISHWRRS

SEQ ID NO: 158 - protein, 8 aa, synthetic construct
LLQSLRAL

SEQ ID NO: 159 - protein, 7 aa, synthetic construct
DLAHPLL

SEQ ID NO: 160 - protein, 8 aa, synthetic construct
LLSALRAL

SEQ ID NO: 161 - protein, 10 aa, synthetic construct
DLPLVKSLPS

SEQ ID NO: 162 - protein, 8 aa, synthetic construct
LLSSLRAL

SEQ ID NO: 163 - skipped

SEQ ID NO: 164 - protein, 8 aa, synthetic construct
LNGRSDNH

SEQ ID NO: 165 - protein, 12 aa, synthetic construct
DRLSGRSANHKK

SEQ ID NO: 166 - protein, 7 aa, synthetic construct
LPAGLLL

SEQ ID NO: 167 - protein, 12 aa, synthetic construct
DRLSGRSDNHKK

SEQ ID NO: 168 - protein, 8 aa, synthetic construct
LPAGLLLR

SEQ ID NO: 169 - protein, 10 aa, synthetic construct
DRPEMKSLSG

SEQ ID NO: 170 - protein, 8 aa, synthetic construct
LPAHLVLL

SEQ ID NO: 171 - protein, 10 aa, synthetic construct
DRPKVKTMDF

SEQ ID NO: 172 - protein, 8 aa, synthetic construct
LPAHLVLV

SEQ ID NO: 173 - protein, 8 aa, synthetic construct
DVAQFVLT

SEQ ID NO: 174 - protein, 8 aa, synthetic construct
LPGGLSPW

SEQ ID NO: 175 - protein, 10 aa, synthetic construct
DVPPMKTLRP

SEQ ID NO: 176 - protein, 8 aa, synthetic construct
LPSHLVLL

SEQ ID NO: 177 - protein, 8 aa, synthetic construct
DWLYWMGI

SEQ ID NO: 178 - protein, 8 aa, synthetic construct
LPSHLVLV

SEQ ID NO: 179 - protein, 8 aa, synthetic construct
DWLYWMSI

SEQ ID NO: 180 - protein, 5 aa, synthetic construct
LPTFV

SEQ ID NO: 181 - protein, 8 aa, synthetic construct
DWLYWPGI

SEQ ID NO: 182 - protein, 6 aa, synthetic construct
LPTFVH

SEQ ID NO: 183 - protein, 8 aa, synthetic construct
DWLYWPSI

SEQ ID NO: 184 - protein, 7 aa, synthetic construct
LPTFVHL

SEQ ID NO: 185 - protein, 10 aa, synthetic construct
EAPKVKALPK

SEQ ID NO: 186 - protein, 5 aa, synthetic construct
LRSGW

SEQ ID NO: 187 - protein, 10 aa, synthetic construct
EHPRVKVVSE

SEQ ID NO: 188 - protein, 4 aa, synthetic construct
LSGR

SEQ ID NO: 189 - protein, 10 aa, synthetic construct
EKPRMKLFQG

SEQ ID NO: 190 - protein, 5 aa, synthetic construct
LSGRS

SEQ ID NO: 191 - protein, 8 aa, synthetic construct
EPQALAMS

SEQ ID NO: 192 - protein, 6 aa, synthetic construct
LSGRSA

SEQ ID NO: 193 - protein, 10 aa, synthetic construct
EQPEVKMVKG

SEQ ID NO: 194 - protein, 12 aa, synthetic construct
LSGRSALAHGLF

SEQ ID NO: 195 - protein, 10 aa, synthetic construct
ERPGVKSLVL

SEQ ID NO: 196 - protein, 7 aa, synthetic construct
LSGRSAN

SEQ ID NO: 197 - protein, 8 aa, synthetic construct
ESLPVVAV

SEQ ID NO: 198 - protein, 8 aa, synthetic construct
LSGRSANI

SEQ ID NO: 199 - protein, 10 aa, synthetic construct
ESPVMKSMAL

SEQ ID NO: 200 - protein, 8 aa, synthetic construct
LSGRSANP

SEQ ID NO: 201 - protein, 5 aa, synthetic construct
ESRRW

SEQ ID NO: 202 - protein, 6 aa, synthetic construct
LSGRSD

SEQ ID NO: 203 - protein, 6 aa, synthetic construct
ESRRWM

SEQ ID NO: 204 - protein, 7 aa, synthetic construct
LSGRSDD

SEQ ID NO: 205 - protein, 7 aa, synthetic construct
ESRRWMP

SEQ ID NO: 206 - protein, 8 aa, synthetic construct
LSGRSDDH

SEQ ID NO: 207 - protein, 10 aa, synthetic construct
ETPSVKTMGR

SEQ ID NO: 208 - protein, 7 aa, synthetic construct
LSGRSDI

SEQ ID NO: 209 - protein, 12 aa, synthetic construct
ETPSVKTMGRSS

SEQ ID NO: 210 - protein, 8 aa, synthetic construct
LSGRSDIH

SEQ ID NO: 211 - protein, 10 aa, synthetic construct
FPRPLGITGL

SEQ ID NO: 212 - protein, 7 aa, synthetic construct
LSGRSDN

SEQ ID NO: 213 - protein, 8 aa, synthetic construct
FRLLDWQW

SEQ ID NO: 214 - protein, 8 aa, synthetic construct
LSGRSDNH

SEQ ID NO: 215 - protein, 8 aa, synthetic construct
GCGPPLGR

SEQ ID NO: 216 - skipped

SEQ ID NO: 217 - protein, 8 aa, synthetic construct
GCSPPLGR

SEQ ID NO: 218 - protein, 18 aa, synthetic construct
LSGRSDNHGGAVGLLAPP

SEQ ID NO: 219 - protein, 10 aa, synthetic construct
GFPHMKTFQH

SEQ ID NO: 220 - protein, 22 aa, synthetic construct
LSGRSDNHGGSGGSISSGLLSS

SEQ ID NO: 221 - protein, 12 aa, synthetic construct
GFPHMKTFQHSS

SEQ ID NO: 222 - protein, 22 aa, synthetic construct
LSGRSDNHGGSGGSQNQALRMA

SEQ ID NO: 223 - protein, 8 aa, synthetic construct
GGGPPLGR

SEQ ID NO: 224 - protein, 20 aa, synthetic construct
LSGRSDNHGGVHMPLGFLGP

SEQ ID NO: 225 - protein, 7 aa, unidentified
GGPPLGR

SEQ ID NO: 226 - protein, 8 aa, synthetic construct
LSGRSDNI

SEQ ID NO: 227 - protein, 10 aa, synthetic construct
GGQPSGMWGW

SEQ ID NO: 228 - protein, 8 aa, synthetic construct
LSGRSDNP

SEQ ID NO: 229 - protein, 7 aa, synthetic construct
GGSIDGR

SEQ ID NO: 230 - protein, 7 aa, synthetic construct
LSGRSDQ

SEQ ID NO: 231 - protein, 8 aa, synthetic construct
GGSPPLGR

SEQ ID NO: 232 - protein, 8 aa, synthetic construct
LSGRSDQH

SEQ ID NO: 233 - protein, 8 aa, synthetic construct
GGWHTGRN

SEQ ID NO: 234 - protein, 7 aa, synthetic construct
LSGRSDT

SEQ ID NO: 235 - protein, 5 aa, synthetic construct
GIAGQ

SEQ ID NO: 236 - protein, 8 aa, synthetic construct
LSGRSDTH

SEQ ID NO: 237 - protein, 10 aa, synthetic construct
GLGTPRGLFA

SEQ ID NO: 238 - protein, 7 aa, synthetic construct
LSGRSDY

SEQ ID NO: 239 - protein, 6 aa, synthetic construct
GLPTFV

SEQ ID NO: 240 - protein, 8 aa, synthetic construct
LSGRSDYH

SEQ ID NO: 241 - protein, 7 aa, synthetic construct
GLPTFVH

SEQ ID NO: 242 - protein, 8 aa, synthetic construct
LSGRSENH

SEQ ID NO: 243 - protein, 8 aa, synthetic construct
GLPTFVHL

SEQ ID NO: 244 - protein, 6 aa, synthetic construct
LSGRSG

SEQ ID NO: 245 - protein, 12 aa, synthetic construct
GLPTFVHLPRQV

SEQ ID NO: 246 - protein, 7 aa, synthetic construct
LSGRSGN

SEQ ID NO: 247 - protein, 12 aa, synthetic construct
GLSGRSDNHGSS

SEQ ID NO: 248 - protein, 8 aa, synthetic construct
LSGRSGNH

SEQ ID NO: 249 - protein, 8 aa, synthetic construct
GPEGLRVG

SEQ ID NO: 250 - protein, 22 aa, synthetic construct
LSGRSGNHGGSGGSISSGLLSS

SEQ ID NO: 251 - protein, 8 aa, synthetic construct
GPLGIAGI

SEQ ID NO: 252 - protein, 22 aa, synthetic construct
LSGRSGNHGGSGGSQNQALRMA

SEQ ID NO: 253 - protein, 12 aa, synthetic construct
GPLNGRSDNHKA

SEQ ID NO: 254 - protein, 8 aa, synthetic construct
LSGRSGNP

SEQ ID NO: 255 - protein, 12 aa, synthetic construct
GPLNGRSDNHKK

SEQ ID NO: 256 - protein, 8 aa, synthetic construct
LSGRSVTQ

SEQ ID NO: 257 - protein, 12 aa, synthetic construct
GPLNGRSDNHKR

SEQ ID NO: 258 - protein, 8 aa, synthetic construct
LSQARWRK

SEQ ID NO: 259 - protein, 12 aa, synthetic construct
GPLNGRSDNHQA

SEQ ID NO: 260 - protein, 8 aa, synthetic construct
LTFPTYIF

SEQ ID NO: 261 - protein, 12 aa, synthetic construct
GPLNGRSDNHQK

SEQ ID NO: 262 - protein, 8 aa, synthetic construct
LTFPTYWF

SEQ ID NO: 263 - protein, 12 aa, synthetic construct
GPLNGRSDNHQR

SEQ ID NO: 264 - protein, 8 aa, synthetic construct
LTGRSDRH

SEQ ID NO: 265 - protein, 12 aa, synthetic construct
GPLNGRSDNHRA

SEQ ID NO: 266 - protein, 7 aa, synthetic construct
LTGRSGA

SEQ ID NO: 267 - protein, 12 aa, synthetic construct
GPLNGRSDNHRK

SEQ ID NO: 268 - protein, 7 aa, synthetic construct
LTGRSGA

SEQ ID NO: 269 - protein, 12 aa, synthetic construct
GPLNGRSDNHRR

SEQ ID NO: 270 - protein, 8 aa, synthetic construct
LYAAPRWA

SEQ ID NO: 271 - protein, 12 aa, synthetic construct
GPLSGRSDNHKA

SEQ ID NO: 272 - protein, 8 aa, synthetic construct
LYAAPRWF

SEQ ID NO: 273 - protein, 12 aa, synthetic construct
GPLSGRSDNHKK

SEQ ID NO: 274 - protein, 8 aa, synthetic construct
LYAAPVWA

SEQ ID NO: 275 - protein, 12 aa, synthetic construct
GPLSGRSDNHKR

SEQ ID NO: 276 - protein, 8 aa, synthetic construct
LYAAPVWF

SEQ ID NO: 277 - protein, 12 aa, synthetic construct
GPLSGRSDNHQA

SEQ ID NO: 278 - protein, 8 aa, synthetic construct
LYGRSENN

SEQ ID NO: 279 - protein, 12 aa, synthetic construct
GPLSGRSDNHQK

SEQ ID NO: 280 - protein, 8 aa, synthetic construct
MDAFLESS

SEQ ID NO: 281 - protein, 12 aa, synthetic construct
GPLSGRSDNHQR

SEQ ID NO: 282 - protein, 8 aa, synthetic construct
MGLFSEAG

SEQ ID NO: 283 - protein, 12 aa, synthetic construct
GPLSGRSDNHRA

SEQ ID NO: 284 - protein, 6 aa, synthetic construct
MGPWFM

SEQ ID NO: 285 - protein, 12 aa, synthetic construct
GPLSGRSDNHRK

SEQ ID NO: 286 - protein, 8 aa, synthetic construct
MIAPVAYR

SEQ ID NO: 287 - protein, 12 aa, synthetic construct
GPLSGRSDNHRR

SEQ ID NO: 288 - protein, 6 aa, synthetic construct
MLRSGW

SEQ ID NO: 289 - protein, 6 aa, synthetic construct
GPPLGR

SEQ ID NO: 290 - protein, 7 aa, synthetic construct
MLRSGWR

SEQ ID NO: 291 - protein, 8 aa, synthetic construct
GPQGIAGQ

SEQ ID NO: 292 - protein, 8 aa, synthetic construct
MLRSGWRG

SEQ ID NO: 293 - protein, 8 aa, synthetic construct
GPQGLLGA

SEQ ID NO: 294 - protein, 8 aa, synthetic construct
MLRSGWRL

SEQ ID NO: 295 - protein, 6 aa, synthetic construct
GPRSFG

SEQ ID NO: 296 - protein, 8 aa, synthetic construct
MLRSGWRS

SEQ ID NO: 297 - protein, 7 aa, synthetic construct
GPRSFGL

SEQ ID NO: 298 - protein, 8 aa, synthetic construct
MTFPTYIF

SEQ ID NO: 299 - protein, 8 aa, synthetic construct
GPSHLVLT

SEQ ID NO: 300 - protein, 8 aa, synthetic construct
MTFPTYWF

SEQ ID NO: 301 - protein, 4 aa, synthetic construct
GPTN

SEQ ID NO: 302 - protein, 12 aa, synthetic construct
NHRIGRSDNHRR

SEQ ID NO: 303 - protein, 11 aa, synthetic construct
GPTNALAHGLF

SEQ ID NO: 304 - protein, 10 aa, synthetic construct
NMPSFKLVTG

SEQ ID NO: 305 - protein, 5 aa, synthetic construct
GRSML

SEQ ID NO: 306 - protein, 12 aa, synthetic construct
NTLSGRSENHSG

SEQ ID NO: 307 - protein, 6 aa, synthetic construct
GRSMLL

SEQ ID NO: 308 - protein, 12 aa, synthetic construct
NTLSGRSGNHGS

SEQ ID NO: 309 - protein, 7 aa, synthetic construct
GRSMLLG

SEQ ID NO: 310 - protein, 10 aa, synthetic construct
NZPRVRLVLP

SEQ ID NO: 311 - protein, 8 aa, synthetic construct
GRSMLLGG

SEQ ID NO: 312 - protein, 8 aa, synthetic construct
PAGLWLDP

SEQ ID NO: 313 - protein, 8 aa, synthetic construct
GRSMLLGP

SEQ ID NO: 314 - protein, 5 aa, synthetic construct
PAGRR

SEQ ID NO: 315 - protein, 8 aa, synthetic construct
GRSMLLGS

SEQ ID NO: 316 - protein, 6 aa, synthetic construct
PAGRRS

SEQ ID NO: 317 - protein, 7 aa, synthetic construct
GRSMLLP

SEQ ID NO: 318 - protein, 7 aa, synthetic construct
PAGRRSL

SEQ ID NO: 319 - protein, 8 aa, synthetic construct
GRSMLLPG

SEQ ID NO: 320 - protein, 8 aa, synthetic construct
PASLWYTQ

SEQ ID NO: 321 - protein, 8 aa, synthetic construct
GRSMLLPP

SEQ ID NO: 322 - protein, 8 aa, synthetic construct
PESRRWMP

SEQ ID NO: 323 - protein, 8 aa, synthetic construct
GRSMLLPS

SEQ ID NO: 324 - protein, 6 aa, synthetic construct
PFHLSR

SEQ ID NO: 325 - protein, 7 aa, synthetic construct
GRSMLLS

SEQ ID NO: 326 - protein, 6 aa, synthetic construct
PHGFFQ

SEQ ID NO: 327 - protein, 8 aa, synthetic construct
GRSMLLSG

SEQ ID NO: 328 - protein, 7 aa, synthetic construct
PLARAGI

SEQ ID NO: 329 - protein, 8 aa, synthetic construct
GRSMLLSP

SEQ ID NO: 330 - protein, 7 aa, synthetic construct
PLARAGL

SEQ ID NO: 331 - protein, 8 aa, synthetic construct
GRSMLLSS

SEQ ID NO: 332 - protein, 4 aa, synthetic construct
PLGL

SEQ ID NO: 333 - protein, 6 aa, synthetic construct
GRSMLM

SEQ ID NO: 334 - protein, 7 aa, synthetic construct
GRSMLMG

SEQ ID NO: 335 - protein, 6 aa, synthetic construct
PLGLWA

SEQ ID NO: 336 - protein, 8 aa, synthetic construct
GRSMLMGG

SEQ ID NO: 337 - protein, 7 aa, synthetic construct
PLGVRGK

SEQ ID NO: 338 - protein, 8 aa, synthetic construct
GRSMLMGP

SEQ ID NO: 339 - protein, 8 aa, synthetic construct
PLTGRSGG

SEQ ID NO: 340 - protein, 8 aa, synthetic construct
GRSMLMGS

SEQ ID NO: 341 - protein, 12 aa, synthetic construct
PLTGRSGGGGSS

SEQ ID NO: 342 - protein, 7 aa, synthetic construct
GRSMLMP

SEQ ID NO: 343 - protein, 5 aa, synthetic construct
PPLGR

SEQ ID NO: 344 - protein, 8 aa, synthetic construct
GRSMLMPG

SEQ ID NO: 345 - protein, 10 aa, synthetic construct
PPPDMKLFPG

SEQ ID NO: 346 - protein, 8 aa, synthetic construct
GRSMLMPP

SEQ ID NO: 347 - protein, 10 aa, synthetic construct
PPPEVRSFSV

SEQ ID NO: 348 - protein, 8 aa, synthetic construct
GRSMLMPS

SEQ ID NO: 349 - protein, 10 aa, synthetic construct
PPPVLKLLEW

SEQ ID NO: 350 - protein, 7 aa, synthetic construct
GRSMLMS

SEQ ID NO: 351 - protein, 12 aa, synthetic construct
PPSIARSDNLAN

SEQ ID NO: 352 - protein, 8 aa, synthetic construct
GRSMLMSG

SEQ ID NO: 353 - protein, 8 aa, synthetic construct
PQHRIVSF

SEQ ID NO: 354 - protein, 8 aa, synthetic construct
GRSMLMSP

SEQ ID NO: 355 - protein, 8 aa, synthetic construct
PRFKIIGG

SEQ ID NO: 356 - protein, 8 aa, synthetic construct
GRSMLMSS

SEQ ID NO: 357 - protein, 8 aa, synthetic construct
PRFRIIGG

SEQ ID NO: 358 - protein, 8 aa, synthetic construct
GSGPPLGR

SEQ ID NO: 359 - protein, 10 aa, synthetic construct
PRPFVKSVDQ

SEQ ID NO: 360 - protein, 7 aa, synthetic construct
GSPPLGR

SEQ ID NO: 361 - protein, 4 aa, synthetic construct
PRQV

SEQ ID NO: 362 - protein, 8 aa, synthetic construct
GSSPPLGR

SEQ ID NO: 363 - protein, 4 aa, synthetic construct
PRSF

SEQ ID NO: 364 - protein, 12 aa, synthetic construct
GTGRGPSWVGSS

SEQ ID NO: 365 - protein, 10 aa, synthetic construct
PSPPVKMMPE

SEQ ID NO: 366 - protein, 4 aa, synthetic construct
PTNL

SEQ ID NO: 367 - protein, 10 aa, synthetic construct
PTNGGSGGSS

SEQ ID NO: 368 - protein, 10 aa, synthetic construct
PTNLGSGGSS

SEQ ID NO: 369 - protein, 10 aa, synthetic construct
RVPKVKVMLD

SEQ ID NO: 370 - protein, 8 aa, synthetic construct
PVGYTSSL

SEQ ID NO: 371 - protein, 8 aa, synthetic construct
SAGFSLPA

SEQ ID NO: 372 - protein, 10 aa, synthetic construct
PVPRLKLIKD

SEQ ID NO: 373 - protein, 8 aa, synthetic construct
SAPAVESE

SEQ ID NO: 374 - protein, 8 aa, synthetic construct
PVQPIGPQ

SEQ ID NO: 375 - protein, 10 aa, synthetic construct
SAPYFRMMDM

SEQ ID NO: 376 - protein, 8 aa, synthetic construct
QALAMSAI

SEQ ID NO: 377 - protein, 8 aa, synthetic construct
SARGPSRW

SEQ ID NO: 378 - protein, 7 aa, synthetic construct
QFQALRM

SEQ ID NO: 379 - protein, 8 aa, synthetic construct
SCGPPLGR

SEQ ID NO: 380 - protein, 10 aa, synthetic construct
QGPMFKSLWD

SEQ ID NO: 381 - protein, 8 aa, synthetic construct
SCSPPLGR

SEQ ID NO: 382 - protein, 8 aa, synthetic construct
QGRAITFI

SEQ ID NO: 383 - protein, 8 aa, synthetic construct
SGGPLGVR

SEQ ID NO: 384 - protein, 7 aa, synthetic construct
QHQALRM

SEQ ID NO: 385 - protein, 8 aa, synthetic construct
SGGPPLGR

SEQ ID NO: 386 - protein, 8 aa, synthetic construct
QNQALRIA

SEQ ID NO: 387 - protein, 7 aa, synthetic construct
SGPPLGR

SEQ ID NO: 388 - protein, 7 aa, synthetic construct
QNQALRM

SEQ ID NO: 389 - protein, 4 aa, synthetic construct
SGRS

SEQ ID NO: 390 - protein, 8 aa, synthetic construct
QNQALRMA

SEQ ID NO: 391 - protein, 5 aa, synthetic construct
SGRSA

SEQ ID NO: 392 - protein, 22 aa, synthetic construct
QNQALRMAGGSGGSLSGRSDNH

SEQ ID NO: 393 - protein, 6 aa, synthetic construct
SGRSAN

SEQ ID NO: 394 - protein, 22 aa, synthetic construct
QNQALRMAGGSGGSLSGRSGNH

SEQ ID NO: 395 - protein, 7 aa, synthetic construct
SGRSANI

SEQ ID NO: 396 - protein, 6 aa, synthetic construct
QSRRVP

SEQ ID NO: 397 - protein, 7 aa, synthetic construct
SGRSANP

SEQ ID NO: 398 - protein, 7 aa, synthetic construct
QSRRVPL

SEQ ID NO: 399 - protein, 9 aa, synthetic construct
SGRSANPRG

SEQ ID NO: 400 - protein, 7 aa, synthetic construct
QSRRVPV

SEQ ID NO: 401 - protein, 5 aa, synthetic construct
SGRSD

SEQ ID NO: 402 - protein, 6 aa, synthetic construct
QTRRVP

SEQ ID NO: 403 - protein, 6 aa, synthetic construct
SGRSDD

SEQ ID NO: 404 - protein, 7 aa, synthetic construct
QTRRVPL

SEQ ID NO: 405 - protein, 7 aa, synthetic construct
SGRSDDH

SEQ ID NO: 406 - protein, 7 aa, synthetic construct
QTRRVPV

SEQ ID NO: 407 - protein, 6 aa, synthetic construct
SGRSDI

SEQ ID NO: 408 - protein, 8 aa, synthetic construct
QYIVSRSA

SEQ ID NO: 409 - protein, 7 aa, synthetic construct
SGRSDIH

SEQ ID NO: 410 - protein, 6 aa, synthetic construct
RALRAP

SEQ ID NO: 411 - protein, 6 aa, synthetic construct
SGRSDN

SEQ ID NO: 412 - protein, 10 aa, synthetic construct
REPFMKSLPW

SEQ ID NO: 413 - protein, 7 aa, synthetic construct
SGRSDNI

SEQ ID NO: 414 - protein, 6 aa, synthetic construct
RFPLKV

SEQ ID NO: 415 - protein, 7 aa, synthetic construct
SGRSDNP

SEQ ID NO: 416 - protein, 10 aa, synthetic construct
RFPSLKSFPL

SEQ ID NO: 417 - protein, 6 aa, synthetic construct
SGRSDQ

SEQ ID NO: 418 - protein, 7 aa, synthetic construct
RFPYGVW

SEQ ID NO: 419 - protein, 7 aa, synthetic construct
SGRSDQH

SEQ ID NO: 420 - protein, 8 aa, synthetic construct
RFYRNQFF

SEQ ID NO: 421 - protein, 6 aa, synthetic construct
SGRSDT

SEQ ID NO: 422 - protein, 4 aa, synthetic construct
RGPA

SEQ ID NO: 423 - protein, 7 aa, synthetic construct
SGRSDTH

SEQ ID NO: 424 - protein, 8 aa, synthetic construct
RGPAFNPM

SEQ ID NO: 425 - protein, 6 aa, synthetic construct
SGRSDY

SEQ ID NO: 426 - protein, 8 aa, synthetic construct
RGPATPIM

SEQ ID NO: 427 - protein, 7 aa, synthetic construct
SGRSDYH

SEQ ID NO: 428 - protein, 7 aa, synthetic construct
RGPKLYW

SEQ ID NO: 429 - protein, 5 aa, synthetic construct
SGRSG

SEQ ID NO: 430 - protein, 6 aa, synthetic construct
RHLAKL

SEQ ID NO: 431 - protein, 6 aa, synthetic construct
SGRSGN

SEQ ID NO: 432 - protein, 8 aa, synthetic construct
RIGRSDNH

SEQ ID NO: 433 - protein, 7 aa, synthetic construct
SGRSGNH

SEQ ID NO: 434 - protein, 8 aa, synthetic construct
RKMPNITV

SEQ ID NO: 435 - protein, 8 aa, synthetic construct
SGSPPLGR

SEQ ID NO: 436 - protein, 14 aa, synthetic construct
RKSSIIIRMRDVVL

SEQ ID NO: 437 - protein, 8 aa, synthetic construct
SIARSDNL

SEQ ID NO: 438 - protein, 8 aa, synthetic construct
RKTVQHWW

SEQ ID NO: 439 - protein, 14 aa, synthetic construct
SISSGLLSGRSDNI

SEQ ID NO: 440 - protein, 8 aa, synthetic construct
RLGRSDNN

SEQ ID NO: 441 - protein, 6 aa, synthetic construct
SNPFKY

SEQ ID NO: 442 - protein, 8 aa, synthetic construct
RMHLRSLG

SEQ ID NO: 443 - protein, 8 aa, synthetic construct
SPLPLRVP

SEQ ID NO: 444 - protein, 8 aa, synthetic construct
RPLARAGI

SEQ ID NO: 445 - protein, 8 aa, synthetic construct
SPLPLRVP

SEQ ID NO: 446 - protein, 8 aa, synthetic construct
RPLARAGL

SEQ ID NO: 447 - protein, 8 aa, synthetic construct
SPLTGRSG

SEQ ID NO: 448 - protein, 12 aa, synthetic construct
RPLNGRSDNHKA

SEQ ID NO: 449 - protein, 6 aa, synthetic construct
SPPLGR

SEQ ID NO: 450 - protein, 12 aa, synthetic construct
RPLNGRSDNHKK

SEQ ID NO: 451 - protein, 5 aa, synthetic construct
SRRVP

SEQ ID NO: 452 - protein, 12 aa, synthetic construct
RPLNGRSDNHKR

SEQ ID NO: 453 - protein, 6 aa, synthetic construct
SRRVPL

SEQ ID NO: 454 - protein, 12 aa, synthetic construct
RPLNGRSDNHQA

SEQ ID NO: 455 - protein, 6 aa, synthetic construct
SRRVPV

SEQ ID NO: 456 - protein, 12 aa, synthetic construct
RPLNGRSDNHQK

SEQ ID NO: 457 - protein, 8 aa, synthetic construct
SSGPPLGR

SEQ ID NO: 458 - protein, 12 aa, synthetic construct
RPLNGRSDNHQR

SEQ ID NO: 459 - protein, 7 aa, synthetic construct
SSPPLGR

SEQ ID NO: 460 - protein, 12 aa, synthetic construct
RPLNGRSDNHRA

SEQ ID NO: 461 - protein, 8 aa, synthetic construct
SSRGPAYL

SEQ ID NO: 462 - protein, 12 aa, synthetic construct
RPLNGRSDNHRK

SEQ ID NO: 463 - protein, 9 aa, synthetic construct
SSRHRRALD

SEQ ID NO: 464 - protein, 12 aa, synthetic construct
RPLNGRSDNHRR

SEQ ID NO: 465 - protein, 15 aa, synthetic construct
SSSFDKGKYKKGDDA

SEQ ID NO: 466 - protein, 12 aa, synthetic construct
RPLSGRSDNHKA

SEQ ID NO: 467 - protein, 15 aa, synthetic construct
SSSFDKGKYKRGDDA

SEQ ID NO: 468 - protein, 12 aa, synthetic construct
RPLSGRSDNHKK

SEQ ID NO: 469 - protein, 8 aa, synthetic construct
SSSPPLGR

SEQ ID NO: 470 - protein, 12 aa, synthetic construct
RPLSGRSDNHKR

SEQ ID NO: 471 - protein, 8 aa, synthetic construct
STFPFGMF

SEQ ID NO: 472 - protein, 12 aa, synthetic construct
RPLSGRSDNHQA

SEQ ID NO: 473 - protein, 6 aa, synthetic construct
STVFHM

SEQ ID NO: 474 - protein, 12 aa, synthetic construct
RPLSGRSDNHQK

SEQ ID NO: 475 - protein, 6 aa, synthetic construct
SVHHLI

SEQ ID NO: 476 - protein, 12 aa, synthetic construct
RPLSGRSDNHQR

SEQ ID NO: 477 - protein, 8 aa, synthetic construct
SVSGLLSH

SEQ ID NO: 478 - protein, 12 aa, synthetic construct
RPLSGRSDNHRA

SEQ ID NO: 479 - protein, 8 aa, synthetic construct
SVSGLLSS

SEQ ID NO: 480 - protein, 12 aa, synthetic construct
RPLSGRSDNHRK

SEQ ID NO: 481 - protein, 8 aa, synthetic construct
SVSGLRSH

SEQ ID NO: 482 - protein, 12 aa, synthetic construct
RPLSGRSDNHRR

SEQ ID NO: 483 - protein, 8 aa, synthetic construct
SVSGLRSS

SEQ ID NO: 484 - protein, 8 aa, synthetic construct
RPSPMWAY

SEQ ID NO: 485 - protein, 4 aa, synthetic construct
TARG

SEQ ID NO: 486 - protein, 8 aa, synthetic construct
RRHDGLRA

SEQ ID NO: 487 - protein, 5 aa, synthetic construct
TARGP

SEQ ID NO: 488 - protein, 8 aa, synthetic construct
RRHDGLRS

SEQ ID NO: 489 - protein, 12 aa, synthetic construct
TARGPALAHGLF

SEQ ID NO: 490 - protein, 8 aa, synthetic construct
RSLVFAPI

SEQ ID NO: 491 - protein, 6 aa, synthetic construct
TARGPS

SEQ ID NO: 492 - protein, 8 aa, synthetic construct
RSPSRLKC

SEQ ID NO: 493 - protein, 7 aa, synthetic construct
TARGPSF

SEQ ID NO: 494 - protein, 8 aa, synthetic construct
VAGRSMRP

SEQ ID NO: 495 - protein, 8 aa, synthetic construct
TARGPSFK

SEQ ID NO: 496 - protein, 10 aa, synthetic construct
VAPQLKSLVP

SEQ ID NO: 497 - protein, 7 aa, synthetic construct
TARGPSW

SEQ ID NO: 498 - protein, 8 aa, synthetic construct
VAQFVLTE

SEQ ID NO: 499 - protein, 7 aa, synthetic construct
TARGPSW

SEQ ID NO: 500 - protein, 10 aa, synthetic construct
VHMPLGFLGP

SEQ ID NO: 501 - protein, 10 aa, synthetic construct
TARGPVPRQV

SEQ ID NO: 502 - protein, 20 aa, synthetic construct
VHMPLGFLGPGGLSGRSDNH

SEQ ID NO: 503 - protein, 4 aa, synthetic construct
TFVH

SEQ ID NO: 504 - protein, 24 aa, synthetic construct
VHMPLGFLGPGGTSTSGRSANPRG

SEQ ID NO: 505 - protein, 12 aa, synthetic construct
TGLSGRSVTQTS

SEQ ID NO: 506 - protein, 10 aa, synthetic construct
VLPELRSVFS

SEQ ID NO: 507 - protein, 8 aa, synthetic construct
TGRGPSWV

SEQ ID NO: 508 - protein, 8 aa, synthetic construct
VLSKQMSF

SEQ ID NO: 509 - protein, 12 aa, synthetic construct
TLRLGRSDNNKN

SEQ ID NO: 510 - protein, 7 aa, synthetic construct
VPAGRRS

SEQ ID NO: 511 - protein, 8 aa, synthetic construct
TLSGLRSP

SEQ ID NO: 512 - protein, 8 aa, synthetic construct
VPAGRRSL

SEQ ID NO: 513 - protein, 8 aa, synthetic construct
TSGRSANP

SEQ ID NO: 514 - protein, 4 aa, synthetic construct
VPRQ

SEQ ID NO: 515 - protein, 8 aa, synthetic construct
TSGRSGNP

SEQ ID NO: 516 - protein, 5 aa, synthetic construct
VPRQV

SEQ ID NO: 517 - protein, 12 aa, synthetic construct
TSLSGRSANPRG

SEQ ID NO: 518 - protein, 5 aa, synthetic construct
VSRSA

SEQ ID NO: 519 - protein, 12 aa, synthetic construct
TSLSGRSGNPRG

SEQ ID NO: 520 - protein, 8 aa, synthetic construct
VVPEGRRS

SEQ ID NO: 521 - protein, 8 aa, synthetic construct
TSSGLRSP

SEQ ID NO: 522 - protein, 8 aa, synthetic construct
WATPRPMR

SEQ ID NO: 523 - protein, 12 aa, synthetic construct
TSTSGRSANPRG

SEQ ID NO: 524 - protein, 8 aa, synthetic construct
WDHPISLL

SEQ ID NO: 525 - protein, 22 aa, synthetic construct
TSTSGRSANPRGGGAVGLLAPP

SEQ ID NO: 526 - skipped

SEQ ID NO: 527 - protein, 24 aa, synthetic construct
TSTSGRSANPRGGGVHMPLGFLGP

SEQ ID NO: 528 - protein, 10 aa, synthetic construct
YDPZVKVVLA

SEQ ID NO: 529 - protein, 12 aa, synthetic construct
TSTSGRSGNPRG

SEQ ID NO: 530 - protein, 8 aa, synthetic construct
YGAGLGVV

SEQ ID NO: 531 - protein, 8 aa, synthetic construct
TVSGLRSP

SEQ ID NO: 532 - protein, 7 aa, synthetic construct
YIVSRSA

SEQ ID NO: 533 - protein, 8 aa, synthetic construct
YKKFVGSL

SEQ ID NO: 534 - protein, 10 aa, synthetic construct
YVPRVKALEM

SEQ ID NO: 535 - protein, 5 aa, synthetic construct
GGGGS

SEQ ID NO: 536 - protein, 5 aa, synthetic construct
GSGGS

SEQ ID NO: 537 - protein, 4 aa, synthetic construct
GGSG

SEQ ID NO: 538 - protein, 5 aa, synthetic construct
GGSGG

SEQ ID NO: 539 - protein, 5 aa, synthetic construct
GSGSG

SEQ ID NO: 540 - protein, 5 aa, synthetic construct
GSGGG

SEQ ID NO: 541 - protein, 5 aa, synthetic construct
GGGSG

SEQ ID NO: 542 - protein, 5 aa, synthetic construct
GSSSG

SEQ ID NO: 543 - protein, 11 aa, synthetic construct
GSSGGSGGSGG

SEQ ID NO: 544 - protein, 8 aa, synthetic construct
GGGSGGGS

SEQ ID NO: 545 - protein, 12 aa, synthetic construct
GGGSGGGSGGGS

SEQ ID NO: 546 - protein, 15 aa, synthetic construct
GGGGSGGGGSGGGGS

SEQ ID NO: 547 - protein, 25 aa, synthetic construct
GGGGSGGGGSGGGGSGGGGSGGGGS

SEQ ID NO: 548 - protein, 10 aa, synthetic construct
GGGGSGGGGS

SEQ ID NO: 549 - protein, 7 aa, synthetic construct
GGGGSGS

SEQ ID NO: 550 - protein, 17 aa, synthetic construct
GGGGSGGGGSGGGGSGS

SEQ ID NO: 551 - protein, 12 aa, synthetic construct
GGSLDPKGGGGS

SEQ ID NO: 552 - protein, 20 aa, synthetic construct
PKSCDKTHTCPPCPAPELLG

SEQ ID NO: 553 - protein, 17 aa, synthetic construct
SKYGPPCPPCPAPEFLG

SEQ ID NO: 554 - protein, 12 aa, synthetic construct
GKSSGSGSESKS

SEQ ID NO: 555 - protein, 14 aa, synthetic construct
GSTSGSGKSSEGKG

SEQ ID NO: 556 - protein, 18 aa, synthetic construct
GSTSGSGKSSEGSGSTKG

SEQ ID NO: 557 - protein, 18 aa, synthetic construct
GSTSGSGKPGSGEGSTKG

SEQ ID NO: 558 - protein, 16 aa, synthetic construct
GSTSGSGKPGSSEGST

SEQ ID NO: 559 - protein, 16 aa, synthetic construct
GSTSGSGKPGSSEGST

SEQ ID NO: 560 - protein, 11 aa, synthetic construct
GGGGSGGGGSS

SEQ ID NO: 561 - protein, 15 aa, synthetic construct
GGGSSGGSGGSSGGS

SEQ ID NO: 562 - protein, 442 aa, synthetic construct
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSAAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 563 - protein, 264 aa, synthetic construct
QGQSGSGIALCPSHFCQLPQTGGGSSGGSGGSGGISSGLLSGRSDNHGGSDIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 564 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 565 - protein, 15 aa, synthetic construct
GIALCPSHFCQLPQT

SEQ ID NO: 566 - protein, 6 aa, synthetic construct
QGQSGS

SEQ ID NO: 567 - protein, 17 aa, synthetic construct
QGQSGQPDRKAAVSHWQ

SEQ ID NO: 568 - protein, 214 aa, synthetic construct
YELTQPPSASVNVGETVKITCSGDQLPKYFADWFHQRSDQTILQVIYDDNKRPSGIPERISGSSSGTTATLTIRDVRAEDEGDYYCFSGYVDSDSKLYVFGSGTQLTVLGGPKSSPKVTVFPPSPEELRTNKATLVCLVNDFYPGSATVTWKANGATINDGVKTTKPSKQGQNYMTSSYLSLTADQWKSHNRVSCQVTHEGETVEKSLSPAECL

SEQ ID NO: 569 - protein, 218 aa, synthetic construct
EVRLLESGGGLVKPEGSLKLSCVASGFTFSDYFMSWVRQAPGKGLEWVAHIYTKSYNYATYYSGSVKGRFTISRDDSRSMVYLQMNNLRTEDTATYYCTRDGSGYPSLDFWGQGTQVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 570 - protein, 218 aa, synthetic construct
DIQLTQSPSSLSASVGDRVTITCRASESVDAYGISFMNWFQQKPGKAPKLLIYAASNQGSGVPSRFSGSGSGTDFTLTISSMQPEDFATYYCQQSKDVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 571 - protein, 217 aa, synthetic construct
EVQLVESGGGLVQPGGSLRLSCAASGFTFSGYAMSWVRQAPGKGLEWVAYISNSGGNAHYADSVKGRFTISRDNSKNTLYLQMNSLRAEDTAVYYCTREDYGTSPFVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 572 - protein, 450 aa, synthetic construct
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 573 - protein, 219 aa, synthetic construct
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC